(19) United States
(12) Patent Application Publication
(10) Pub. No.: US 2010/0129406 A1
(43) Pub. Date: May 27, 2010

(54) HOLIN-ENHANCED VACCINES AND REAGENTS, AND METHODS OF USE THEREOF

(75) Inventors: Peter M. Lauer, Albany, CA (US); Thomas W. Dubensky, JR., Piedmont, CA (US); William S. Luckett, Richmond, CA (US); William G. Hanson, Walnut Creek, CA (US)

Correspondence Address: BioTechnology Law Group;12707 High Bluff Drive, Suite 200, San Diego, CA, 92130-2037, US

(73) Assignee: ANZA THERAPEUTICS, INC., Concord, CA (US)

(21) Appl. No.: 12/439,287

(22) PCT Filed: Aug. 31, 2007
(86) PCT No.: PCT/US2007/019211

§ 371 Date: Jan. 22, 2010

Related U.S. Application Data
(60) Provisional application No. 60/841,705, filed on Sep. 1, 2006.

(51) Int. Cl. A61K 39/02; C12N 1/21

(52) U.S. Cl. 424/235.1; 435/252.3

(57) ABSTRACT

The invention provides Listeria that, in addition to comprising polynucleotides that encode heterologous polypeptides such as tumor or infectious agent antigens, have been modified to express holin proteins that facilitate the delivery of the heterologous polypeptides, or polynucleotides encoding the same, outside of the bacteria. In some particular embodiments, the Listeria generate viral-derived, self-replicating RNAs that direct expression of the heterologous polypeptides in the cytosol of infected cells. Methods of using the Listeria, and compositions thereof, to induce immune response and/or in the prevention or treatment of disease are also provided. Methods of producing the bacteria are also provided.

### CROSS-REFERENCE TO RELATED APPLICATIONS

This application claims the priority benefit of U.S. Provisional Application No. 60/841,705, filed Sep. 1, 2006, the contents of which are hereby incorporated by reference herein in their entirety.

### FIELD OF THE INVENTION

This invention relates generally to Listeria that express holin proteins and are useful for the delivery of heterologous polynucleotides and/or polypeptides. In particular, the Listeria are useful for delivery of heterologous polynucleotides and/or polypeptides to the cytosol of infected cells and in vaccines.

### BACKGROUND OF THE INVENTION

Cancers and infections can be treated by administering reagents that modulate the immune system. These reagents include vaccines, cytokines, antibodies, and small molecules, such as CpG oligodeoxynucleotides and imidazoquinolines. Vaccines, including classical vaccines (inactivated whole organisms, extracts, or antigens), dendritic cell (DC) vaccines, and nucleic acid based vaccines, are all being applied to the treatment of cancers and infections (see, e.g., Robinson and Amara (2005) Nat. Med. Suppl. 11:S25-S32; Plotkin (2005) Nat. Med. Suppl. 11:S5-S11; Pashine, et al. (2005) Nat. Med. Suppl. 11:S63-S68; Larche and Wraith (2005) Nat. Med. Suppl. 11:S69-S76).

Another reagent of use in modulating the immune system is Listeria monocytogenes (L. monocytogenes; Lm). L. monocytogenes is an intracellular bacterium. Once the Listeria enters a host cell, the life cycle of the Listeria involves escape from the phagolysosome to the cytosol. L. monocytogenes' escape from the phagolysosome is mediated by listerial proteins, such as listeriolysin (LLO), PI PLC, and PC PLC (Portnoy, et al. (2002) J. Cell Biol. 158:409-414). The use of this reagent has been reported for the treatment of cancers and tumors (see, e.g., Brockstedt, et al. (2004) Proc. Natl. Acad. Sci. USA 101:13832-13837; Brockstedt, et al (2005) Nature Med. 11:853-860); Starks, et al. (2004) J. Immunol. 173:420-427; Shen, et al. (1995) Proc. Natl. Acad. Sci. USA 92:3987-3991). Listeria-based vaccines are also reported, e.g., in U.S. Patent Publication Nos. 2005/0281783, 2005/0249748, 2004/0228877, and 2004/0197343, each of which is incorporated by reference herein in its entirety.

Improvements in the methods for Listeria-mediated delivery of heterologous antigens to the cytosol of infected cells, especially antigen-presenting cells, are desired for the development of vaccines of increased efficacy.

### SUMMARY OF THE INVENTION

The invention provides Listeria that are modified to express holin proteins and that are useful as heterologous antigen delivery vectors. In some embodiments, the delivery of heterologous polypeptides and/or polynucleotides from the Listeria to the cytosol of infected cells is enhanced or mediated by the holin proteins. Compositions such as pharmaceutical compositions and vaccines comprising the Listeria are provided. Methods of using the Listeria to induce immune responses or treat or prevent disease in mammals are further provided. Polynucleotides useful in the construction of the modified Listeria are also provided.

In one aspect, the invention provides a Listeria bacterium (e.g., Listeria monocytogenes) comprising a first polynucleotide comprising a polynucleotide encoding a holin protein. In some embodiments, the bacterium further comprises a first promoter, wherein the first promoter is operably linked to the polynucleotide encoding the holin protein. In some embodiments, the bacterium further comprises a second polynucleotide comprising a polynucleotide encoding a heterologous polypeptide (e.g., a heterologous polypeptide comprising an antigen). The second polynucleotide may further comprise a promoter that is operably linked to the polynucleotide encoding the heterologous polypeptide. In some embodiments, the bacterium may instead comprise a second polynucleotide comprising a polynucleotide encoding a self-replicating RNA which comprises a polynucleotide encoding a heterologous polypeptide. The second polynucleotide may, in some embodiments, further comprise a promoter that is operably linked to the polynucleotide encoding the self-replicating RNA. In some embodiments, the bacterium expresses the holin protein. In some embodiments, when the bacterium expresses the holin protein, the bacterium remains viable. In some embodiments, when the bacterium expresses the holin protein, the holin protein is expressed at at a level that does not substantially impair the growth of the bacterium and/or that does not lyse the cell membrane of the bacteria. In some embodiments, the holin protein is derived from a non-listerial bacterium or from a bacteriophage that is not a listeriophage. In some embodiments, the bacteria comprises: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a heterologous polypeptide, and (ii) a second promoter, wherein the second promoter is operably linked to the polynucleotide encoding the heterologous polypeptide Populations comprising the Listeria are also provided. Pharmaceutical compositions, immunogenic compositions, and vaccines comprising the Listeria are further provided. In addition, methods of using the Listeria to induce an immune response to an antigen in a mammal, to treat a disease (e.g., cancer or an infectious disease) in a mammal, or to prevent a disease (e.g., cancer or an infectious disease) in a mammal are also provided.

In another aspect, the invention provides a population of bacteria comprising a plurality of Listeria bacteria, wherein each of the Listeria bacteria comprises a first polynucleotide comprising a polynucleotide encoding a holin protein. In some embodiments, the first polnucleotide further comprises a first promoter that is operably linked to the polynucleotide encoding the holin protein. In some embodiments, each of the Listeria bacteria further comprise (b) a second polynucleotide comprising (i) a polynucleotide encoding a heterologous polypeptide. The second polynucleotide may also optionally comprise a promoter that is operably linked to the polynucleotide encoding the heterologous polypeptide. In some embodiments, the bacteria express the holin protein. In some embodiments, when the Listeria bacteria express the holin protein, expression of the holin protein does not substantially impair the net growth of the population. In some embodiments, when the Listeria bacteria express the holin protein, a substantial number of the Listeria bacteria are not lysed. In some embodiments, the Listeria bacteria comprises: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a heterologous polypeptide, and (ii) a second promoter, wherein the second promoter is operably linked to the polynucleotide encoding the heterologous polypeptide. Pharmaceutical compositions, immunogenic compositions, and vaccines comprising the Listeria are further provided. In addition, methods of using the Listeria to induce an immune response to an antigen in a mammal, to treat a disease (e.g., cancer or an infectious disease) in a mammal, or to prevent a disease (e.g., cancer or an infectious disease) in a mammal are also provided.

In some embodiments of each of the aforementioned aspects, as well as other aspects described herein, the second polynucleotide encodes an RNA transcript comprising an expression cassette derived from an ssRNA positive-strand virus, wherein the expression cassette encodes the heterologous polypeptide. In some embodiments, the second polynucleotide comprises a replicon derived from a ss RNA positive-strand virus, which has been adapted to encode the heterologous polypeptide (including, but not limited to, a tumor antigen, or infectious disease antigen). In some embodiments, the virus is from a family selected from the group consisting of Togaviridae, Flaviviridae, and Picornaviridae. In some embodiments, the virus is a togavirsu, flavivirus, pestivirus, and picornavirus. In some embodiments, the RNA transcripts are capable of cap-independent translation.

In some embodiments of each of the aforementioned aspects, as well as other aspects described herein, when the holin protein is expressed by the bacterium or bacteria, at least some of the second polynucleotide, heterologous polypeptide, RNA transcript of the second polynucleotide, and/or self-replicating RNA is released from the bacterium or bacteria.

In some embodiments of each of the aforementioned aspects, as well as other aspects described herein, the heterologous polypeptide does not comprise a signal peptide sequence.

In some embodiments of each of the aforementioned aspects, as well as other aspects described herein, the Listeria do not express a lysin protein. In some embodiments, the Listeria do not contain a polynucleotide comprising a polynucleotide (e.g., a recombinant polynucleotide) encoding a lysin protein.

In some alternative embodiments of each of the aforementioned aspects, as well as other aspects described herein, the Listeria comprise a polynucleotide comprising a polynucleotide (e.g., a recombinant polynucleotide) encoding a lysin protein. In some embodiments, the polynucleotide comprising the polynucleotide encoding the lysin protein is operably linked to a promoter.

In some embodiments of each of the aforementioned aspects, as well as other aspects described herein, the bacterium or bacteria express the holin protein when the bacterium is in the cytosol of an infected host cell.

In some embodiments, of each of the aforementioned aspects, as well as other aspects described herein, the first and/or second polynucleotide is in the genomic DNA of the Listeria bacterium. In some alternative embodiments, the first and/or second polynucleotide is on a plasmid.

In some embodiments, of each of the aforementioned aspects, as well as other aspects described herein, the first promoter is a prfA-dependent promoter (including, but not limited to, an actA promoter).

In some embodiments, of each of the aforementioned aspects, as well as other aspects described herein, the second promoter is a eukaryotic promoter. In some alternative embodiments, the promoter is a prokaryotic promoter.

In some embodiments, of each of the aforementioned aspects, as well as other aspects described herein, the holin protein is expressed in the bacterium when the bacterium or bacteria is in the cytosol of a host cell.

In some embodiments, of each of the aforementioned aspects, as well as other aspects described herein, the heterologous polypeptide comprises an antigen, such as a tumor antigen, or an antigenic fragment or variant thereof, or an infectious disease antigen, or an antigenic fragment or variant thereof.

In some embodiments, of each of the aforementioned aspects, as well as other aspects described herein, the Listeria are Listeria monocytogenes. In some embodiments, the Listeria is an attenuated form of Listeria.

Methods of producing the Listeria described herein, as well as reagents useful in in the production of the Listeria such as parental Listeria strains and polynucleotides (e.g., expression cassettes) are also provided.

Further descriptions of the aspects and embodiments described above, as well as additional embodiments and aspects of the invention are provided below.

### BRIEF DESCRIPTION OF THE FIGURES

FIG. 1A. Schematic drawing of virus-based, self-replicating, expression cassette, and holin mediated release of a nucleic acid encoding the expression cassette.

FIG. 1B. Schematic drawing of virus-based, self-replicating, expression cassette, and holin-mediated release of the expression cassette.

FIG. 1C. Schematic drawing of virus-based, self-replicating, expression cassette, and holin mediated release of the expression cassette.

FIG. 1D. Schematic drawing of holin mediated release of a macromolecule from a bacterium.

FIG. 2. Schematic diagrams of plasmids containing expression cassettes encoding holin, lysin or holing and lysin.

FIG. 3A. Bacterial growth curves.

FIG. 3B. Bacterial growth curves.

FIG. 3C. Schematic diagrams of nucleic acids.

FIG. 3D. Bacterial growth curves.

FIG. 3E. Bacterial growth curves.

FIG. 3F. Bacterial growth curves.

FIG. 4. Photographs showing fluorescent-stained bacteria and actin.

FIG. 5A. Schematic diagram of a plasmid pBHE573.

FIG. 5B. Holin mediated release of a DNA plasmid encoding luciferase out of a Listeria bacterium.

FIG. 6A. Schematic diagrams of plasmids pSH263 and pBHE530.

FIG. 6B. Holin mediated release of a DNA plasmid containing an alphavirus based, self replicating, expression vector (replicon) out of a Listeria bacterium.

FIG. 6C. Photographs of Lm-holin and Lm-holin-lysin infected mammalian cells.

FIG. 6D. Quantitation of Lm-holin and Lm-holin-lysin infected mammalian cells.

FIG. 6E. Lm ΔuvrAB-holin-lysin and KBMA Lm ΔuvrAB-holin-lysin infected mammalian cells.

FIG. 7. Mammalian cells infected with Lm containing plasmids which include an IRES.

FIG. 8A. Schematic diagram of a virus based, self-replicating, expression cassette (replicon) that contains a 5′ terminal DI 25 structure, an IRES, and an open reading frame.

FIG. 8B. RNA containing an IRES electroporated into mammalian cells.

FIG. 9. Infection of BHK cells with Lm strains expressing holin, lysin, or holin and lysin for delivering a cap-independent viral based replicon to the cytoplasm.

FIG. 10A. Infection of BHK cells with Lm strains expressing holin and lysin for delivering a cap-independent viral based replicon to the cytoplasm.

FIG. 10B. Quantitation of infection of BHK cells with Lm strains expressing holin and lysin for delivering a cap-independent viral based replicon to the cytoplasm

FIG. 11A. SIINFEKL-specific immune response.

FIG. 11B. LLO190-201-specific immune response.

FIG. 12A. Schematic diagram of plasmid pBHE558.

FIG. 12B. Holin mediated release of a polypeptide from Lm holin.

### DETAILED DESCRIPTION OF THE INVENTION

### I. General

The invention is based, in part, on the recognition that a Listeria bacterium engineered to contain a nucleic acid encoding a holin can mediate release of a nucleic acid from the bacterium. In some embodiments, the ability of Listeria to serve as a delivery vector can be improved by engineering the Listeria bacterium to contain a nucleic acid encoding a holin. In some embodiments of the invention, the holin permeabilizes the bacterial membrane, allowing release from the bacterium of antigens and nucleic acids encoding antigens. What is also encompassed in some embodiments is a Listeria bacterium containing a viral derived expression cassette, which may be released from the bacterium to the host cell's cytosol. With release, the expression cassette replicates and self amplifies, and expresses enhanced quantities of antigen.

In one aspect, the invention provides a Listeria bacterium (e.g., Listeria monocytogenes) comprising a first polynucleotide comprising a polynucleotide encoding a holin protein. In some embodiments, the bacterium further comprises a first promoter, wherein the first promoter is operably linked to the polynucleotide encoding the holin protein. In some embodiments, the bacterium further comprises a second polynucleotide comprising a polynucleotide encoding a heterologous polypeptide (e.g., a heterologous polypeptide comprising an antigen). The second polynucleotide may further comprise a promoter that is operably linked to the polynucleotide encoding the heterologous polypeptide.

In another aspect, the invention provides a population of bacteria comprising a plurality of Listeria bacteria, wherein each of the Listeria bacteria comprises a first polynucleotide comprising a polynucleotide encoding a holin protein. In some embodiments, the first polynucleotide further comprises a first promoter that is operably linked to the polynucleotide encoding the holin protein. In some embodiments, each of the Listeria bacteria further comprise (b) a second polynucleotide comprising (i) a polynucleotide encoding a heterologous polypeptide. The second polynucleotide may also optionally comprise a promoter that is operably linked to the polynucleotide encoding the heterologous polypeptide. In some embodiments, when the Listeria bacteria express the holin protein, expression of the holin protein does not substantially impair the net growth of the population. In some embodiments, when the holin protein is expressed, the holin protein is expressed at a level that does not substantially impair the net growth of the population. In some embodiments, when the Listeria bacteria express the holin protein, a substantial number of the Listeria bacteria are not lysed.

In another aspect, the invention provides a Listeria bacterium, comprising: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a heterologous polypeptide, and (ii) a second promoter, wherein the second promoter is operably linked to the polynucleotide encoding the heterologous polypeptide, wherein when the bacterium expresses the holin protein, expression of the holin protein does not substantially impair the growth of the bacterium.

In another aspect, the invention provides a Listeria bacterium, comprising: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a heterologous polypeptide, and (ii) a second promoter, wherein the second promoter is operably linked to the polynucleotide encoding the heterologous polypeptide, wherein when the Listeria bacterium expresses the holin protein, the cell membrane of the bacterium is not lysed.

In still another aspect, the invention provides a Listeria bacterium, comprising: (a) a first polynucleotide comprising (i) a polynucleotide encoding a holin protein, and (ii) a first promoter, wherein the first promoter is operably linked to the polynucleotide encoding the holin protein, and wherein the holin protein is derived from a non-listerial bacterium or from a bacteriophage that is not a listeriophage; and (b) a second polynucleotide comprising (i) a polynucleotide encoding a heterologous polypeptide, and (ii) a second promoter, wherein the second promoter is operably linked to the polynucleotide encoding the heterologous polypeptide.

In some embodiments of each of the aforementioned aspects, as well as other aspects described herein, the bacterium or bacteria further comprise RNA transcripts generated from the second polynucleotide, wherein the RNA transcripts encode the heterologous polypeptide, and wherein, when the holin protein is expressed by the bacterium, at least some of the RNA transcripts are released from the bacterium, wherein the release is holin-dependent. In some embodiments, the RNA transcripts comprise an expression cassette derived from an ssRNA positive-strand virus, wherein the expression cassette encodes the heterologous polypeptide.

In a further aspect, the invention provides a population of bacteria comprising a plurality of Listeria bacteria, wherein each of the Listeria bacteria comprises: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a heterologous polypeptide, and (ii) a second promoter, wherein the second promoter is operably linked to the polynucleotide encoding the heterologous polypeptide.

In a still further aspect, the invention provides a population of bacteria comprising a plurality of Listeria bacteria, wherein each of the Listeria bacteria comprises: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a heterologous polypeptide, and (ii) a second promoter, wherein the second promoter is operably linked to the polynucleotide encoding the heterologous polypeptide, when the

Listeria bacteria express the holin protein, expression of the holin protein does not substantially impair the net growth of the population. In some embodiments, when the Listeria bacteria express the holin protein, a substantial number of the Listeria bacteria are not lysed.

In another aspect, the invention provides a population of bacteria comprising a plurality of Listeria bacteria, wherein each of the Listeria bacteria comprises: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a heterologous polypeptide, and (ii) a second promoter, wherein the second promoter is operably linked to the polynucleotide encoding the heterologous polypeptide, and wherein, when the Listeria bacteria express the holin protein, a substantial number of the Listeria bacteria are not lysed. In some embodiments, when the Listeria bacteria express the holin protein, expression of the holin protein does not substantially impair the net growth of the population.

In a still further aspect, the invention provides a Listeria bacterium comprising: (a) a first polynucleotide comprising (i) a polynucleotide encoding a holin protein, and (ii) a first promoter, wherein the first promoter is operably linked to the polynucleotide encoding the holin protein; and (b) a second polynucleotide comprising (i) a polynucleotide encoding a self-replicating RNA, wherein the self-replicating RNA comprises a polynucleotide encoding a heterologous polypeptide, and (ii) a second promoter, wherein the second promoter is operably linked to the polynucleotide encoding the self-replicating RNA.

In some embodiments of each of the aforementioned aspects, as well as other aspects described herein, the polynucleotide(s) encoding the holin protein are recombinant polynucleotide(s).

In some embodiments of each of the aforementioned aspects, as well as other aspects described herein, the Listeria bacteria express the holin protein. In some embodiments, expression of the holin protein occurs when the bacteria are in a host cell that they have infected (e.g., in the cytosol of an infected host cell). In some embodiments, the expression of the holin protein occurs only when the Listeria are in the cytosol of an infected host cell (e.g., a mammalian cell).

In some embodiments of each of the aforementioned aspects, as well as other aspects described herein, when the holin protein is expressed by the Listeria, the second polynucleotide, an RNA transcript generated from the second polynucleotide (e.g., an mRNA, self-replicating RNA, or expression cassette derived from an ssRNA positive-strand virus), and/or the heterologous polypeptide encoded by the second polynucleotide is released from the Listeria in a holin-dependent manner. For instance, in some embodiments, when the Listeria further comprise the heterologous polypeptide and the holin protein is expressed by the Listeria, at least some of the heterologous polypeptide is released from the Listeria, wherein the release is holin-dependent. In some embodiments, the released heterologous polypeptide does not comprise a signal peptide sequence. In some embodiments, when the holin protein is expressed by the Listeria, the second polynucleotide is released from the Listeria, wherein the release is holin-dependent. In some embodiments, when the Listeria further comprises a self-replicating RNA and the holin protein is expressed by the Listeria, at least some of the self-replicating RNA is released from the Listeria, wherein the release is holin-dependent.

In some embodiments of each of the aforementioned aspects, as well as other aspects described herein, the Listeria further comprise a third polynucleotide comprising a polynucleotide (e.g. a recombinant polynucleotide) encoding a lysin protein. In some embodiments, the third polynucleotide further comprises a promoter operably linked to the polynucleotide encoding the lysin protein. In some alternative embodiments, the Listeria do not express a Lysin protein and/or comprise a polynucleotide (e.g. a recombinant polynucleotide) encoding a Lysin protein.

In some embodiments of each of the aforementioned aspects, as well as other aspects described herein, the first, second and/or third polynucleotide (if present) resides in the genomic DNA of the Listeria. Alternatively, the first, second, and/or third polynucleotide (if present) resides on a plasmid. In some embodiments, the first, second, and/or third polynucleotides are parts of the same polynucleotide molecules. In some embodiments, the first, second, and/or third polynucleotides are contained on separate polynucleotide molecules.

In some embodiments of each of the aforementioned aspects, as well as other aspects described herein, the promoter that is operably linked to the polynucleotide encoding the heterologous polypeptide is a eukaryotic promoter. Alternatively, the promoter that is operably linked to the polynucleotide encoding the heterologous polypeptide is a prokaryotic promoter.

In some embodiments of each of the aforementioned aspects, as well as other aspects described herein, the promoter that is operably linked to the polynucleotide encoding the heterologous polypeptide, the holin protein, and/or the lysin protein is a prfA-dependent promoter (e.g., an actA promoter).

In some embodiments, of each of the aforementioned aspects, as well as other aspects described herein, the Listeria is Listeria monocytogenes. In some embodiments, the Listeria is attenuated for cell-to-cell spread and/or entry into nonphagocytic cells. In some embodiments, the Listeria comprises an inactivating mutation in actA and/or inlB. In some embodiments, the Listeria is an actAinlB double deletion mutant. In some embodiments, the Listeria comprises an inactivating mutation in at least one nucleic acid repair gene, such as uvrA, uvrB, uvrC, or a recombinational repair gene. For instance, the Listeria may be a uvrAB deletion mutant. In some embodiments, the bacterium further comprises a nucleic acid cross-linking agent (e.g., a psoralen).

In some embodiments, of each of the aforementioned aspects, as well as other aspects described herein, the heterologous polypeptide comprises an antigen. The antigen may be a tumor antigen, or an antigenic fragment or variant thereof. Alternatively, the antigen may be an antigen from an infectious agent, or an antigenic fragment or variant of such an antigen.

In some embodiments, of each of the aforementioned aspects, as well as other aspects described herein, the RNA transcripts of the polynucleotide encoding the heterologous protein and/or the self-replicating RNAs comprise an expression cassette derived from an ssRNA positive-strand virus, wherein the expression cassette encodes the heterologous polypeptide. In some embodiments, the expression cassette that is derived from an ssRNA positive-strand virus comprises a replicon derived from the virus. In some embodiments, the replicon derived from the ssRNA positive-strand virus comprises sufficient genetic elements from the genome of that virus to allow for self-amplification or self-replication of the expression cassette encoding the heterologous polypeptide in a eukaryotic cell, such as a mammalian cell. In some embodiments, the virus is a virus from a family selected from the group consisting of Togaviridae, Flaviviridae, and Picornaviridae. In some embodiments, the virus is a virus selected from the group consisting of togavirus (e.g., an alphavirus), flavivirus (e.g., Kunjin virus or yellow fever virus), pestivirus (e.g., Bovine Viral Diarrhea Virus), and picornavirus (e.g., Encephalomyocarditis (EMCV) virus, poliovirus, or coxsackie virus). In some embodiments, the self-replicating RNA comprises an alphavirus replicon that expresses the heterologous polypeptide. In some embodiments, the alphavirus replicon is derived from Sindbis virus, Venezuelan Equine Encephalitis (VEE) virus, or Semliki Forest virus (SFV). In some embodiments, the self-replicating RNA comprises a picornavirus replicon that expresses the heterologous polypeptide. In some embodiments, the picornavirus replicon is derived from poliovirus. In some embodiments, the self-replicating RNA comprises a flavivirus replicon that expresses the heterologous polypeptide. In some embodiments, the self-replicating RNA is derived from Bovine Viral Diarrheal Virus (BVDV). In some embodiments, the RNA transcripts and/or self-replicating RNAs are capable of cap-independent translation (e.g., contain an IRES).

In some embodiments, the Listeria express holin and lysin and the polynucleotide encoding the heterologous polypeptide comprises a replicon derived from a poliovirus. In some embodiments, the Listeria express holin, but not lysin, and the polynucleotide encoding the heterologous polypeptide comprises a replicon derived from a sindbis virus.

In some embodiments of each of the aforementioned aspects, as well as other aspects described herein, the Listeria bacterium or bacteria further expresses a viral protein that suppresses the type I interferon (IFN) response in infected host cells upon phagosomal escape into the host cell cytoplasm. Non-limiting examples of such viral proteins are provided, e.g., in Table 11 and Example 6, below.

In some embodiments of each of the aforementioned aspects, as well as other aspects described herein, greater than 10%, greater than 25%, greater than 50%, greater than 75%, or greater than 99% of the expressed antigen is released from the from the Listeria into the cytoplasm of a eukaryotic cell upon escape of the Listeria from the phagolysosome.

In some embodiments of each of the aforementioned aspects, as well as other aspects described herein, release of the antigen from the Listeria into the cytoplasm of a eukaryotic cell upon escape of the Listeria from the phagolysosome is greater than 10% dependent on the expressed holin, greater than 25%, greater than 50%, greater than 75%, or greater than 99% dependent on the expressed holin.

Pharmaceutical compositions, immunogenic compositions, and/or vaccines comprising the Listeria of the aforementioned aspects and embodiments are further provided. In addition, methods of using the Listeria to induce an immune response to an antigen in a mammal or to treat or prevent a disease (e.g., cancer or a non-listerial infectious disease) in a mammal are also provided. In some embodiments, the pharmaceutical compositions further comprise a pharmaceutically acceptable carrier. In some embodiments, the Listeria further comprise an adjuvant.

The invention further provides bacterial populations comprising the Listeria described herein. In some embodiments, the Listeria described herein make up at least about about 10%, at least about 25%, at least about 50%, at least about 75%, at least about 90%, at least about 95%, or at least about 98% of the Listeria bacteria in a given population. In some embodiments, the Listeria described herein make up at least about about 10%, at least about 25%, at least about 50%, at least about 75%, at least about 90%, at least about 95%, or at least about 98% of the total bacteria in a given population.

In some embodiments, expression of the holin protein does not substantially impair the growth of a bacterium or the net growth of a population of bacteria. The growth of a bacterium or the net growth of a population of bacteria is said to not be substantially impaired if the growth rate is not decreased by more than about 2-fold relative to an appropriate reference or control (e.g., a parental strain). In some embodiments, the rate of growth is not decreased by more than about 10%, more than about 20%, more than about 30%, or more than about 40% (relative to an appropriate control). For instance, expression of a holin protein does not substantially impair the growth of a bacterium that expresses the holin protein (or the net growth of a population of bacteria expressing the holin protein) if the growth of the bacterium (or the net growth of the population of bacteria) is not decreased by more than about 2-fold relative to a control such as a bacterium (or bacterial population) that is not expressing the holin protein, but is otherwise generally equivalent. Generally, the growth of the bacteria are compared under identical environmental conditions, although in some instances growth following induction of expression of holin may be compared to growth prior to induction of expression of holin. In some embodiments, the control is a bacterium (or bacterial population) that does not comprise the polynucleotide encoding the holin protein. In some embodiments, the growth or net growth that is measured and compared is intracellular growth, i.e., growth in cells, such as mammalian cells (e.g., J774 cells). In some embodiments, the growth or net growth in the cytoplasm of mammalian cells is measured. Intracellular growth of a Listeria bacterium or population can be measured by light microscopy, fluorescent microscopy, colony forming unit (CFU) assays, or the quantity of listerial antigens or Listeria-specific sequences. In some embodiments, the growth or net growth is growth in broth culture or on agar. Growth in broth culture can, e.g., be measured by OD₆₀₀.

The identification of Listeria as lysed can, e.g., be made microscopically. The number of Listeria in a population that are lysed can likewise be determined microscopically. Alternatively, the level of lysis occurring within a bacterial population can instead be measured indirectly by measuring the net growth rate of the population. A substantial number of Listeria in a population are said to not be lysed when at least about 50%, at least about 60%, at least about 70%, at least about 80%, at least about 90%, or at least about 95% of the cells within an observed population are not lysed.

In some embodiments, the Listeria may comprise multiple copies of the polynucleotides encoding the holin protein. For instance, the Listeria may comprise two polynucleotides encoding the holin protein.

The term “polynucleotide” is used interchangeably herein with “nucleic acid.”

### II. Holin and Lysin Proteins and Encoding Polynucleotides

In some embodiments, the Listeria described herein comprise a polynucleotide encoding a holin protein and/or express a holin protein. In some embodiments, the polynucleotide encoding the holin protein is recombinant. In some embodiments, the Listeria comprise a polynucleotide encoding a lysin protein and/or express a lysin protein. In some embodiments, the Listeria do not comprise a polynucleotide encoding a lysin protein and/or do not express a lysin protein. In certain embodiments, the polynucleotide encoding the lysin protein is recombinant. In some embodiments, the polynucleotides encoding the holin and/or lysin are operably linked with a promoter specifically activated when the Listeria is inside a host mammalian cell (e.g., actA promoter).

The terms “holin proteins” and “holins” (used interchangeably herein) encompass membrane proteins that are capable of permeabilizing the cytoplasmic membrane of bacteria. Holins may also facilitate the activity of lysins against the peptidoglycan. The terms “lysin proteins” and “lysins” (used interchangeably herein) encompass enzymes that degrade the cell wall peptidoglycan. Lysins may have activity against the glycosidic, amide, or peptide bonds of the cell wall. Holin proteins and lysin proteins need not necessarily be full-length proteins. Thus, the holin proteins or lysin proteins encompass fragments or variants of naturally occurring holin proteins or lysin proteins, respectively, so long as the fragments or variants are functionally active. In some embodiments, holins and/or lysins are phage-encoded. In some embodiments, the sequences encoding the holins and/or lysins may have been identified in bacterial genomes.

In some embodiments, expression of a holin by Listeria results in permeabilization of the Listerial membrane, where the permeabilized membrane can allow the release of non-secretory proteins, secretory proteins (that have not yet been secreted), large and small polypeptides, nucleic acids encoding heterologous antigens, virus-derived expression cassettes, and the like.

By way of non-limiting examples, the Listeria of the invention can contain a nucleic acid encoding one holin, for example, a recombinant holin, a holin operably linked with a promoter specifically activated inside a host mammalian cell, or a holin operably linked with a prfA-dependent promoter. Moreover, the Listeria of the invention can contain a nucleic acid encoding a single transcription unit that encodes two copies of the same holin. Also, the Listeria can contain a nucleic acid encoding a single transcription unit that encodes copies of two different holin, for example, from two different types of listeriophages. To give another example, the Listeria can contain two nucleic acids that encode two different transcriptional units, where each transcriptional unit encodes a holin, and where the two holins can be the same or different. The invention contemplates Listeria encoding one, two, three, or more holins. In one aspect, a nucleic acid encoding a holin is integrated into a gene encoding a virulence factor, where the nucleic acid is operably linked to the promoter(s) of the virulence factor gene. In another aspect, a nucleic acid encoding a lysin is integrated into a gene encoding a virulence factor, where the nucleic acid is operably linked to the promoter(s) of the virulence factor gene.

A number of holins and lysins have been expressed in bacteria, as demonstrated by studies of substrate specificity of the holin (the substrate being the lipid membrane of a specific bacterium), and substrate specificity of the lysin (the substrate being peptidoglycan of a specific bacterium). These studies have demonstrated that membrane permeabilization can be accomplished by a variety of holins, and is not limited to a holin expressed by a bacteriophage that happens to specifically infect that bacterium. Also, the studies have demonstrated that bacterial cell lysis can be accomplished by the combination of a holin from a first type of bacteriophage and a lysin from an unrelated, second type of bacteriophage. Nucleic acids encoding the holin and lysin can have an origin different from the bacterium used for the lysis study. For example, bacterial lysis can occur where the holin originates from a first type of phage, for example, Lactobacillus gasseri phage phi-adh, and the lysin originates from a second type of phage, for example, Bacillus subtilis phage phi-29 (see, e.g., Henrich, et al. (1995) J. Bacteriol. 177:723-732). Bacterial lysis can occur where the holin and lysin originate from a phages that do not or cannot infect the bacterium (see, e.g., Henrich, et al. (1995) J. Bacteriol. 177:723-732; Grundling, et al. (2000) J. Bacteriol. 182:6075-6081; Steiner, et al. (1993) J. Bacteriol. 175:1038-1042; Berkmen, et al. (1997) J. Bacteriol. 179:6522-6524; Loessner, et al. (1999) J. Bacteriol. 181:4452-4460).

Control of the rate of lysis, for example, delayed lysis, can be accomplished by a number of methods. The rate of lysis can be altered by a promoter specifically activated inside a mammalian host cell that is operably linked with a nucleic acid encoding a holin. Alternatively, altered rates of bacterial cell lysis can be accomplished by expressing a nucleic acid encoding a holin that harbors a specific mutation, by expressing selected levels of a holin inhibitor (see, e.g., Grundling, et al. (2000) J. Bacteriol. 182:6082-6090), or by using a nucleic acid encoding a slower acting holin, such as phi 29 protein 14 (holin), or a faster acting holin, such as phi adh holin (see, e.g., Henrich, et al. (1995) J. Bacteriol. 177:723-723).

In some embodiments, a holin protein encoded by a polynucleotide (e.g., a recombinant polynucleotide) in the Listeria comprises, or is derived from, one of the following holins:

1-Listeria innocua Clip11262: Lin1702
>gi|16800770|ref|NP_471038.11 hypothetical protein
lin1702 [Listeria innocua Clip11262]

(SEQ ID NO: 1)

MKINWKVRMKSKVFWVSVIPLVLVLAQQLLGWFGVTIPADTVNKQALDFV
NSVFLLLGVLGVVNDPTTEGTADSELVLNRNRKDEE;
2-Lactococcus lactis subsp. cremoris SK11:
Llacc01000415
>gi|62464385|ref|ZP_00383678.1|COG5546: Small
integral membrane protein [Lactococcus lactis
subsp. cremoris SK11]

(SEQ ID NO: 2)

MNQINWKLRLKSKAFWLALLPALFLLIQAIGAPFGYKWDFVILNQQLAAV
VNAAFALLAIVGVVSDPTTSGLGDSDRVLNKDKSEENK;
3-Enterococcus faecalis V583: EF1993
>gi|29376514|ref|NP_815668.1|holin [Enterococcus
faecalis V583]

(SEQ ID NO: 3)

MINWKSRIKNKQFWLSIIPAVLLLIQVVAVPFGYKFQIEMINKQLLDVVN
ALFVVLTILGIVTDPTTPGLSDRKGDK;
4-Staphylococcus aureus subsp. aureus Mu50:
SAV1946
>gi|15924936|ref|NP_372470.1|holin homolog
[Staphylococcus aureus subsp. aureus Mu50]; phage
phi LC3

(SEQ ID NO: 4)

MINWKIRMKQKSFWVAILSAIFLFAQNIAKAIGYDIQVYTEQLTDGLNAI
LGFLVLTGVIQDPTTKGIGDSHQALEYEEPRRKY;
5-Bacillus licheniformis ATCC 14580: BL01378
>gi|52082617|ref|YP_081408.11 hypothetical protein
BL01378 [Bacillus licheniformis ATCC 14580]

(SEQ ID NO: 5)

METVLIFASVLSPIILALVELVKKTVKMPKNLIPLVSLLIGLLIGAAAYP
FTDLELVLRLWSGGLAGLTATGLFEIGKNRNARKKKNP;
6-Bacillus anthracis Sterne: BAS3785
>BAS3785

(SEQ ID NO: 6)

MDRIDVLLKAFIATFGGFCGYFLGGWDATLKILVTMAVIDYLTGMIAAGY
NGELKSKVGFKGIAKKVVLFLLVGAAAQLDSALGSNSAIREATIFFFMGN
ELLSLLENAGRMGIPLPQALTNAVEILGGKQKQEEKKGDVE;
7-Clostridium perfringens 13: CPE0383
>CPE0383

(SEQ ID NO: 7)

MEGIIICIKLGVVFLGTLFTWIFGAWDMPIVTLLVFIFLDYLTGVIKGCK
SKELCSNIGLRGITKKGLILVVLLVAVMLDRLLDNGAWMFRTLIAYFYIM
NEGISILENCAALGVPIPEFLRQALKQLNNKNNIK;
8-Corynebacterium diphtheriae NCTC13129: DIP2153

(SEQ ID NO: 8)

MPVKPASPRSHPGCPELTHERYCDAQAKAEDARYRKYQRDPKINRRYGSR
WRKIRAAYVAAHPLCEDCLEAGRYTPVQEVHHVLPIEHGGTHNFDNLQSL
CKPCHSRQSALDDDRWRQQPRVYTY;
9-Lactobacillus johnsonii NCC 533: NT01LJ1229
>LJ_1419

(SEQ ID NO: 9)

MSVNQLLDLSIVVVSVAAVVVASVYAKHKIAIDKKAAQGDLLAKAEKIVA
QSVSPLVYQAEKRGGDGEDKLTFVVQGLFLLLDMAHLPHPTMSFVKGMVE
KSVTAMKQAQSIADTVDKPKPTVVGELREVKK;
and/or
10-Streptococcus agalactiae 2603V/R: SAG1838
>SAG1838

(SEQ ID NO: 10)

MTQITDIIISSAMGILTILAGIAVQAVKVYLIKKGGEKAVLITEILAKNA
VNAVEQVATETGFKGADKLTSAKAQILAELQKYNIHMSDDDLTLFVESAV
KQMHDAWKE.

Without implying any limitation, the invention provides a Listeria containing a first nucleic acid encoding holin, a second nucleic acid encoding a lysin, or both the first and second nucleic acids, where the nucleic acids are from one or more listeriophages. The listeriophages can be, for example, A118, PSA, A511, B054, B024, B055, C707, B053, B051, D441, B604, A020, B025, B545, B653, A500, A006, B101, B056, B012, B110, B035, A502, 9425, 1313, 197, 12682, 6223, 5775, 10, 43, 43, 21, 19, 387, 1967, 2685, 4477, 575, 1652, 12029, 52, 340, 312, 108, 107, 47, 2671, 1444, 2425, 3551, 3552, 1317, 2389, 3274, 9495, 1313, 197, A511, 6223, 12682, 5775, and the like. Also provided are nucleic acids encoding a holin, lysin, or both holin and lysin, from listeriophage A511, A118, A502, A006, B653, B054 (4286), B051 (4295), B025, D441, B545, B053 (4277), B056 (5337), B101, B110, C707, B024, B012, B035, A020, A500, 4211, 2671, and 2389 (see, e.g., Zink and Loessner (1992) Appl. Environ. Microbiol. 58:296-302; Mee-Marquet, et al. (1997) Appl. Environ. Microbiol. 63:3374-3377; Loessner (1991) Appl. Environ. Microbiol. 57:882-884; Zink, et al. (1995) Microbiology 141:2577-2584; Loessner, et al. (1994) J. Gen. Virol. 75:701-710; Loessner, et al. (1994) Intervirol. 37:31-35; Ackermann, et al. (1981) Ann. Virol. (Inst. Pasteur) 132E:371-382; Chiron, et al. (1977) C.R. Soc. Biol. (Paris) 171:488-491; Ortel and Ackermann (1985) Zentralbl. Bakteriol. Hyg. Abt. 1 Orig. Reihe A 260:423-427; Rocourt, et al. (1983) Ann. Virol. (Inst. Pasteur) 134E:245-250; Rocourt, et al. (1985) Zentralbl. Bakteriol. Hyg. Abt. 1 Orig. Reihe A 259:341-350). Moreover, what is provided is EJ-1 phage holin or lysin (Haro, et al. (2003) J. Biol. Chem. 278:3929-3936). 10091] The holin is able to mediate transfer of a reagent or substance from inside the Listeria bacterium, through the lipid membrane and cell wall, to the exterior of the Listeria bacterium. The transfer-mediating function of the holin can be measured, for example, by release of a plasmid encoding luciferase or another marker, a replicon, an antigen, or a fluorescent marker. Methods for assessing permeability and pore diameters are available (see, e.g., Dijkstra and Keck (1996) J. Bacteriol. 178:5555-5562; Pink, et al. (2000) J. Bacteriol. 182:5925-5930; Sara and Sleytr (1987) J. Bacteriol. 169:4092-4098; Demchick and Koch (1996) J. Bacteriol. 178:768-773).

The invention encompasses non-listeriophage holins, including Serratia marcescens NucE (Berkmen, et al. (1997) 179:6522-6524); Staphylococcus aureus bacteriophage 187 holin (Loessner, et al. (1999) J. Bacteriol. 181:4452-4460; phage lambda holins and Lactobacillus gasseri phi-adh holin (Henrich, et al. (1995) J. Bacteriol. 177:723-732; phage phi-29 holin (Steiner, et al. (1993) J. Bacteriol. 175:1038-1042); Bacillus phage PZA holin (Loessner, et al. (1997) J. Bacteriol. 179:2845-2851); phage T4 gpt holin (Dressman and Drake (1999) J. Bacteriol. 181:4391-4396); phage PRD1 holin (Ziedaite, et al. (2005) J. Bacteriol. 187:5397-5405); Borrelia burgdorferi prophage BlyA (Damman, et al. (2000) J. Bacteriol. 182:6791-6797); Bacillus subtilis ywcE holin (Real, et al. (2005) J. Bacteriol. 187:6443-6453); Staphylococcus aureus lrgA holin and cidA holin (Brunskill and Bayles (1996) J. Bacteriol. 178:5810-5812; Rice, et al. (2004) J. Bacteriol. 186:3029-3037); Streptococcus pneumoniae cph1 holin, pneumococcal phage EJ-1 holin, phi-LC3 holin and Tuc2009 holin of Lactococcus lactis phage (Martin, et al. (1998) J. Bacteriol. 180:210-217); bacteriophage P2 gene Y holin (Ziermann, et al. (1994) J. Bacteriol. 176:4974-4984); and bacteriophage PRD1 holin P35 (Rydman and Bamford (2003) J. Bacteriol. 185:3795-3803).

Nucleic acids in bacterial genomes encoding proteins identified as holins are available (Table 1). Some of these holins can be characterized as bacterial holins, that is, holins that are not holins of cryptic phages. A cryptic phage is a phage genome integrated in the bacterial genome. These holin genes include the CidA gene of S. aureus (see, e.g., Rice, et al. (2003) J. Bacteriol. 185:2635-2643; Rice and Bayles (2003) Mol. Microbiol. 50:729-738; Bayles (2000) Trends Microbiol. 8:274-278; GenBank Acc. No. AY581892).

TABLE 1
Bacterial genomic nucleic acids encoding holins.

| Bacterium | GenBank Acc. No. |
|---|---|
| Bacillus subtilis | Z99117, nt 51006-51428. |
| Bacillus subtilis | NC_000964, nt 2263876-2264088; |
| | 3932232-3932618. |
| Bacillus anthracis strain Sterne | NC_005945, five holins, e.g., |
| | 3432919-3433284. |
| Pseudomonas entomophila | NC_008027, compl. nt |
| | 4463886-4464239. |
| Escherichia coli | BA000007, ten holins, e.g., |
| | nt 901806-902021. |
| Listeria monocytogenes strain | NC_002973 nt 142006-142428. |
| 4b F2365 | |
| Listeria innocua | AL596169, nt compl. 165378-165638. |
| Staphylococcus epidermidis | CP000029, nt 2047024-2047482. |
| Erwinia carotovora | NC_004547, compl. nt |
| | 2950159-2950467. |
| Corynebacterium diphtheriae | NC_002935, six holins, e.g., |
| | nt 3637616-3637981. |
| Corynebacterium diphtheriae | BX248360, compl. nt 129273-129650. |
| Staphylococcus aureus | AJ938182, four holins, e.g., compl. |
| | 1846356-1846793. |
| Salmonella typhimurium | AE008823 nt 16310-16529. |
| Rhodopseudomonas palustris | BX572594, nt 258068-258451. |

Lysin-encoding nucleic acids are available. Lysins from listeriophage A118 (ply118 lysin), listeriophage A500 (ply500 lysin), and listeriophage 2438 (Cp12438 lysin), from the L. monocytogenes EGDe genome, and lysins designated as L-alanyl-D-glutamate peptidases, are available (Loessner, et al. (2002) Mol. Microbiol. 44:335-349; Glaser, et al. Science 294:849-8521; Zink, et al. (1995) Microbiology 141:2577-2584; Loessner, et al. (1995) Mol. Microbiol. 16:1231-1241). The contemplated lysins encompass catalytically active lysins that are mutated, e.g., occurring as fusion proteins, truncated proteins, amino acid substituted, and amino acid deleted proteins, and the like.

What is also contemplated is a Listeria containing a nucleic acid encoding a lysin, where the lysin is, or is derived from, e.g., listeriophage A500 (GenBank Acc. No. X85009); Listeria innocua clip11262 (GenBank Acc. Nos. AL596169; AL596172, AL596163); Listeria innocua (GenBank Acc. No. X89234); Bacillus licheniformis ATCC 14580 (GenBank Acc. Nos. CP000002; AE017333); listeriophage PSA (GenBank Acc. No. AJ312240); and related sequences.

Holin can be provided in combination with an endogenous lysin, for example, a lysin encoded by an integrated bacteriophage. Holin can be provided with a recombinant lysin, for example, as supplied by a recombinant nucleic acid in the bacterium or as supplied exogenously. Moreover, the holin can be provided in the absense of any lysin.

Table 2 discloses a number of lysins available for the invention. What is contemplated is a Listeria containing a nucleic acid encoding a lysin, or a lysin deleted in its secretory or membrane-associating sequence.

TABLE 2
Nucleic acids encoding lysins.

| Lysin | Citation |
|---|---|
| Listeria murA | Carroll, et al. (2003) J. Bacteriol. 185: 6801-6808; GenBank Acc. No. |
| peptidoglycan | NC_002973 (complement of nt 2558636-2559928); GenBank Acc. No. |
| hydrolase | AM039955; GenBank Acc. No. NC003212 (complement of |
| | ny 2688314-2689606); GenBank Acc. No. NC_003210 (complement of |
| | nt 2603863-2605155). The signal peptide of murA is encoded by nt 601-757, |
| | while mature murA protein is encoded by nt 758-2361 (GenBank Acc. No. |
| | AM039955). |
| Pediococcus | Musacchio, et al. (2003) J. Appl. Microbiol. 94: 561-570); ply118 |
| peptidoglycan | L-alanoyl-D-glutamate peptidase (see, e.g., Gaeng, et al. (2000) Appl. Environ. |
| hydrolase | Microbiology 66: 2951-2958); ply511 N-acetylmuramoyl-L-alanine amidase |
| | (see, e.g., Gaeng, et al., supra). |
| p60 (also | Lenz, et al. (2003) Proc. Natl. Acad. Sci. USA 100: 12432-12437; GenBank Acc. |
| known as | Nos. DQ060360; NC_003210 (complement of nt 618932-620380); AF532220; |
| CwhA; iap | AF532218; M95579; GenBank Acc. No. AM040043; AE017262. Monk, et al. |
| gene). | (2004) Appl. Environ. Microbiology 70: 6686-6694; Bubert, et al. (1992) J. Bacteriol. |
| | 174: 8166-8171. |
| FlaA (murein | Popowska and Markiewicz (2004) Pol. J. Microbiol. 53: 237-241; Popowska |
| hydrolase). | (2004) Pol. J. Microbiol. 53: 29-34; GenBank Acc. No. AL591976 (nt |
| | 84881-85836 of segment 4/12); GenBank X65624; GenBank Acc. No. |
| | NC_003212 (nt 724183-725046). |
| Major | Ouzari, et al. (2002) J. Appl. Microbiol. 92: 812-820. |
| autolysin of | |
| Lactococcus | |
| lactis (active | |
| against | |
| Listeria). | |
| Ami (amidase; | GenBank Acc. No. AL591983 (complement of nt 240167-242920 of genome |
| autolysin). | segment 11/12); Milohanic, et al. (2001) Mol. Microbiol. 39: 1212-1224. |
| LysA. | GenBank Acc. No. AF042193; McLaughlan and Foster (1998) Microbiol. |
| | 144: 1359-1367; McLaughlan and Foster (1997) FEMS Microbiol. Lett. |
| | 152: 149-154. |
| Listerial | Reinscheid, et al. (2001) J. Bacteriol. 183: 1175-1183; GenBank Acc. No. |
| M45 peptidase | NC_003210 (complement nt 2581900-2583105), and homologous listerial |
| and | proteins, lmo2505 gene (GenBank Acc. No. AL591983). |
| homologous | |
| listerial | |
| proteins, | |
| lmo2505 gene. | |
| lin2647 gene | GenBank Acc. No. AL596173, nt 54941-56254 of segment 11/12. |
| M48 peptidase | GenBank Acc. No. AE017262. |

What is available for the invention is a nucleic acid encoding a homologous holin, or a homologous lysin, where the percent sequence identity to the parent polypeptide is normally at least 90%, more normally at least 80%, most normally at least 70%, typically at least 60%, more typically at least 50%, most typically at least 40%, conventionally at least 30%, and more conventionally at least 20% amino acid sequence identity.

What is available are nucleic acids encoding lysin enzymes active against listerial peptidoglycan (murein), for example, peptidoglycan hydrolase; murein hydrolase; endolysin; transglycosylase; endopeptidase; autolysin; lysozyme; N-acetylmuramidase; N-acetylmuramyl-L-alanine amidase (amidase); endo-β-N-acetylglucosaminidase (glucosaminidase); where the enzyme has the property of partially and/or substantially catalyzing the degradation of listerial peptidoglycan. The invention is not limited by the mechanism of murein degradation or modification, and can include mechanisms such as hydrolysis or transglycosylation (see, e.g., Carroll, et al. (2003) J. Bacteriol. 185:6801-6808; Heidrich, et al. (2002) J. Bacteriol. 184:6093-6099). Also encompassed are nucleic acids encoding lysins deleted in any secretory sequence or sorting signal that mediates cell wall attachment (see, e.g., Sabet, et al. (2005) Infection Immunity 73:6912-6922; Catt and Gregory (2005) J. Bacteriol. 187:7863-7865).

Regarding listerial constructs, in certain embodiments, nucleic acids encoding holin and/or lysin can be integrated at any position in a gene encoding a virulence factor, where integration results in an attenuating mutation. The Listeria can be engineered to contain a plurality of nucleic acids encoding a holin, where the plurality of nucleic acids can encode an identical holin, e.g., solely from listeriophage PSA, or different holins, e.g., from listeriophage and also from lambda phage. The plurality of nucleic acids can be integrated at the same locus in the listerial genome, integrated solely in the actA gene, or at different loci in the listerial genome, e.g., integrated in the actA gene and in the inlB gene. The two nucleic acids may be bicistronic.

The nucleic acid can be operably linked with a promoter that is specifically activated in a mammalian host cell, such as a prfA-activated promoter. Without implying any limitation, the promoter can be, or can be derived from, actA promoter, inlB promoter, plcA promoter, hly promoter (listeriolysin O; LLO), plcB promoter, prfA promoter, mpl promoter, and so on.

Efficacy of the holin and lysin embodiments of the invention, in mediating the processing and presentation of an antigen, can be assessed by a number of methods. These methods include, e.g., microscopy to monitor or detect antigen in a host cell's cytosol; methods of immunology sensitive to the processing or presentation of an antigen by an APC containing the Listeria (see, e.g., Porgador and Germain (1997) Immunity 6:715-726; Shastri and Gonzalez (1993) J. Immunol. 150:2724-2736); methods for measuring activation or proliferation of antigen-specific CD8⁺ T cells or CD4⁺ T cells; methods for measuring tumor size, infectious agent titer, and survival. Efficacy of the holin and lysin embodiments of the invention can also be assessed by measuring expression of the holin or lysin by the Listeria bacterium, residence of the holin within the membrane, e.g., by antibodies specific for holin, holin or lysin-mediated entry of a marker molecule from a medium into the Listeria bacterium, production of murein degradation products, and the like.

A number of Listeria-compatible promoters are available for operable linkage with a nucleic acid encoding holin, lysin, or virus-derived expression cassette. The promoter can be one that is specifically activated inside a host mammalian cell, or one that is constitutive. PrfA-dependent promoters are specifically activated in a host cell. Available prfA-dependent promoters include the prfA promoter itself, as well as actA promoter, inlB promoter, orfX promoter, orfZ promoter, uhpT promoter, and the like (Gray, et al. (2006) Infection Immunity 74:2505-2512; Chatterjee, et al. (2006) Infection Immunity 74:1323-1338). What is available are combinations of the same prfA-dependent promoters, of different prfA-dependent promoters, and of prfA-dependent and prfA-independent promoters, that is, acting in tandem and operably linked with the same ORF.

The promoter can be from a non-listerial organism (see, e.g., U.S. Pub. No. US 2005/0249748, incorporated by reference herein in its entirety). It can be a hybrid of two different promoters, it can be partially synthetic, and it can be totally synthetic, that is, having little sequence identity to a naturally occurring promoter.

What is also available are the regulatory regions, including promoters, for any of the 301 listerial genes documented to be upregulated during intracellular growth, or any of the 115 genes upregulated for growth in the cytosolic compartment (Chatterjee, et al., supra).

### III. Heterologous Polypeptides and Polynucleotides Encoding the Heterologous Polypeptides

“Heterologous polypeptides” that are encoded by polynucleotides within the Listeria and/or expressed by the Listeria are heterologous with respect to the Listeria. In certain embodiments, the heterologous polypeptides are non-listerial. In certain embodiments, the heterologous polypeptides are not found in Listeria in nature in either the genomic DNA or in any bacteriophage that has infected the Listeria. In some embodiments, the polynucleotides encoding the heterologous polypeptide(s) are recombinant.

In some embodiments, where the polynucleotide encoding the heterologous polypeptide is to be expressed within the Listeria, operably linked promoters capable of directing expression in Listeria are preferred. In some embodiments, the promoters are prokaryotic (e.g., listerial promoters such as the hly or actA promoters). In some embodiments, the polynucleotides encoding the heterologous antigen are codon-optimized for expression in Listeria (see, e.g., U.S. Patent Publication No. 2005/0249748, incorporated by reference herein in its entirety).

In some embodiments, where the polynucleotide encoding the heterologous polypeptide is to be expressed in the cytosol of an infected eukaryotic cell, such as a mammalian cell, operably linked promoters capable of directing expression in the cell are preferred. In some embodiments, the promoters are eukaryotic. In some embodiments, the polynucleotides encoding the heterologous antigen are codon-optimized for expression in the eukaryotic cell.

A variety of expression cassettes suitable for expression of antigens in Listeria are provided, e.g., in U.S. Patent Publication No. 2005/0249748, incorporated by reference herein in its entirety. Additional expression cassettes suitable for expression of heterologous polypeptides in Listeria or mammalian cells are well known in the art.

### A. Heterologous Polypeptides (e.g., Antigens)

In some embodiments, the heterologous polypeptides which are delivered or which are encoded by the nucleic acids that are delivered by the Listeria of the invention into cells (e.g., mammalian cells) comprise an antigen. In some embodiments, the antigen is a tumor antigen (e.g., a human tumor antigen), or an antigenic fragment or variant thereof. In some alternative embodiments, the antigen is an antigen from an infectious agent, or an antigenic fragment or variant thereof.

Some non-limiting examples of antigens are provided in Table 3, below.

TABLE 3
Exemplary antigens.

| | |
|---|---|
| Antigen | Reference |

Tumor antigens

| | |
|---|---|
| Mesothelin | GenBank Acc. No. NM_005823; U40434; NM_013404; BC003512 |
| | (see also, e.g., Hassan, et al. (2004) Clin. Cancer Res. 10: 3937-3942; |
| | Muminova, et al. (2004) BMC Cancer 4: 19; Iacobuzio- |
| | Donahue, et al. (2003) Cancer Res. 63: 8614-8622). |
| Wilms' tumor-1 | WT-1 isoform A (GenBank Acc. Nos. NM_000378; NP_000369). |
| associated protein | WT-1 isoform B (GenBank Acc. Nos. NM_024424; NP_077742). |
| (Wt-1), including | WT-1 isoform C (GenBank Acc. Nos. NM_024425; NP_077743). |
| isoform A; isoform B; | WT-1 isoform D (GenBank Acc. Nos. NM_024426; NP_077744). |
| isoform C; isoform D. | |
| Stratum corneum | GenBank Acc. No. NM_005046; NM_139277; AF332583. See |
| chymotryptic enzyme | also, e.g., Bondurant, et al. (2005) Clin. Cancer Res. 11: 3446-3454; |
| (SCCE), and variants | Santin, et al. (2004) Gynecol. Oncol. 94: 283-288; Shigemasa, et al. |
| thereof. | (2001) Int. J. Gynecol. Cancer 11: 454-461; Sepehr, et al. (2001) |
| | Oncogene 20: 7368-7374. |
| MHC class I | See, e.g., Groh, et al. (2005) Proc. Natl. Acad. Sci. USA 102: 6461-6466; |
| chain-related protein A | GenBank Acc. Nos. NM_000247; BC_016929; AY750850; |
| (MICA); MHC class I | NM_005931. |
| chain-related protein A | |
| (MICB). | |
| Gastrin and peptides | Harris, et al. (2004) Cancer Res. 64: 5624-5631; Gilliam, et al. |
| derived from gastrin; | (2004) Eur. J. Surg. Oncol. 30: 536-543; Laheru and Jaffee (2005) |
| gastrin/CCK-2 receptor | Nature Reviews Cancer 5: 459-467. |
| (also known as | |
| CCK-B). | |
| Glypican-3 (an antigen | GenBank Acc. No. NM_004484. Nakatsura, et al. (2003) Biochem. |
| of, e.g., hepatocellular | Biophys. Res. Commun. 306: 16-25; Capurro, et al. (2003) |
| carcinoma and | Gasteroenterol. 125: 89-97; Nakatsura, et al. (2004) Clin. Cancer |
| melanoma). | Res. 10: 6612-6621). |
| Coactosin-like protein. | Nakatsura, et al. (2002) Eur. J. Immunol. 32: 826-836; Laheru and |
| | Jaffee (2005) Nature Reviews Cancer 5: 459-467. |
| Prostate stem cell | GenBank Acc. No. AF043498; AR026974; AR302232 (see also, |
| antigen (PSCA). | e.g., Argani, et al. (2001) Cancer Res. 61: 4320-4324; Christiansen, |
| | et al. (2003) Prostate 55: 9-19; Fuessel, et al. (2003) 23: 221-228). |
| Prostate acid | Small, et al. (2000) J. Clin. Oncol. 18: 3894-3903; Altwein and |
| phosphatase (PAP); | Luboldt (1999) Urol. Int. 63: 62-71; Chan, et al. (1999) Prostate |
| prostate-specific | 41: 99-109; Ito, et al. (2005) Cancer 103: 242-250; Schmittgen, et al. |
| antigen (PSA); PSM; | (2003) Int. J. Cancer 107: 323-329; Millon, et al. (1999) Eur. Urol. |
| PSMA. | 36: 278-285. |
| Six-transmembrane | See, e.g., Machlenkin, et al. (2005) Cancer Res. 65: 6435-6442; |
| epithelial antigen of | GenBank Acc. No. NM_018234; NM_001008410; NM_182915; |
| prostate (STEAP). | NM_024636; NM_012449; BC011802. |
| Prostate carcinoma | See, e.g., Machlenkin, et al. (2005) Cancer Res. 65: 6435-6442; |
| tumor antigen-1 | GenBank Acc. No. L78132. |
| (PCTA-1). | |
| Prostate | See, e.g., Machlenkin, et al. (2005) Cancer Res. 65: 6435-6442). |
| tumor-inducing gene-1 | |
| (PTI-1). | |
| Prostate-specific gene | See, e.g., Machlenkin, et al. (2005) Cancer Res. 65: 6435-6442). |
| with homology to | |
| G protein-coupled | |
| receptor. | |
| Prostase (an antrogen | See, e.g., Machlenkin, et al. (2005) Cancer Res. 65: 6435-6442; |
| regulated serine | GenBank Acc. No. BC096178; BC096176; BC096175. |
| protease). | |
| Proteinase 3. | GenBank Acc. No. X55668. |
| Cancer-testis antigens, | GenBank Acc. No. NM_001327 (NY-ESO-1) (see also, e.g., Li, et |
| e.g., NY-ESO-1; SCP- | al. (2005) Clin. Cancer Res. 11: 1809-1814; Chen, et al. (2004) Proc. |
| 1; SSX-1; SSX-2; SSX- | Natl. Acad. Sci. USA. 101(25): 9363-9368; Kubuschok, et al. |
| 4; GAGE, CT7; CT8; | (2004) Int. J. Cancer. 109: 568-575; Scanlan, et al. (2004) Cancer |
| CT10; MAGE-1; | Immun. 4: 1; Scanlan, et al. (2002) Cancer Res. 62: 4041-4047; |
| MAGE-2; MAGE-3; | Scanlan, et al. (2000) Cancer Lett. 150: 155-164; Dalerba, et al. |
| MAGE-4; MAGE-6; | (2001) Int. J. Cancer 93: 85-90; Ries, et al. (2005) Int. J. Oncol. |
| LAGE-1. | 26: 817-824. |
| MAGE-A1, | Otte, et al. (2001) Cancer Res. 61: 6682-6687; Lee, et al. (2003) |
| MAGE-A2; | Proc. Natl. Acad. Sci. USA 100: 2651-2656; Sarcevic, et al. (2003) |
| MAGE-A3; | Oncology 64: 443-449; Lin, et al. (2004) Clin. Cancer Res. 10: 5708-5716. |
| MAGE-A4; | |
| MAGE-A6; | |
| MAGE-A9; | |
| MAGE-A10; | |
| MAGE-A12; | |
| GAGE-3/6; | |
| NT-SAR-35; BAGE; | |
| CA125. | |
| GAGE-1; GAGE-2; | De Backer, et al. (1999) Cancer Res. 59: 3157-3165; Scarcella, et al. |
| GAGE-3; GAGE-4; | (1999) Clin. Cancer Res. 5: 335-341. |
| GAGE-5; GAGE-6; | |
| GAGE-7; GAGE-8; | |
| GAGE-65; GAGE-11; | |
| GAGE-13; GAGE-7B. | |
| HIP1R; LMNA; | Scanlan, et al. (2002) Cancer Res. 62: 4041-4047. |
| KIAA1416; Seb4D; | |
| KNSL6; TRIP4; | |
| MBD2; HCAC5; | |
| MAGEA3. | |
| DAM family of genes, | Fleishhauer, et al. (1998) Cancer Res. 58: 2969-2972. |
| e.g., DAM-1; DAM-6. | |
| RCAS1. | Enjoji, et al. (2004) Dig. Dis. Sci. 49: 1654-1656. |
| RU2. | Van Den Eynde, et al. (1999) J. Exp. Med. 190: 1793-1800. |
| CAMEL. | Slager, et al. (2004) J. Immunol. 172: 5095-5102; Slager, et al. |
| | (2004) Cancer Gene Ther. 11: 227-236. |
| Colon cancer associated | Scanlan, et al. (2002) Cancer Res. 62: 4041-4047. |
| antigens, e.g., | |
| NY-CO-8; NY-CO-9; | |
| NY-CO-13; | |
| NY-CO-16; | |
| NY-CO-20; | |
| NY-CO-38; | |
| NY-CO-45; | |
| NY-CO-9/HDAC5; | |
| NY-CO-41/MBD2; | |
| NY-CO-42/TRIP4; | |
| NY-CO-95/KIAA1416; | |
| KNSL6; seb4D. | |
| N-Acetylglucosaminyl- | Dosaka-Akita, et al. (2004) Clin. Cancer Res. 10: 1773-1779. |
| tranferase V (GnT-V). | |
| Elongation factor 2 | Renkvist, et al. (2001) Cancer Immunol Immunother. 50: 3-15. |
| mutated (ELF2M). | |
| HOM-MEL-40/SSX2 | Neumann, et al. (2004) Int. J. Cancer 112: 661-668; Scanlan, et al. |
| | (2000) Cancer Lett. 150: 155-164. |
| BRDT. | Scanlan, et al. (2000) Cancer Lett. 150: 155-164. |
| SAGE; HAGE. | Sasaki, et al. (2003) Eur. J. Surg. Oncol. 29: 900-903. |
| RAGE. | See, e.g., Li, et al. (2004) Am. J. Pathol. 164: 1389-1397; Shirasawa, |
| | et al. (2004) Genes to Cells 9: 165-174. |
| MUM-1 (melanoma | Gueguen, et al. (1998) J. Immunol. 160: 6188-6194; Hirose, et al. |
| ubiquitous mutated); | (2005) Int. J. Hematol. 81: 48-57; Baurain, et al. (2000) J. Immunol. |
| MUM-2; MUM-2 Arg- | 164: 6057-6066; Chiari, et al. (1999) Cancer Res. 59: 5785-5792. |
| Gly mutation; MUM-3. | |
| LDLR/FUT fusion | Wang, et al. (1999) J. Exp. Med. 189: 1659-1667. |
| protein antigen of | |
| melanoma. | |
| NY-REN series of renal | Scanlan, et al. (2002) Cancer Res. 62: 4041-4047; Scanlan, et al. |
| cancer antigens. | (1999) Cancer Res. 83: 456-464. |
| NY-BR series of breast | Scanlan, et al. (2002) Cancer Res. 62: 4041-4047; Scanlan, et al. |
| cancer antigens, e.g., | (2001) Cancer Immunity 1: 4. |
| NY-BR-62; NY- | |
| BR-75; NY-BR-85; | |
| NY-BR-62; NY-BR-85. | |
| BRCA-1; BRCA-2. | Stolier, et al. (2004) Breast J. 10: 475-480; Nicoletto, et al. (2001) |
| | Cancer Treat Rev. 27: 295-304. |
| DEK/CAN fusion | von Lindern, et al. (1992) Mol. Cell. Biol. 12: 1687-1697. |
| protein. | |
| Ras, e.g., wild type ras, | GenBank Acc. Nos. P01112; P01116; M54969; M54968; P01111; |
| ras with mutations at | P01112; K00654. See also, e.g., GenBank Acc. Nos. M26261; |
| codon 12, 13, 59, or 61, | M34904; K01519; K01520; BC006499; NM_006270; NM_002890; |
| e.g., mutations G12C; | NM_004985; NM_033360; NM_176795; NM_005343. |
| G12D; G12R; G12S; | |
| G12V; G13D; A59T; | |
| Q61H. K-RAS; | |
| H-RAS; N-RAS. | |
| BRAF (an isoform of | Tannapfel, et al. (2005) Am. J. Clin. Pathol. 123: 256-2601; Tsao and |
| RAF). | Sober (2005) Dermatol. Clin. 23: 323-333. |
| Melanoma antigens, | GenBank Acc. No. NM_206956; NM_206955; NM_206954; |
| including HST-2 | NM_206953; NM_006115; NM_005367; NM_004988; AY148486; |
| melanoma cell | U10340; U10339; M77481. See, e g., Suzuki, et al. (1999) J. |
| antigens. | Immunol. 163: 2783-2791. |
| Survivin | GenBank Acc. No. AB028869; U75285 (see also, e.g., Tsuruma, et |
| | al. (2004) J. Translational Med. 2: 19 (11 pages); Pisarev, et al. |
| | (2003) Clin. Cancer Res. 9: 6523-6533; Siegel, et al. (2003) Br. J. |
| | Haematol. 122: 911-914; Andersen, et al. (2002) Histol. Histopathol. |
| | 17: 669-675). |
| MDM-2 | NM_002392; NM_006878 (see also, e.g., Mayo, et al. (1997) |
| | Cancer Res. 57: 5013-5016; Demidenko and Blagosklonny (2004) |
| | Cancer Res. 64: 3653-3660). |
| Methyl-CpG-binding | Muller, et al. (2003) Br. J. Cancer 89: 1934-1939; Fang, et al. (2004) |
| proteins (MeCP2; | World J. Gastreenterol. 10: 3394-3398. |
| MBD2). | |
| NA88-A. | Moreau-Aubry, et al. (2000) J. Exp. Med. 191: 1617-1624. |
| Histone deacetylases | Waltregny, et al. (2004) Eur. J. Histochem. 48: 273-290; Scanlan, et |
| (HDAC), e.g., HDAC5. | al. (2002) Cancer Res. 62: 4041-4047. |
| Cyclophilin B (Cyp-B). | Tamura, et al. (2001) Jpn. J. Cancer Res. 92: 762-767. |
| CA 15-3; CA 27.29. | Clinton, et al. (2003) Biomed. Sci. Instrum. 39: 408-414. |
| Heat shock protein | Faure, et al. (2004) Int. J. Cancer 108: 863-870. |
| Hsp70. | |
| GAGE/PAGE family, | Brinkmann, et al. (1999) Cancer Res. 59: 1445-1448. |
| e.g., PAGE-1; PAGE-2; | |
| PAGE-3; PAGE-4; | |
| XAGE-1; XAGE-2; | |
| XAGE-3. | |
| MAGE-A, B, C, and D | Lucas, et al. (2000) Int. J. Cancer 87: 55-60; Scanlan, et al. (2001) |
| families. MAGE-B5; | Cancer Immun. 1: 4. |
| MAGE-B6; | |
| MAGE-C2; | |
| MAGE-C3; MAGE-3; | |
| MAGE-6. | |
| Kinesin 2; TATA | Scanlan, et al. (2001) Cancer Immun. 30: 1-4. |
| element modulatory | |
| factor 1; tumor protein | |
| D53; NY | |
| Alpha-fetoprotein | Grimm, et al. (2000) Gastroenterol. 119: 1104-1112. |
| (AFP) | |
| SART1; SART2; | Kumamuru, et al. (2004) Int. J. Cancer 108: 686-695; Sasatomi, et |
| SART3; ART4. | al. (2002) Cancer 94: 1636-1641; Matsumoto, et al. (1998) Jpn. J. |
| | Cancer Res. 89: 1292-1295; Tanaka, et al. (2000) Jpn. J. Cancer Res. |
| | 91: 1177-1184. |
| Preferentially expressed | Matsushita, et al. (2003) Leuk. Lymphoma 44: 439-444; Oberthuer, |
| antigen of melanoma | et al. (2004) Clin. Cancer Res. 10: 4307-4313. |
| (PRAME). | |
| Carcinoembryonic | GenBank Acc. No. M29540; E03352; X98311; M17303 (see also, |
| antigen (CEA), | e.g., Zaremba (1997) Cancer Res. 57: 4570-4577; Sarobe, et al. |
| CAP1-6D enhancer | (2004) Curr. Cancer Drug Targets 4: 443-454; Tsang, et al. (1997) |
| agonist peptide. | Clin. Cancer Res. 3: 2439-2449; Fong, et al. (2001) Proc. Natl. |
| | Acad. Sci. USA 98: 8809-8814). |
| HER-2/neu. | Disis, et al. (2004) J. Clin. Immunol. 24: 571-578; Disis and Cheever |
| | (1997) Adv. Cancer Res. 71: 343-371. |
| cdk4; cdk6; p16 | Ghazizadeh, et al. (2005) Respiration 72: 68-73; Ericson, et al. |
| (INK4); Rb protein. | (2003) Mol. Cancer Res. 1: 654-664. |
| TEL; AML1; | Stams, et al. (2005) Clin. Cancer Res. 11: 2974-2980. |
| TEL/AML1. | |
| Telomerase (TERT). | Nair, et al. (2000) Nat. Med. 6: 1011-1017. |
| 707-AP. | Takahashi, et al. (1997) Clin. Cancer Res. 3: 1363-1370. |
| Annexin, e.g., | Zimmerman, et al. (2004) Virchows Arch. 445: 368-374. |
| Annexin II. | |
| BCR/ABL; BCR/ABL | Cobaldda, et al. (2000) Blood 95: 1007-1013; Hakansson, et al. |
| p210; BCR/ABL p190; | (2004) Leukemia 18: 538-547; Schwartz, et al. (2003) Semin. |
| CML-66; CML-28. | Hematol. 40: 87-96; Lim, et al. (1999) Int. J. Mol. Med. 4: 665-667. |
| BCL2; BLC6; | Iqbal, et al. (2004) Am. J. Pathol. 165: 159-166. |
| CD10 protein. | |
| CDC27 (this is a | Wang, et al. (1999) Science 284: 1351-1354. |
| melanoma antigen). | |
| Sperm protein 17 | Arora, et al. (2005) Mol. Carcinog. 42: 97-108. |
| (SP17); 14-3-3-zeta; | |
| MEMD; KIAA0471; | |
| TC21. | |
| Tyrosinase-related | GenBank Acc. No. NM_001922. (see also, e.g., Bronte, et al. |
| proteins 1 and 2 (TRP-1 | (2000) Cancer Res. 60: 253-258). |
| and TRP-2). | |
| gp100/pmel-17. | GenBank Acc. Nos. AH003567; U31798; U31799; U31807; U31799 |
| | (see also, e.g., Bronte, et al. (2000) Cancer Res. 60: 253-258). |
| TARP. | See, e.g., Clifton, et al. (2004) Proc. Natl. Acad. Sci. USA |
| | 101: 10166-10171; Virok, et al. (2005) Infection Immunity 73: 1939-1946. |
| Tyrosinase-related | GenBank Acc. No. NM_001922. (see also, e.g., Bronte, et al. |
| proteins 1 and 2 (TRP-1 | (2000) Cancer Res. 60: 253-258). |
| and TRP-2). | |
| Melanocortin 1 receptor | Salazar-Onfray, et al. (1997) Cancer Res. 57: 4348-4355; Reynolds, |
| (MC1R); MAGE-3; | et al. (1998) J. Immunol. 161: 6970-6976; Chang, et al. (2002) Clin. |
| gp100; tyrosinase; | Cancer Res. 8: 1021-1032. |
| dopachrome | |
| tautomerase (TRP-2); | |
| MART-1. | |
| MUC-1; MUC-2. | See, e.g., Davies, et al. (1994) Cancer Lett. 82: 179-184; Gambus, et |
| | al. (1995) Int. J. Cancer 60: 146-148; McCool, et al. (1999) |
| | Biochem. J. 341: 593-600. |
| Spas-1. | U.S. Published Pat. Appl. No. 20020150588 of Allison, et al. |
| CASP-8; FLICE; | Mandruzzato, et al. (1997) J. Exp. Med. 186: 785-793. |
| MACH. | |
| CEACAM6; CAP-1. | Duxbury, et al. (2004) Biochem. Biophys. Res. Commun. 317: 837-843; |
| | Morse, et al. (1999) Clin. Cancer Res. 5: 1331-1338. |
| HMGB1 (a DNA | Brezniceanu, et al. (2003) FASEB J. 17: 1295-1297. |
| binding protein and | |
| cytokine). | |
| ETV6/AML1. | Codrington, et al. (2000) Br. J. Haematol. 111: 1071-1079. |
| Mutant and wild type | Clements, et al. (2003) Clin. Colorectal Cancer 3: 113-120; |
| forms of adenomatous | Gulmann, et al. (2003) Appl. Immunohistochem. Mol. Morphol. |
| polyposis coli (APC); | 11: 230-237; Jungck, et al. (2004) Int. J. Colorectal. Dis. 19: 438-445; |
| beta-catenin; c-met; | Wang, et al. (2004) J. Surg. Res. 120: 242-248; Abutaily, et al. |
| p53; E-cadherin; | (2003) J. Pathol. 201: 355-362; Liang, et al. (2004) Br. J. Surg. |
| cyclooxygenase-2 | 91: 355-361; Shirakawa, et al. (2004) Clin. Cancer Res. 10: 4342-4348. |
| (COX-2). | |
| Renal cell carcinoma | Mulders, et al. (2003) Urol. Clin. North Am. 30: 455-465; Steffens, |
| antigen bound by mAB | et al. (1999) Anticancer Res. 19: 1197-1200. |
| G250. | |

Francisella tularensis antigens

| | |
|---|---|
| Francisella tularensis | Complete genome of subspecies Schu S4 (GenBank Acc. No. |
| A and B. | AJ749949); of subspecies Schu 4 (GenBank Acc. No. NC_006570). |
| | Outer membrane protein (43 kDa) Bevanger, et al. (1988) J. Clin. |
| | Microbiol. 27: 922-926; Porsch-Ozcurumez, et al. (2004) Clin. |
| | Diagnostic. Lab. Immunol. 11: 1008-1015). Antigenic components |
| | of F. tularensis include, e.g., 80 antigens, including 10 kDa and |
| | 60 kDa chaperonins (Havlasova, et al. (2002) Proteomics 2: 857-86), |
| | nucleoside diphosphate kinase, isocitrate dehydrogenase, |
| | RNA-binding protein Hfq, the chaperone ClpB (Havlasova, et al. |
| | (2005) Proteomics 5: 2090-2103). See also, e.g., Oyston and Quarry |
| | (2005) Antonie Van Leeuwenhoek 87: 277-281; Isherwood, et al. |
| | (2005) Adv. Drug Deliv. Rev. 57: 1403-1414; Biagini, et al. (2005) |
| | Anal. Bioanal. Chem. 382: 1027-1034. |

Malarial antigens

| | |
|---|---|
| Circumsporozoite | See, e.g., Haddad, et al. (2004) Infection Immunity 72: 1594-1602; |
| protein (CSP); SSP2; | Hoffman, et al. (1997) Vaccine 15: 842-845; Oliveira-Ferreira and |
| HEP17; Exp-1 | Daniel-Ribeiro (2001) Mem. Inst. Oswaldo Cruz, Rio de Janeiro |
| orthologs found in | 96: 221-227. CSP (see, e.g., GenBank Acc. No. AB121024). SSP2 |
| P. falciparum, and | (see, e.g., GenBank Acc. No. AF249739). LSA-1 (see, e.g., |
| LSA-1. | GenBank Acc. No. Z30319). |
| Ring-infected | See, e.g., Stirnadel, et al. (2000) Int. J. Epidemiol. 29: 579-586; |
| erythrocyte survace | Krzych, et al. (1995) J. Immunol. 155: 4072-4077. See also, Good, |
| protein (RESA); | et al. (2004) Immunol. Rev. 201: 254-267; Good, et al. (2004) Ann. |
| merozoite surface | Rev. Immunol. 23: 69-99. MSP2 (see, e.g., GenBank Acc. No. |
| protein 2 (MSP2); | X96399; X96397). MSP1 (see, e.g., GenBank Acc. No. X03371). |
| Spf66; merozoite | RESA (see, e.g., GenBank Acc. No. X05181; X05182). |
| surface | |
| protein 1(MSP1); | |
| 195A; BVp42. | |
| Apical membrane | See, e.g., Gupta, et al. (2005) Protein Expr. Purif. 41: 186-198. |
| antigen 1 (AMA1). | AMA1 (see, e.g., GenBank Acc. No. A′13; AJ494905; AJ490565). |

Viruses and viral antigens

| | |
|---|---|
| Hepatitis A | GenBank Acc. Nos., e.g., NC_001489; AY644670; X83302; |
| | K02990; M14707. |
| Hepatitis B | Complete genome (see, e.g., GenBank Acc. Nos. AB214516; |
| | NC_003977; AB205192; AB205191; AB205190; AJ748098; |
| | AB198079; AB198078; AB198076; AB074756). |
| Hepatitis C | Complete genome (see, e.g., GenBank Acc. Nos. NC_004102; |
| | AJ238800; AJ238799; AJ132997; AJ132996; AJ000009; D84263). |
| Hepatitis D | GenBank Acc. Nos, e.g. NC_001653; AB118847; AY261457. |
| Human papillomavirus, | See, e.g., Trimble, et al. (2003) Vaccine 21: 4036-4042; Kim, et al. |
| including all 200+ | (2004) Gene Ther. 11: 1011-1018; Simon, et al. (2003) Eur. J. |
| subtypes (classed in | Obstet. Gynecol. Reprod. Biol. 109: 219-223; Jung, et al. (2004) J. |
| 16 groups), such as the | Microbiol. 42: 255-266; Damasus-Awatai and Freeman-Wang |
| high risk subtypes 16, | (2003) Curr. Opin. Obstet. Gynecol. 15: 473-477; Jansen and Shaw |
| 18, 30, 31, 33, 45. | (2004) Annu. Rev. Med. 55: 319-331; Roden and Wu (2003) |
| | Expert Rev. Vaccines 2: 495-516; de Villiers, et al. (2004) Virology |
| | 324: 17-24; Hussain and Paterson (2005) Cancer Immunol. |
| | Immunother. 54: 577-586; Molijn, et al. (2005) J. Clin. Virol. 32 |
| | (Suppl. 1) S43-S51. GenBank Acc. Nos. AY686584; AY686583; |
| | AY686582; NC_006169; NC_006168; NC_006164; NC_001355; |
| | NC_001349; NC_005351; NC_001596). |
| Human T-cell | See, e.g., Capdepont, et al. (2005) AIDS Res. Hum. Retrovirus |
| lymphotropic virus | 21: 28-42; Bhigjee, et al. (1999) AIDS Res. Hum. Restrovirus |
| (HTLV) types I and II, | 15: 1229-1233; Vandamme, et al. (1998) J. Virol. 72: 4327-4340; |
| including the | Vallejo, et al. (1996) J. Acquir. Immune Defic. Syndr. Hum. |
| HTLV type I subtypes | Retrovirol. 13: 384-391. HTLV type I (see, e.g., GenBank Acc. |
| Cosmopolitan, Central | Nos. AY563954; AY563953. HTLV type II (see, e.g., GenBank |
| African, and | Acc. Nos. L03561; Y13051; AF139382). |
| Austro-Melanesian, and | |
| the HTLV type II | |
| subtypes IIa, IIb, IIc, | |
| and IId. | |
| Coronaviridae, | See, e.g., Brian and Baric (2005) Curr. Top. Microbiol. Immunol. |
| including | 287: 1-30; Gonzalez, et al. (2003) Arch. Virol. 148: 2207-2235; |
| Coronaviruses, such as | Smits, et al. (2003) J. Virol. 77: 9567-9577; Jamieson, et al. (1998) |
| SARS-coronavirus | J. Infect. Dis. 178: 1263-1269 (GenBank Acc. Nos. AY348314; |
| (SARS-CoV), and | NC_004718; AY394850). |
| Toroviruses. | |
| Rubella virus. | GenBank Acc. Nos. NC_001545; AF435866. |
| Mumps virus, including | See, e.g., Orvell, etal. (2002) J. Gen. Virol. 83: 2489-2496. See, |
| the genotypes A, C, D, | e.g., GenBank Acc. Nos. AY681495; NC_002200; AY685921; |
| G, H, and I. | AF201473. |
| Coxsackie virus A | See, e.g., Brown, et al. (2003) J. Virol. 77: 8973-8984. GenBank |
| including the serotypes | Acc. Nos. AY421768; AY790926: X67706. |
| 1, 11, 13, 15, 17, 18, | |
| 19, 20, 21, 22, and 24 | |
| (also known as Human | |
| enterovirus C; HEV-C). | |
| Coxsackie virus B, | See, e.g., Ahn, et al. (2005) J. Med. Virol. 75: 290-294; Patel, et al. |
| including subtypes 1-6. | (2004) J. Virol. Methods 120: 167-172; Rezig, et al. (2004) J. Med. |
| | Virol. 72: 268-274. GenBank Acc. No. X05690. |
| Human enteroviruses | See, e.g., Oberste, et al. (2004) J. Virol. 78: 855-867. Human |
| including, e.g., human | enterovirus A (GenBank Acc. Nos. NC_001612); human |
| enterovirus A (HEV-A, | enterovirus B (NC_001472); human enterovirus C (NC_001428); |
| CAV2 to CAV8, | human enterovirus D (NC_001430). Simian enterovirus A |
| CAV10, CAV12, | (GenBank Acc. No. NC_003988). |
| CAV14, CAV16, and | |
| EV71) and also | |
| including HEV-B | |
| (CAV9, CBV1 to | |
| CBV6, E1 to E7, E9, | |
| E11 to E21, E24 to | |
| E27, E29 to E33, and | |
| EV69 and E73), as well | |
| as HEV. | |
| Polioviruses including | See, e.g., He, et al. (2003) J. Virol. 77: 4827-4835; Hahsido, et al. |
| PV1, PV2, and PV3. | (1999) Microbiol. Immunol. 43: 73-77. GenBank Acc. No. |
| | AJ132961 (type 1); AY278550 (type 2); X04468 (type 3). |
| Viral encephalitides | See, e.g., Hoke (2005) Mil. Med. 170: 92-105; Estrada-Franco, et al. |
| viruses, including | (2004) Emerg. Infect. Dis. 10: 2113-2121; Das, et al. (2004) |
| equine encephalitis, | Antiviral Res. 64: 85-92; Aguilar, et al. (2004) Emerg. Infect. Dis. |
| Venezuelan equine | 10: 880-888; Weaver, et al. (2004) Arch. Virol. Suppl. 18: 43-64; |
| encephalitis (VEE) | Weaver, et al. (2004) Annu. Rev. Entomol. 49: 141-174. Eastern |
| (including subtypes IA, | equine encephalitis (GenBank Acc. No. NC_003899; AY722102); |
| IB, IC, ID, IIIC, IIID), | Western equine encephalitis (NC_003908). |
| Eastern equine | |
| encephalitis (EEE), | |
| Western equine | |
| encephalitis (WEE), | |
| St. Louis encephalitis, | |
| Murray Valley | |
| (Australian) | |
| encephalitis, Japanese | |
| encephalitis, and | |
| tick-born encephalitis. | |
| Human herpesviruses, | See, e.g., Studahl, et al. (2000) Scand. J. Infect. Dis. 32: 237-248; |
| including | Padilla, et al. (2003) J. Med. Virol. 70 (Suppl. 1) S103-S110; |
| cytomegalovirus | Jainkittivong and Langlais (1998) Oral Surg. Oral Med. 85: 399-403. |
| (CMV), Epstein-Barr | GenBank Nos. NC_001806 (herpesvirus 1); NC_001798 |
| virus (EBV), human | (herpesvirus 2); X04370 and NC_001348 (herpesvirus 3); |
| herpesvirus-1 (HHV-1), | NC_001345 (herpesvirus 4); NC_001347 (herpesvirus 5); X83413 |
| HHV-2, HHV-3, | and NC_000898 (herpesvirus 6); NC_001716 (herpesvirus 7). |
| HHV-4, HHV-5, | Human herpesviruses types 6 and 7 (HHV-6; HHV-7) are disclosed |
| HHV-6, HHV-7, | by, e.g., Padilla, et al. (2003) J. Med. Virol. 70 (Suppl. 1)S103-S110. |
| HHV-8, herpes B virus, | Human herpesvirus 8 (HHV-8), including subtypes A-E, are |
| herpes simplex virus | disclosed in, e.g., Treurnicht, et al. (2002) J. Med. Virul. 66: 235-240. |
| types 1 and 2 (HSV-1, | |
| HSV-2), and varicella | |
| zoster virus (VZV). | |
| HIV-1 including group | See, e.g., Smith, et al. (1998) J. Med. Virol. 56: 264-268. See also, |
| M (including subtypes | e.g., GenBank Acc. Nos. DQ054367; NC_001802; AY968312; |
| A to J) and group O | DQ011180; DQ011179; DQ011178; DQ011177; AY588971; |
| (including any | AY588970; AY781127; AY781126; AY970950; AY970949; |
| distinguishable | AY970948; X61240; AJ006287; AJ508597; and AJ508596. |
| subtypes) (HIV-2, | |
| including subtypes | |
| A-E. | |
| Epstein-Barr virus | See, e.g., Peh, et al. (2002) Pathology 34: 446-450. |
| (EBV), including | Epstein-Barr virus strain B95-8 (GenBank Acc. No. V01555). |
| subtypes A and B. | |
| Reovirus, including | See, e.g., Barthold, et al. (1993) Lab. Anim. Sci. 43: 425-430; Roner, |
| serotypes and strains 1, | et al. (1995) Proc. Natl. Acad. Sci. USA 92: 12362-12366; Kedl, et |
| 2, and 3, type 1 Lang, | al. (1995) J. Virol. 69: 552-559. GenBank Acc. No. K02739 |
| type 2 Jones, and type 3 | (sigma-3 gene surface protein). |
| Dearing. | |
| Cytomegalovirus | See, e.g., Chern, et al. (1998) J. Infect. Dis. 178: 1149-1153; Vilas |
| (CMV) subtypes | Boas, et al. (2003) J. Med. Virol. 71: 404-407; Trincado, et al. |
| include CMV subtypes | (2000) J. Med. Virol. 61: 481-487. GenBank Acc. No. X17403. |
| I-VII. | |
| Rhinovirus, including | Human rhinovirus 2 (GenBank Acc. No. X02316); Human |
| all serotypes. | rhinovirus B (GenBank Acc. No. NC_001490); Human rhinovirus |
| | 89 (GenBank Acc. No. NC_001617); Human rhinovirus 39 |
| | (GenBank Acc. No. AY751783). |
| Adenovirus, including | AY803294; NC_004001; AC_000019; AC_000018; AC_000017; |
| all serotypes. | AC_000015; AC_000008; AC_000007; AC_000006; AC_000005; |
| | AY737798; AY737797; NC_003266; NC_002067; AY594256; |
| | AY594254; AY875648; AJ854486; AY163756; AY594255; |
| | AY594253; NC_001460; NC_001405; AY598970; AY458656; |
| | AY487947; NC_001454; AF534906; AY45969; AY128640; |
| | L19443; AY339865; AF532578. |
| Varicella-zoster virus, | See, e.g., Loparev, et al. (2004) J. Virol. 78: 8349-8358; Carr, et al. |
| including strains and | (2004) J. Med. Virol. 73: 131-136; Takayama and Takayama (2004) |
| genotypes Oka, Dumas, | J. Clin. Virol. 29: 113-119. |
| European, Japanese, | |
| and Mosaic. | |
| Filoviruses, including | See, e.g., Geisbert and Jahrling (1995) Virus Res. 39: 129-150; |
| Marburg virus and | Hutchinson, et al. (2001) J. Med. Virol. 65: 561-566. Marburg virus |
| Ebola virus, and strains | (see, e.g., GenBank Acc. No. NC_001608). Ebola virus (see, e.g., |
| such as Ebola-Sudan | GenBank Acc. Nos. NC_006432; AY769362; NC_002549; |
| (EBO-S), Ebola-Zaire | AF272001; AF086833). |
| (EBO-Z), and | |
| Ebola-Reston (EBO-R). | |
| Arenaviruses, including | Junin virus, segment S (GenBank Acc. No. NC_005081); Junin |
| lymphocytic | virus, segment L (GenBank Acc. No. NC_005080). |
| choriomeningitis | |
| (LCM) virus, Lassa | |
| virus, Junin virus, and | |
| Machupo virus. | |
| Rabies virus. | See, e.g., GenBank Acc. Nos. NC_001542; AY956319; AY705373; |
| | AF499686; AB128149; AB085828; AB009663. |
| Arboviruses, including | Dengue virus type 1 (see, e.g., GenBank Acc. Nos. AB195673; |
| West Nile virus, | AY762084). Dengue virus type 2 (see, e.g., GenBank Acc. Nos. |
| Dengue viruses 1 to 4, | NC_001474; AY702040; AY702039; AY702037). Dengue virus |
| Colorado tick fever | type 3 (see, e.g., GenBank Acc. Nos. AY923865; AT858043). |
| virus, Sindbis virus, | Dengue virus type 4 (see, e.g., GenBank Acc. Nos. AY947539; |
| Togaviraidae, | AY947539; AF326573). Sindbis virus (see, e.g., GenBank Acc. |
| Flaviviridae, | Nos. NC_001547; AF429428; J02363; AF103728). West Nile virus |
| Bunyaviridae, | (see, e.g., GenBank Acc. Nos. NC_001563; AY603654). |
| Reoviridae, | |
| Rhabdoviridae, | |
| Orthomyxoviridae, and | |
| the like. | |
| Poxvirus including | Viriola virus (see, e.g., GenBank Acc. Nos. NC_001611; Y16780; |
| orthopoxvirus (variola | X72086; X69198). |
| virus, monkeypox | |
| virus, vaccinia virus, | |
| cowpox virus), | |
| yatapoxvirus (tanapox | |
| virus, Yaba monkey | |
| tumor virus), | |
| parapoxvirus, and | |
| molluscipoxvirus. | |
| Yellow fever. | See, e.g., GenBank Acc. No. NC_002031; AY640589; X03700. |
| Hantaviruses, including | See, e.g., Elgh, et al. (1997) J. Clin. Microbiol. 35: 1122-1130; |
| serotypes Hantaan | Sjolander, et al. (2002) Epidemiol. Infect. 128: 99-103; Zeier, et al. |
| (HTN), Seoul (SEO), | (2005) Virus Genes 30: 157-180. GenBank Acc. No. NC_005222 |
| Dobrava (DOB), Sin | and NC_005219 (Hantavirus). See also, e.g., GenBank Acc. Nos. |
| Nombre (SN), Puumala | NC_005218; NC_005222; NC_005219. |
| (PUU), and | |
| Dobrava-like Saaremaa | |
| (SAAV). | |
| Flaviviruses, including | See, e.g., Mukhopadhyay, et al. (2005) Nature Rev. Microbiol. 3: 13-22. |
| Dengue virus, Japanese | GenBank Acc. Nos NC_001474 and AY702040 (Dengue). |
| encephalitis virus, West | GenBank Acc. Nos. NC_001563 and AY603654. |
| Nile virus, and yellow | |
| fever virus. | |
| Measles virus. | See, e.g., GenBank Acc. Nos. AB040874 and AY486084. |
| Human | Human parainfluenza virus 2 (see, e.g., GenBank Acc. Nos. |
| parainfluenzaviruses | AB176531; NC003443). Human parainfluenza virus 3 (see, e.g., |
| (HPV), including HPV | GenBank Acc. No. NC_001796). |
| types 1-56. | |
| Influenza virus, | Influenza nucleocapsid (see, e.g., GenBank Acc. No. AY626145). |
| including influenza | Influenza hemagglutinin (see, e.g., GenBank Acc. Nos. AY627885; |
| virus types A, B, and C. | AY555153). Influenza neuraminidase (see, e.g., GenBank Acc. |
| | Nos. AY555151; AY577316). Influenza matrix protein 2 (see, e.g., |
| | GenBank Acc. Nos. AY626144(. Influenza basic protein 1 (see, |
| | e.g., GenBank Acc. No. AY627897). Influenza polymerase acid |
| | protein (see, e.g., GenBank Acc. No. AY627896). Influenza |
| | nucleoprotein (see, e.g., GenBank Acc. Nno. AY627895). |
| Influenza A virus | Hemagglutinin of H1N1 (GenBank Acc. No. S67220). Influenza A |
| subtypes, e.g., swine | virus matrix protein (GenBank Acc. No. AY700216). Influenza |
| viruses (SIV): H1N1 | virus A H5H1 nucleoprotein (GenBank Acc. No. AY646426). |
| influenza A and swine | H1N1 haemagglutinin (GenBank Acc. No. D00837). See also, |
| influenza virus. | GenBank Acc. Nos. BD006058; BD006055; BD006052. See also, |
| | e.g., Wentworth, et al. (1994) J. Virol. 68: 2051-2058; Wells, et al. |
| | (1991) J.A.M.A. 265: 478-481. |
| Respiratory syncytial | Respiratory syncytial virus (RSV) (see, e.g., GenBank Acc. Nos. |
| virus (RSV), including | AY353550; NC_001803; NC001781). |
| subgroup A and | |
| subgroup B. | |
| Rotaviruses, including | Human rotavirus C segment 8 (GenBank Acc. No. AJ549087); |
| human rotaviruses A to | Human rotavirus G9 strain outer capsid protein (see, e.g., GenBank |
| E, bovine rotavirus, | Acc. No. DQ056300); Human rotavirus B strain non-structural |
| rhesus monkey | protein 4 (see, e.g., GenBank Acc. No. AY548957); human |
| rotavirus, and | rotavirus A strain major inner capsid protein (see, e.g., GenBank |
| human-RVV | Acc. No. AY601554). |
| reassortments. | |
| Polyomavirus, | See, e.g., Engels, et al. (2004) J. Infect. Dis. 190: 2065-2069; |
| including simian | Vilchez and Butel (2004) Clin. Microbiol. Rev. 17: 495-508; |
| virus 40 (SV40), JC | Shivapurkar, et al. (2004) Cancer Res. 64: 3757-3760; Carbone, et |
| virus (JCV) and BK | al. (2003) Oncogene 2: 5173-5180; Barbanti-Brodano, et al. (2004) |
| virus (BKV). | Virology 318: 1-9) (SV40 complete genome in, e.g., GenBank Acc. |
| | Nos. NC_001669; AF168994; AY271817; AY271816; AY120890; |
| | AF345344; AF332562). |
| Coltiviruses, including | Attoui, et al. (1998) J. Gen. Virol. 79: 2481-2489. Segments of |
| Colorado tick fever | Eyach virus (see, e.g., GenBank Acc. Nos. AF282475; AF282472; |
| virus, Eyach virus. | AF282473; AF282478; AF282476; NC_003707; NC_003702; |
| | NC_003703; NC_003704; NC_003705; NC_003696; NC_003697; |
| | NC_003698; NC_003699; NC_003701; NC_003706; NC_003700; |
| | AF282471; AF282477). |
| Calciviruses, including | Snow Mountain virus (see, e.g., GenBank Acc. No. AY134748). |
| the genogroups | |
| Norwalk, Snow | |
| Mountain group | |
| (SMA), and Saaporo. | |
| Parvoviridae, including | See, e.g., Brown (2004) Dev. Biol. (Basel) 118: 71-77; Alvarez- |
| dependovirus, | Lafuente, et al. (2005) Ann. Rheum. Dis. 64: 780-782; Ziyaeyan, et |
| parvovirus (including | al. (2005) Jpn. J. Infect. Dis. 58: 95-97; Kaufman, et al. (2005) |
| parvovirus B19), and | Virology 332: 189-198. |
| erythrovirus. | |

Each of the references, GenBank Acc. Nos., and the nucleic acids, peptides, and polypeptides cited in this table are hereby incorporated herein by reference in their entirety.

In some embodiments, the antigen is mesothelin, prostate stem cell antigen (PSCA), hepatitis B antigen, or hepatitis C antigen, or an antigenic fragment or variant thereof. In some embodiments, the antigen is mesothelin (e.g., human mesothelin) deleted of its signal peptide and/or GPI (glycosylphosphatidylinositol) anchor.

The antigenic fragment may be of any length, but is most typically at least about 6 amino acids, at least about 9 amino acids, at least about 12 amino acids, at least about 20 amino acids, at least about 30 amino acids, at least about 50 amino acids, or at least about 100 amino acids. An antigenic fragment of an antigen comprises at least one epitope from the antigen. In some embodiments, the epitope is a MHC class I epitope. In other embodiments, the epitope is a MHC class II epitope. In some embodiments, the epitope is a CD4+ T-cell epitope. In other embodiments, the epitope is a CD8+ T-cell epitope.

A variety of algorithms and software packages useful for predicting antigenic regions (including epitopes) within proteins are available to those skilled in the art. For instance, algorthims that can be used to select epitopes that bind to MHC class I and class II molecules are publicly available. For instance, the publicly available “SYFPEITHI” algorithm can be used to predict MHC-binding peptides (Rammensee et al. (1999) Immunogenetics 50:213-9). For other examples of publicly available algorithms, see the following references: Parker et al. (1994) J. Immunol 152:163-75; Singh and Raghava (2001) Bioinformatics 17:1236-1237; Singh and Raghava (2003) Bioinformatics 19:1009-1014; Mallios (2001) Bioinformatics 17:942-8; Nielsen et al. (2004) Bioinformatics 20:1388-97; Donnes et al. (2002) BMC Bioinformatics 3:25; Bhasin, et al. (2004) Vaccine 22:3195-204; Guan et al. (2003) Nucleic Acids Res 31:3621-4; Reche et al. (2002) Hum. Immunol. 63:701-9; Schirle et al. (2001) J. Immunol Methods 257:1-16; Nussbaum et al. (2001) Immunogenetics (2001) 53:87-94; Lu et al. (2000) Cancer Res. 60:5223-7. See also, e.g., Vector NTI® Suite (Informax, Inc, Bethesda, Md.), GCG Wisconsin Package (Accelrys, Inc., San Diego, Calif.), Welling, et al. (1985) FEBS Lett. 188:215-218, Parker, et al. (1986) Biochemistry 25:5425-5432, Van Regenmortel and Pellequer (1994) Pept. Res. 7:224-228, Hopp and Woods (1981) PNAS 78:3824-3828, and Hopp (1993) Pept. Res. 6:183-190. Some of the algorthims or software packages discussed in the references listed above in this paragraph are directed to the prediction of MHC class I and/or class II binding peptides or epitopes, others to identification of proteasomal cleavage sites, and still others to prediction of antigenicity based on hydrophilicity.

Once a candidate antigenic fragment believed to contain at least one epitope of the desired nature has been identified, the polynucleotide sequence encoding that sequence can be incorporated into an expression cassette and introduced into a Listeria vaccine vector or other bacterial vaccine vector. The immunogenicity of the antigenic fragment can then be confirmed by assessing the immune response generated by the Listeria or other bacteria expressing the fragments. Standard immunological assays such as ELISPOT assays, Intracellular Cytokine Staining (ICS) assay, cytotoxic T-cell activity assays, or the like, can be used to verify that the fragment of the antigen chosen maintains the desired imunogenicity. In addition, the anti-tumor efficacy of the Listeria and/or bacterial vaccines can also be assessed using animal models (e.g., implantation of CT26 murine colon cells expressing the antigen fragment in mice, followed by vaccination of the mice with the candidate vaccine and observation of effect on tumor size, metastasis, survival, etc. relative to controls and/or the full-length antigen).

In addition, large databases containing epitope and/or MHC ligand information using for identifying antigenic fragments are publicly available. See, e.g., Brusic et al. (1998) Nucleic Acids Res. 26:368-371; Schonbach et al. (2002) Nucleic Acids Research 30:226-9; and Bhasin et al. (2003) Bioinformatics 19:665-666; and Rammensee et al. (1999) Immunogenetics 50:213-9.

The amino acid sequence of an antigenic variant has at least about 60%, at least about 70%, at least about 80%, at least about 90%, at least about 95%, or at least about 98% identity to the original antigen.

In some embodiments, the antigenic variant is a conservative variant that has at least about 80% identity to the original antigen and the substitutions between the sequence of the antigenic variant and the original antigen are conservative amino acid substitutions. The following substitutions are considered conservative amino acid substitutions: valine, isoleucine, or leucine are substituted for alanine; lysine, glutamine, or asparagine are substituted for arginine; glutamine, histidine, lysine, or arginine are substituted for asparagine; glutamic acid is substituted for aspartic acid; serine is substituted for cysteine; asparagine is substituted for glutamine; aspartic acid is substituted for glutamic acid; proline or alanine is substituted for glycine; asparagine, glutamine, lysine or arginine is substituted for histidine; leucine, valine, methionine, alanine, phenylalanine, or norleucine is substituted for isoleucine; norleucine, isoleucine, valine, methionine, alanine, or phenylalanine is substituted for leucine; arginine, glutamine, or asparagine is substituted for lysine; leucine, phenylalanine, or isoleucine is substituted for methionine; leucine, valine, isoleucine, alanine, or tyrosine is substituted for phenylalanine; alanine is substituted for proline; threonine is substituted for serine; serine is substituted for threonine; tyrosine or phenylalanine is substituted for tryptophan; tryptophan, phenylalanine, threonine, or serine is substituted for tyrosine; tryptophan, phenylalanine, threonine, or serine is substituted for tyrosine; isoleucine, leucine, methionine, phenylalanine, alanine, or norleucine is substituted for valine. In some embodiments, the antigenic variant is a convervative variant that has at least about 90% or at least about 95%identity to the original antigen.

“Percent (%) sequence identity” (or, alternatively, the “percent (%) identical”), as used herein with respect to amino acid sequences, refers to the percentage of amino acid residues in a candidate sequence (such as a variant of an antigen) that is identical to the amino acid residues in a specific reference sequence (such as in a specific antigen sequence), after aligning the sequences and introducing gaps, if necessary, to achieve the maximum percent sequence identity, and not considering any conservative substitutions a part of the sequence identity. Alignment for purposes of determining percent amino acid sequence identity can be achieved in various ways that are within the skill in the art, for instance, using any of the publicly available algorithms and/or computer software for sequence alignment, or by inspection. Those skilled in the art can determine appropriate parameters for measuring alignment, including any algorithms needed to achieve maximal alignment over the full-length of the sequences being compared. The % sequence identity of a given amino acid sequence A to a given amino acid sequence B is calculated as follows: 100 times the fraction X/Y, where X is the number of identical matches in the optimal alignment of the A and B sequences, and where Y is the total number of amino acid residues in B.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Alternatively, the % (amino acid) sequence identity may be obtained using one of the publicly available BLAST or BLAST-2 programs. The WU-BLAST-2 computer program (Altschul et al., Methods in Enzymology 266:460-480 (1996)). Percent (amino acid) sequence identity may also be determined using the sequence comparison program NCBI-BLAST2 (Altschul et al., Nucleic Acids Res. 25:3389-3402 (1997)). The BLAST program is based on the alignment method of Karlin and Altschul. Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997).

### B. Self-Replicating RNAs and Virus-Derived Nucleic Acid Expression Cassettes

In some embodiments, the Listeria of the invention comprise a polynucleotide encoding an RNA such as a self-replicating RNA and/or a virus-derived nucleic acid expression cassette. As used herein, a “self-replicating RNA” encompasses an RNA sequence or molecule that contains all of the genetic information necessary to encode all proteins necessary for self-amplification or self-replication in an appropriate environment (e.g., in the cytosol of a mammalian host cell.) In certain embodiments, the self-replicating RNA sequences are derived from viruses, such as ssRNA positive-strand virus. The RNAs may be generated from the polynucleotide in the Listeria or in the cytosol of an infected cell following holin-dependent externalization. The RNAs encode a heterologous polypeptide which, in some embodiments, is expressed from the RNA within the Listeria. In some embodiments the heterologous polypeptide is instead translated in the cytosol of an infected cell.

The invention provides a polynucleotide encoding an expression cassette derived from a ssRNA positive-strand virus (no DNA stage). Also provided is a Listeria bacterium containing a polynucleotide, for example, a genomic or plasmid-based nucleic acid, encoding an expression cassette derived from a ssRNA positive-strand virus (no DNA stage). The expression cassette can be from, or derived from, a member of the Togaviridae; Flaviviridae; Caliciviridae; Leviviridae; Picornaviridae; Tetraviridae; Tobamovirus; Nodaviridae; Astroviridae; Barnaviridae; Nidovirales; Dicistroviridae; Iflavirus; or Hepeviridae. In some embodiments, the expression cassette is from, or derived from, a member of the Picornaviridae. For example, it can be a Enterovirus; Hepatovirus; Cardiovirus; Aphthovirus; Rhinovirus; Teschovirus; Parechovirus; Kobuvirus; or Erbovirus. Typically, the Enterovirus-derived expression cassette is from a Bovine enterovirus; Coxsackievirus; Echovirus; Porcine enterovirus B; Sheep enterovirus; Porcine enterovirus A; Human enterovirus A; Human enterovirus B; Human enterovirus C; Human enterovirus D; Poliovirus; or Simian enterovirus A. The Nidoviridae virus-derived expression cassette can be from, or derived from, Coronaviridae, Arteriviridae, or Roniviridae. Coronaviridae-derived expression cassettes have been described (see, e.g., Verheije, et al. (2006) J. Virol. 80:1250-1260; Sola, et al. (2003) J. Virol. 77:4357-4369). Sometimes, the Flaviviridae-derived expression cassette can be from a Flavivirus, Pestivirus, or Hepacivirus. In certain embodiments, the Hepatovirus-derived expression cassette is from Hepatitis A virus or Avian encephalomyelitis virus. Sometimes the Aphthovirus is Foot-and-mouth disease virus or Equine rhinitis A virus.

In some embodiments, the RNA generated within the Listeria or released from the Listeria comprises an expression cassette derived from an ssRNA positive-strand virus. In some embodiments, the virus is selected from the group consisting of togavirus, flavivirus, pestivirus, and picornavirus. In some embodiments, the expression cassette is derived from a togavirus. For instance, the RNA may comprise an alphavirus replicon that expresses the heterologous polypeptide. The alphavirus replicon may be derived from Sindbis virus, Venezuelan Equine Encephalitis (VEE) virus, or Semliki Forest virus (SFV). In some embodiments, the RNA comprises a flavivirus replicon (e.g., derived from the Kunjin virus) that expresses the heterologous polypeptide. Alternatively, the RNA may comprise a picornavirus replicon (e.g., a replicon derived from Encephalomycocarditis (EMCV) virus, poliovirus, or coxsackie virus) that expresses the heterologous polypeptide. For instance, the expression cassette can be derived from the Encephalomycocarditis (EMCV) virus.

The invention, in some aspects, provides a Listeria bacterium containing a polynucleotide encoding a togavirus-derived expression cassette. The polynucleotide, in some embodiments, is integrated into the listerial genome, where integration can be mediated by site-specific recombination or by homologous recombination. In some aspects, the polynucleotide is integrated by homologous recombination within a virulence factor gene, while in other aspects, the polynucleotide is not integrated within a virulence factor gene. The polynucleotide encoding the cassette is operably linked with a listerial promoter, or a synthetic promoter active in Listeria. In some aspects, the promoter, e.g., a prfA-dependent promoter, is specifically activated in the environment of the host cell.

In certain embodiments, the togavirus-derived expression cassette encodes an RNA synthesized inside the Listeria bacterium, where the RNA is subsequently released from the bacterium to the host cell's cytoplasm, where togavirus-encoded replication apparatus generates copies of the RNA. Using information from the expression cassette encoded RNA, the mammalian ribosome biosynthesizes non-structural viral proteins which, in turn, generate and amplify copies of the RNA. Using this information, the mammalian ribosome also synthesizes the heterologous antigens encoded by the RNA.

Table 4, below, discloses a number of togaviruses and genomes, contemplated for the togavirus-derived expression cassette. Togaviruses, which include alphaviruses and can include flaviviruses, have a single-stranded (+)RNA genome, where the RNA genome contains an open reading frame (ORF) encoding a polyprotein. The togavirus polyprotein contains a protease, which catalyzes cleavage of the polyprotein to generate separate non-structural proteins (nsp). The non-structural proteins of togaviruses include a protease and an RNA polymerase. The RNA polymerase catalyzes replication of the viral genome in the host cell's cytoplasm. In some embodiments, the RNA polymerase catalyzes amplification of the togavirus-derived expression cassette.

The togavirus-derived expression cassette of the invention is described by way of the example of alphaviruses. Alphaviruses are closely related in their genomic organization and include Sindbis virus (SIN), Semliki Forest virus, Venezualan equine encephalitis virus (VEE), Eastern equine encephalitis virus, Western equine encephalitis virus, and Ross River virus (see, e.g., Kuhn, et al. (1996) J. Virol. 70:7900-7909; Powers, et al. (2001) J. Virol. 75:10118-10131; Schlesinger (2001) Exp. Opin. Biol. Ther. 1:177-191; Frolov, et al. (1999) J. Virol. 73:3854-3865). Alphaviruses encode four non-structural proteins (nsp), nsp1, nsp2, nsp3, and nsp4. The DNA of the invention, which can be integrated into the listerial genome, encodes (+)strand RNA, which is cap-independent. The (+)strand RNA is cap-independent because at the early stages of infection, nsp1 (capping enzyme) is not yet expressed. The (+)strand RNA then encodes (−) strand RNA, where the (−) strand RNA encodes both full length genomic (+)strand RNA (which can be capped) and shorter sub-genomic (+)strand RNA, which encodes the structural proteins (or heterologous antigen) (which also can be capped).

After synthesis of the RNA from the alphavirus-derived expression cassette, the RNA can transit from the bacterium to the host cell's cytosol, and the RNA can be used to biosynthesize the polyprotein (P1234), where proteolytic cleavage of P1234 generates the non-structural proteins (nsp), nsp1, nsp2, nsp3, and nsp4. Nsp4 is viral RNA polymerase.

Togavirus genomic structure and polyproteins are described (see, e.g., (Frolov, et al. (1999) J. Virol. 73:3854-3865; Shirako, et al. (2003) J. Virol. 77:2301-2309; Vasiljeva, et al. (2003) J. Biol. Chem. 278:41636-41645; Lampio, et al. (2000) J. Biol. Chem. 275:37853-37859; Vasiljeva, et al. (2001) J. Biol. Chem. 276:30786-30793; Ackermann and Padmanabhan (2001) J. Biol. Chem 276:39926-39937; Wu, et al. (2005) J. Virol. 79:10268-10277); Amberg, et al. (1994) J. Virol. 68:3794-3802).

What is available are nucleic acids encoding alphavirus nsp2 mutants, where the mutation reduces possible cytotoxic effects of the alphavirus-derived expression cassette. These include mutations at amino acids 726 or 779 and at homologous positions in any homologous virus-derived genome (Frolov, et al. (1999) J. Virol. 73:3854-3865).

TABLE 4
Sources of alphavirus- and flavivirus-derived expression cassettes.
Alphaviruses

| | |
|---|---|
| Sindbis virus | Dubensky, et al. (1996) J. Virol. 70: 508-519; Perri, et al. |
| | (2003) J. Virol. 77: 10394-10403; Lindenbach and Rice (1997) |
| | J. Virol. 71: 9608-9617; Perri, et al. (2000) J. Virol. 74: 9802-9807. |
| | See, e.g., GenBank Acc. No. NC_001547. |
| Sindbis virus strain | Smith and Tignor (1980) Arch. Virol. 66: 11-26; Yu, et al. |
| SAAR86 | (1998) J. Biol. Chem. 273: 23524-23533; GenBank Acc. Nos. |
| | AF061686 and AF061207. |
| Semliki Forest | Nordstrom, et al. (2005) J. Gen. Virol. 86: 349-354; Tannis, et |
| | al. (2005) Vaccine 23: 4189-4194; Diatta, et al. (2005) J. Gen. |
| | Virol. 86: 3129-3136; Karlsson and Liljestrom (2004) Methods |
| | Mol. Biol. 246: 543-557; Perri, et al. (2000) J. Virol. 74: 9802-9807; |
| Venezuelan equine | Perri, et al. (2000) J. Virol. 74: 9802-9807; Perri, et al. (2003) |
| encephalitis (VEE) | J. Virol. 77: 10394-10403; Balasuriya, et al. (2000) J. Virol. |
| | 74: 10623-10630; Gehrke, et al. (2005) J. Gen. Virol. 86: 1045-1053; |
| | Cassetti, et al. (2004) Vaccine 22: 520-527; Thompson, |
| | et al. (2006) Proc. Natl. Acad. Sci. USA 103: 3722-3727; |
| Eastern equine | See, e.g., Petrakova, et al. (2005) J. Virol. 79: 7597-7608. |
| encephalitis (EEE) | GenBank Acc. Nos. AY705240; AY722102; AY705241. |
| Western equine | See, e.g., GenBank Acc. No. NC_003908. |
| encephalitis | |
| Ross river virus | See, e.g., Frolov, te al. (1997) J. Virol. 71: 2819-2829; Frolova, |
| | et al. (1997) J. Virol. 71: 248-258; Faragher, et al. (1988) |
| | Virology 163: 509-526; GenBank Acc. No. NC_001544. |
| Sagiyami virus. | See, e.g., Shirako and Yamaguchi (2000) J. Gen. Virol. |
| | 81: 1353-1360. |
| O'Nyong-nyong virus | GenBank Acc. Nos. NC_001512; AF079456; M20303. |
| | Myles, et al. (2006) J. Virol. 49: 4992-4997. |
| Highlands J virus | GenBank Acc. Nos. AF023289; J02206; K00700; AH002349. |
| | Bianchi, et al. (1993) Am. J. Trop. Med. Hyg. 49: 322-328. |

Flaviviruses

| | |
|---|---|
| Yellow fever | Jones, et al. (2005) Virology 331: 247-259; Molenkamp, et al. |
| | (2003) J. Virol. 77: 1644-1648. |
| Yellow fever strain 17D | Lindenbach and Rice (1997) J. Virol. 71: 9608-9617; |
| | Barba-Spaeth, et al. (2005) J. Exp. Med. 202: 1179-1184; |
| | Pugachev, et al. (2005) Curr. Opin. Infect. Dis. 18: 387-394; |
| | Bonaldo, et al. (2005) J. Virol. 79: 8602-8613; Bredenbeek, et |
| | al. (2006) Virology 345: 299-304. See also, e.g., GenBank |
| | Acc. No. X03700. |
| Japanese encephalitis | See, e.g., GenBank Acc. Nos. AB24119; AB24118; |
| | AB196926. |
| St. Louis encephalitis | See, e.g., GenBank Acc. No. NC_007580. |
| Tick-borne encephalitis | Aberle, et al. (2005) J. Virol. 79: 15107-15113; Gehrke, et al. |
| | (2005) J. Gen. Virol. 86: 1045-1053. See, e.g., GenBank Acc. |
| | No. AF069066. |
| Dengue virus | Medlin, et al. (2005) J. Virol. 79: 11053-11061; Alvarez, et al. |
| | (2005) Virology 339: 200-212; Pang, et al. (2001) 1: 28; |
| | Aberle, et al. (2005) J. Virol. 79: 15107-15113; |
| West Nile virus | Aberle, et al. (2005) J. Virol. 79: 15107-15113; Fayzulin, et al. |
| | (2006) Virology April 26 [epub ahead of print]. See, e.g., |
| | GenBank Acc. Nos. DQ411034; DQ411033. |
| Kunjin virus (subtype of | Tannis, et al. (2005) Vaccine 23: 4189-4194; Anraku, et al. |
| West Nile virus) | (2002) J. Virol. 76: 3791-3799; Liu, et al. (2004) J. Virol. |
| | 78: 1225-12235; |

Arterivirus

| | |
|---|---|
| Equine arteritis virus | Pasternak, et al. (2004) J. Virol. 78: 8102-8113. See, e.g., |
| | GenBank Acc. Nos. NC_002532; AY349168. |

Rubivirus

| | |
|---|---|
| Rubella virus | Tzeng, et al. (2005) J. Clin. Microbiol. 43: 879-885; Chen and |
| | Icenogle (2004) J. Virol. 78: 4314-4322; Tzeng and Frey |
| | (2005) Virology 337: 327-334. See, e.g., GenBank Acc. Nos. |
| | AF435866; NC_001545. |

The listerial genome of the invention encompasses a Listeria-compatible transcription start sequence operably linked with a togavirus-derived expression cassette, where transcription in the bacterium produces an RNA, and where the RNA comprises a mammal-compatible transcription start sequence operably linked with at least one open reading frame (ORF), and where the ORF includes at least one nucleic acid encoding a heterologous antigen.

Togavirus-derived expression cassettes, including alphavirus-derived expression cassettes, have been described (See, e.g., Dubensky, et al. (1996) J. Virol. 70:508-519 and U.S. Pat. No. 6,342,372 of Dubensky, et al.). Reagents and methods relating to alphavirus-derived vectors, and to yellow fever virus (a flavivirus)-derived vectors are disclosed. Alphaviruses-based vectors are available (see, e.g., U.S. Pat. No. 5,789,245 issued to Dubensky, et al.; U.S. Pat. No. 5,814,482 issued to Dubensky, et al.; U.S. Pat. No. 5,843,723 issued to Dubensky, et al.; U.S. Pat. No. 6,015,686 issued to Dubensky, et al.; U.S. Pat. No. 6,426,196 issued to Dubensky, et al.; U.S. Pat. No. 6,451,592 issued to Dubensky, et al.; U.S. Pat. No. 6,458,560 issued to Dubensky, et al.; and U.S. Pat. No. 6,465,634 issued to Dubensky, et al.). Yellow fever virus-derived vectors are available (see, e.g., U.S. Pat. No. 6,696,281 issued to Chambers, et al.; U.S. Pat. No. 6,962,708 issued to Chambers, et al., U.S. Pat. No. 5,744,141 issued to Paoletti and Pincus; Bonaldo, et al. (2005) J. Virol. 79:8602-8613; Bredenbeek, et al. (2006) Virology 345:299-304; McAllister, et al. (2000) J. Virol. 74:9197-9295; Tao, et al. (2005) J. Immunol. 201:201-209).

In certain embodiments, the nucleic acid encoding holin and the togavirus-derived expression cassette reside in the same bacterium. But the nucleic acid encoding holin and the togavirus-derived expression cassette need not be supplied by the same bacterium. Rather, they can be provided by two different vectors. Where a first Listeria bacterium provides the togavirus-derived expression cassette, a second Listeria bacterium can provide a nucleic acid encoding holin. In another aspect, the holin can be supplied by a naked nucleic acid vector, adenovirus-derived vector, and so on. What is also provided is a dendric cell (DC) vaccine, where the DC is infected in vitro with the Listeria containing the togavirus-derived cassette and/or the holin (see, e.g., WO 2005/009463).

In other aspects, the invention provides a Listeria bacterium containing an alphavirus-derived expression cassette derived from an alphavirus that tends to stimulate greater interferon response against the alphavirus, such as Sindbis virus, as well as an alphavirus-derived expression cassette derived from an alphavirus that stimulates lesser interferon responses against the alphavirus, such as VEE or yellow fever virus.

### C. Cis-Acting RNA Elements Used in Replication

A cis-acting RNA element for stimulating replication is utilized in some embodiments of the invention, where this element requires nucleotides 5′-prime to the open reading frame encoding non-structural protein-1 (nsp1) and also requires a number of nucleotides within the ORF for nsp1. The cis-acting element overlaps the start codon of nsp1, and encompasses nucleotides both upstream and downstream of this start codon.

In certain embodiments where the invention provides for an IRES, for use in initiating translation of nsp1, the IRES is implanted just upstream of the nsp1 ORF, necessitating disruption of the cis-acting RNA element. The invention thus provides a construct that contains the cis-acting RNA element, as well as the IRES, while avoiding disruption of any part of the cis-acting RNA element. Disruption is avoided by providing an RNA containing the following nucleic acids in the following order, from 5′-prime to 3′-prime direction: First nucleic acid: Complete, intact cis-acting RNA element, where the cis-acting RNA element (in one embodiment) includes at least the part of nsp1 that is necessary for cis-acting RNA element activity; Second nucleic acid: IRES; and Third nucleic acid: Open reading frame for entire nsp1 (Table 5). As is evident from the order of the first, second, and third nucleic acids, part of the nsp1 sequence is duplicated. A potential problem in duplicate regions is the generation of artefacts within the bacterium, where the artefacts are produced by homologous recombination. The invention provides for preventing these artefacts as follows. Regarding the two duplicate regions, the nucleotide sequence of the second duplicate region is changed so that it is no longer homologous to the first duplicate region, while not changing the amino acids that are encoded by the second duplicate region.

TABLE 5
Alphavirus based expression cassette, and components thereof.

| Wild type non- | |
|---|---|
| structural protein | |
| (nspt) sequence. | GenBank Acc. No. NP_062889). |
| IRES sequence, and | Gagctcgtatggacatattgtcgttagaacgcggctacaattaatacat |
| sequences upstream | AaccttatgtatcatacacatacgatttaggggacactatagGGATATA |
| and downstream to the | GTGGTGAGTATCCCCGCCTGTCACGCGGGAGACCGGGGTTCGGTTCCCC |
| IRES sequence. | GACGGGGAGCcaaacagccgaccaattgcactaccatcacaatggagaa |
| (SEQ ID NO: 11) | Gccagtagtaaacgtagacgtagacccccagagtccgtttgtcgtgcaa |
| | Ctgcaaaaaagcttcccgcaatttgaggtagtagcacagcaggtcactc |
| | CaaatgaccatgctaatgccagagcattttcgcatctggcGCATGCATC |
| | TAGGGCGGCCAATTCCGCCCCTCTCCCTCCCCCCCCCCTAACGTTACTG |
| | GCCGAAGCCGCTTGGAATAAGGCCGGTGTGCGTTTGTCTATATGTGATT |
| | TTCCACCATATTGCCGTCTTTTGGCAATGTGAGGGCCCGGAAACCTGGC |
| | CCTGTCTTCTTGACGAGCATTCCTAGGGGTCTTTCCCCTCTCGCCAAAG |
| | GAATGCAAGGTCTGTTGAATGTCGTGAAGGAAGCAGTTCCTCTGGAAGC |
| | TTCTTGAAGACAAACAACGTCTGTAGCGACCCTTTGCAGGCAGCGGAAC |
| | CCCCCACCTGGCGACAGGTGCCTCTGCGGCCAAAAGCCACGTGTATAAG |
| | ATACACCTGCAAAGGCGGCACAACCCCAGTGCCACGTTGTGAGTTGGAT |
| | AGTTGTGGAAAGAGTCAAATGGCTCTCCTCAAGCGTATTCAACAAGGGG |
| | CTGAAGGATGCCCAGAAGGTACCCCATTGTATGGGATCTGATCTGGGGC |
| | CTCGGTGCACATGCTTTACATGTGTTTAGTCGAGGTTAAAAAAACGTCT |
| | AGGCCCCCCGAACCACGGGGACGTGGTTTTCCTTTGAAAAACACGATGA |
| | TAAGCTTatggaaaaaccggtggtcaatgtggatgtcgatccacaaagc |
| | Ccattcgtagtacagcttcagaagtcatttccacagttcgaagtggtcg |
| | Cccagcaagtaaccccgaacgaccacgccaacgcaagagccttcagcca |
| | Cctggcc |
| Sp6 promoter. This | atttaggggacactatag |
| promoter allows | |
| transcription of | |
| downstream material, | |
| including the | |
| DI sequence, a first | |
| nsp1 sequence (nt | |
| corresponding to only | |
| about the first 48 | |
| amino acids of nsp1), | |
| the IRES sequence, | |
| and a second nsp1 | |
| (full-length nsp1 | |
| sequence). | |
| SEQ ID NO: 12) | |
| “DI sequence.” This | GGATATAGTGGTGAGTATCCCCGCCTGTCACGCGGGAGACCGGGG |
| sequence, which | TTCGGTTCCCCGACGGGGAGC |
| resembles tRNA^{ASP}, | |
| enhances stability of | |
| the expressed | |
| message. | |
| (SEQ ID NO: 13) | |
| Sequence that | Caaacagccgaccaattgcactaccatcacaatgga |
| enhances replication. | GaagccagtagtaaacgtagacgTagacccccagag |
| (SEQ ID NO: 14) | Tccgtttgtcgtgcaactgcaaaaaagcttcccgca |
| | Atttgaggtagtagcacagcaggtcactccaaatga |
| | Ccatgctaatgccagagcattttcgcatctggc |

The sequence that enhances replication has two regions: (1) The first
region is upstream of the nsp1-encoding region:
Caaacagccgaccaattgcactaccatcaca; and (2) The second region
is a fragment of the nsp1-encoding region: atg gaG aag cca gta
gta aac gta gac gTa gac ccc cag agT ccg ttt gtc gtg caa ctg caa aaa
agc ttc ccg caA ttt gag gta gta gca cag cag gtc act cca aat gaC cat gct
aat gcc aga gca ttt tcg cat ctg gc (SEQ ID NO: 15).

| | |
|---|---|
| ECVM internal ribosome | GCATGCATCTAGGGCGGCCAATTCCGCCCCTCTCCCTCCCCCCCCCC |
| entry sequence | TAACGTTACTGGCCGAAGCCGCTTGGAATAAGGCCGGTGTGCGTTTG |
| (IRES) sequence. | TCTATATGTGATTTTCCACCATATTGCCGTCTTTTGGCAATGTGAGG |
| (SEQ ID NO: 16) | GCCCGGAAACCTGGCCCTGTCTTCTTGACGAGCATTCCTAGGGGTCT |
| | TTCCCCTCTCGCCAAAGGAATGCAAGGTCTGTTGAATGTCGTGAAGG |
| | AAGCAGTTCCTCTGGAAGCTTCTTGAAGACAAACAACGTCTGTAGCG |
| | ACCCTTTGCAGGCAGCGGAACCCCCCACCTGGCGACAGGTGCCTCTG |
| | CGGCCAAAAGCCACGTGTATAAGATACACCTGCAAAGGCGGCACAAC |
| | CCCAGTGCCACGTTGTGAGTTGGATAGTTGTGGAAAGAGTCAAATGG |
| | CTCTCCTCAAGCGTATTCAACAAGGGGCTGAAGGATGCCCAGAAGGT |
| | ACCCCATTGTATGGGATCTGATCTGGGGCCTCGGTGCACATGCTTTA |
| | CATGTGTTTAGTCGAGGTTAAAAAAACGTCTAGGCCCCCCGAACCAC |
| | GGGGACGTGGTTTTCCTTTGAAAAACACGATGATAAGCTT |
| First 50 codons of the | Atggaaaaaccggtggtcaatgtggatgtcgatccacaa |
| second nsp1 sequence. | Agcccattcgtagtacagcttcagaagtcatttccacag |
| These nucleotides | Ttcgaagtggtcgcccagcaagtaaccccgaacgaccac |
| (nucleotides altered) | gccaacgcaagagccttcagccacctg |
| correspond to the first | |
| nsp1 sequence | |
| (nucleotides not | |
| altered). | |
| To prevent generation | |
| of artifacts in vivo, | |
| resulting from | |
| homologous | |
| recombination, a | |
| number of nucleotides | |
| were changed in the | |
| second nsp1 sequence | |
| (without changing the | |
| encoded amino acids | |
| that were encoded by | |
| the second nsp1 | |
| sequence). | |
| (SEQ ID NO: 17) | |

### D. Internal Ribosome Entry Sites (IRES).

An internal ribosome entry site (IRES), useful for operably linking with a nucleic acid encoding a heterologous antigen, is available for the invention. For example, the invention encompasses a Listeria bacterium containing a togavirus-derived expression cassette, where the expression cassette contains at least one IRES, and where the IRES is operably linked with a nucleic acid encoding a heterologous antigen.

IRES sequences, also called cap-independent translation enhancers (CITE), are stretches of about 400-500 ribonucleotides residing either at the 5′-prime end of mRNA or at internal sites in the mRNA. The IRES sequence is used to initiate translation. In detail, the IRES sequence can mediate entry of a ribosome and initiate translation at an internal site of an mRNA that lacks a cap (see, e.g., Jimenez, et al. (2005) RNA 11:1385-1399; Lytle, et al. (2001) J. Virol. 75:7629-7636; Boni, et al. (2005) J. Biol. Chem. 280:17737-17748; Belsham and Sonenberg (1996) Microbiol. Revs. 60:499-551; Makrides (1999) Protein Expression and Purification 17:183-202; Borman, et al. (1997) Nucl. Acids Res. 25:925-932; Mountford and Smith (1995) Trends Genet. 11:179-184). IRES sequences can be useful in the following situation. The eukaryotic translation machinery sometimes cannot use the second ORF of a bicistronic message. However, where an IRES resides upstream (5′-prime) to the second open reading frame, the eukaryotic translation machinery readily uses the second ORF for polypeptide synthesis.

IRES sequences available for the reagents and methods of the invention include, but are not limited to, IRES sequences from hepatoviruses (e.g., hepatitis A virus; hepatitis C virus), cardioviruses (e.g., encephalomyocarditis virus; mengovirus; Theiler's murine encephalomyelitis virus; echovirus 22), aphthaoviruses (e.g., foot and mouth disease virus (FMDV)), rhinoviruses, and enteroviruses (e.g., polioviruses; coxsackie A21 virus; enterovirus 70; coxsackie B virus; coxsackie A9 viruses; coxsackie A16 virus; echoviruses, and bovine enterovirus). IRES sequences occur in pestiviruses and GB virus B, picornaviruses, simian immunodeficiency virus (SIV), retro-elements such as VL-30, and retroviruses (e.g., Friend murine leukemia virus; Moloney murine leukemia virus (MMLV); human T-cell leukemia virus; reticuloendotheliosis virus type A). A number of IRES sequences have also been identified in mRNAs encoding mammalian proteins (“cellular IRES”) (see, e.g., Chappell and Mauro (2003) J. Biol. Chem. 278:33793-33800; Bornes, et al. (2004) J. Biol. Chem. 279:18717-18726; Ali, et al. (2000) J. Biol. Chem. 275:27531-27540; Komar and Hatzoglou (2005) J. Biol. Chem. 280:23425-23428; Jackson and Kaminski (1995) RNA 1:985-1000; Fernandez, et al. (2001) J. Biol. Chem. 276:12285-12291; Ohlmann, et al. (2000) J. Biol. Chem. 275:11899-11906; Sachs (2000) Cell 101:243-245; Stoneley and Willis (2004) Oncogene 23:3200-3207). IRES elements from one or more of HRV (110 to 640, numbered from 5′-end of viral genome), FMDV (-445 to 1, numbered from initiation codon), HAV (225 to 746, numbered from 5′-end of genome), HCV (40 to 380, numbered from 5′-end of genome), GBV-B (61 to 460), GBV-C (60 to 690), CSFV (65-376, numbered from 5′-end of genome), and HHV8 (−225 to 1, numbered from initiation codon), are available for use in the present invention (see, e.g., Beales, et al. (2003) J. Virol. 77:6574-6579).

Table 6 discloses a number of IRES sequences, available for use in the invention, e.g., where the IRES can be integrated near or at the 5′-prime end of a togavirus-derived expression cassette, at an internal position in the togavirus-derived expression cassette, and where the IRES is operably linked with a nucleic acid encoding an open reading frame (ORF).

TABLE 6
Internal ribosome entry site (IRES) sequences.

| | |
|---|---|
| Source | Sequence |

| | |
|---|---|
| Encephalomyocarditis | Tcccccccccctaacgttactggccgaagccgcttggaataaggccg |
| virus | Gtgtgcgtttgtctatatgttattttccaccatattgccgtcttttg |
| strain HB1 IRES | Gcaatgtgagggcccggaaacctggccctgtcttcttgacgagcatt |
| (nt 167-745 | Cctaggggtctttcccctctcgccaaaggaatgcaaggtctgttgaa |
| of GenBank Acc. No. | Tgtcgtgaaggaagcagttcctctggaagcttcttgaagacaaacaa |
| DQ464063). See | Cgtctgtagcgaccctttgcaggcagcggaaccccccacctggcgac |
| also, nt 302-880 of | Aggtgcctctgcggccaaaagccacgtgtataagatacacctgcaaa |
| GenBank Acc. No. | Ggcggcacaaccccagtgccacgttgtgagttggatagttgtggaaa |
| X74312; and | Gagtcaaatggctctcctcaagcgtattcaacaaggggctgaaggat |
| Kaminski and | Gcccagaaggtaccccattgtatgggatctgatctggggcctcggtg |
| Jackson (1998) RNA | Cacatgctttacatgtgtttagtcgaggttaaaaaacgtctaggccc |
| 4: 626-638). | Ccctaaccacggggacgtggttttcctttgaaaaacacgatgataat |
| (SEQ ID NO: 18) | Atggccacaaccatggaacaagagac |
| Hepatitis C | Agaccacaacggtttccctctagcgggatcaattccgcccctctccc |
| Virus IRES | Tcccccccccctaacgttactggccgaagccgcttggaataaggccg |
| (GenBank Acc. No. | Gtgtgcgtttgtctatatgttattttccaccatattgccgtcttttg |
| AJ242653) (nt | Gcaatgtgagggcccggaaacctggccctgtcttcttgacgagcatt |
| 1202-1812) | Cctaggggtctttcccctctcgccaaaggaatgcaaggtctgttgaa |
| (SEQ ID NO: 19) | Tgtcgtgaaggaagcagttcctctggaagcttcttgaagacaaacaa |
| | Cgtctgtagcgaccctttgcaggcagcggaaccccccacctggcgac |
| | Aggtgcctctgcggccaaaagccacgtgtataagatacacctgcaaa |
| | Ggcggcacaaccccagtgccacgttgtgagttggatagttgtggaaa |
| | Gagtcaaatggctctcctcaagcgtattcaacaaggggctgaaggat |
| | Gcccagaaggtaccccattgtatgggatctgatctggggcctcggtg |
| | Cacatgctttacatgtgtttagtcgaggttaaaaaacgtctaggccc |
| | cccgaaccacggggacgtggttttcctttgaaaaacacgataatacc |
| Foot and mouth | Ggtttccacaactgataaaactcgtgcaacttgaaactccgcctggt |
| disease virus O IRES | Ctttccaggtctagaggggttacactttgtactgtgctcgactccac |
| GenBank Acc. No. | Gcccggtccactggcgggtgttagtagcagcactgttgtttcgtagc |
| NC004004) | Ggagcatggtggccgtgggaactcctccttggtgacaagggcccacg |
| (nt 600-1058). | Gggccgaaagccacgtccagacggacccaccatgtgtgcaaccccag |
| (SEQ ID NO: 20) | Cacggcaacttttactgcgaacaccaccttaaggtgacactggtact |
| | Ggtactcggtcactggtgacaggctaaggatgcccttcaggtacccc |
| | Gaggtaacacgggacactcgggatctgagaaggggattgggacttct |
| | Ttaaaagtgcccagtttaaaaagcttctacgcctgaataggcgaccg |
| | gaggccggcgcctttccattacccacta ctaaatcc |
| Kunjin virus IRES. | Khromykh and Westaway (1997) J. Virol. 71: 1497-1505. (Kunjin is in |
| | Flaviviridae family.) |
| Encephalomyocarditis | Hoffman and Palmenberg (1995) J. Virol. 69: 4399-4406; Pugachev, et al. |
| virus (EMCV) IRES. | (2000) J. Virol. 74: 10811-10815. (Rubella virus is in Togaviridae |
| | family.) |
| GB virus B (GBV-B) | Rijnbrand, et al. (2000) J. Virol. 74: 773-783. (GB virus is in |
| IRES. | Flaviviridae family). GB virus B IRES has no specific requirement |
| | for polyprotein sequences (Rijnbrand, et al., supra). |
| Foot-and-mouth | Meyer, et al. (1995) J. Virol. 69: 2819-2824. |
| disease virus IRES. | |
| Echovirus IRES. | Bradrick, et al. (2001) J. Virol. 75: 6472-6481. Echovirus IRES |
| | sequences is functional with engineered into coxsackievirus (Bradrick, et |
| | al., supra). |
| Hepatitis C virus | Jubin, et al. (2000) J. Virol. 74: 10430-10437. |
| IRES. | |
| Swine fever virus | Fletcher and Jackson (2002) J. Virol. 76: 5024-5033. |
| (CSFV) IRES. | |
| Lymphoid enhancer | Jimenez, et al. (2005) RNA 11: 1385-1399. |
| factor-1 IRES. | |
| c-myc IRES. | Nanbru, et al. (1997) J. Biol. Chem. 272: 32061-32066. |
| FGF-2 IRES. | Nanbru, et al. (1997) J. Biol. Chem. 272: 32061-32066. |

### E. Stabilizing Nucleic Acids Useful in Virus-Derived Expression Cassettes (e,g., Togavirus-Derived Expression Cassettes).

Nucleic acids that maintain or enhance stability of the RNA expressed from a virus-derived expression cassette (including, but not limited to, togavirus-derived expression cassette), or enhance stability of message amplified from the RNA, are available. Stabilizing nucleic acids includes those residing at or near the 5′-prime end of an expressed RNA. Suitable stabilizing nucleic acids include, but are not limited to, tRNA-like structures and structures derived from tRNA (see, e.g., Agapov, et al. (1998) Proc. Natl. Acad. Sci. USA 95:12989-12994; Monroe and Schlesinger (1983) Proc. Natl. Acad. Sci. USA 80:3279-3283).

Table 7 discloses a number of useful stabilizing structures. The effect of the stabilizing nucleic acid can be measured, or inferred from, the intracellular concentration of the RNA, size range of the RNA, expression of a polypeptide from the RNA, immune response to a polypeptide from the RNA, and the like.

TABLE 7

Nucleic acids encoding a stabilizing structure, useful at or
near the 5′-prime end of a virus-derived expression cassette
(e,g., togavirus-derived expression cassette).

| | |
|---|---|
| Sequence resembling | Atatagtggtgagtatccccgcctgtcacgcgggagac |
| tRNA^{Asp} identified in an | cggggttcggttccccgacggggagcca |
| alphaviral Defective | |
| Interfering (DI) particle | |
| (Monroe and | |
| Schlesinger (1983) | |
| Proc. Natl. Acad. Sci. | |
| USA 80: 3279-3283). | |
| (SEQ ID NO: 21) | |
| tRNA^{Asp} (Sekiya, et al. | tcctcgttagtatagtggtgagtatccccgcctgtcac |
| (1981) Nucleic Acids | gcgggagaccggggttcgattccccgacggggag |
| Res. 9: 2239-2250; | |
| GenBank Acc. No. | |
| V01272) | |
| (SEQ ID NO: 22) | |

Available stabilizing nucleic acids include sequences that bind an RNA-binding protein, for example, La protein and iron response element (IRE) (see, e.g., Heise, et al. (2001) J. Virol. 75:6874-6883). Another available stabilizing structure is the 5′-UTR of a stable mammalian mRNA, such as that for β-globin (Makrides (1999) Protein Expression Purification 17:183-202; Hedley, et al. (1998) Hum. Gene Ther. 9:325-332; Strong, et al. (1997) Gene Ther. 4:624-627). Increased stability can also be provided, in the invention, by including, in the construct, a nucleic acid encoding a non-togavirus capping enzyme (see, e.g., Ahola and Kaariainen (1995) Proc. Natl. Acad. Sci. USA 92:507-511; Vasiljeva, et al. (2000) J. Biol. Chem. 275:17281-17287). Stabilizing nucleic acids also encompass using a togavirus-derived expression cassette with potential RNase cleavage sites removed. Other stabilizing nucleic acids are disclosed (see, e.g., Arnold, et al. (1998) RNA 4:319-330; Bouvet, et al. (1992) Nature 360:488-491; Heck, et al. (1996) Mol. Microbiol. 20:1165-1178; Matsunaga, et al. (1996) RNA 2:1228-1240).

Codon optimization can be applied to the nucleic acid encoding the togavirus-derived expression cassette. Codon optimization can be applied to the proteins of viral origin, as well as to the heterologous antigen (e.g., tumor antigens; hepatitis virus antigens) proteins. Although a variety of viruses infect human cells, and although most tumor antigens are human antigens, what is contemplated, in some embodiments, is improved polypeptide biosynthesis accomplished by codon optimization for expression in human cells. Guidance in codon optimization, for example, using a human consensus codon usage table, for expresson in human cells is available (see, e.g., Ivory and Chadee (2004) Genetic Vaccines and Therapy 2:17-25; Makrides (1999) Protein Expression Purification 17:183-202; Ko, et al. (2005) Infection Immunity 73:5666-5674).

### IV. Listeria

In some embodiments, the Listeria belong to the species Listeria monocytogenes. In some alternative embodiments the bacteria are members of the Listeria ivanovii, Listeria seeligeri, Listeria innocua, L. Welshimeri, or L. grayi species.

In some embodiments, the Listeria are non-naturally occurring. In some embodiments, the Listeria are attenuated. In some embodiments, the Listeria are viable. In some embodiments, the Listeria are mutant Listeria, recombinant Listeria, or otherwise modified. In some embodiments, the Listeria are attenuated. In some embodiments, the Listeria are metabolically active. In certain embodiments, the Listeria are not infected with bacteriophage. The invention further provides Listeria that are recombinant. In addition, the Listeria may be isolated and/or substantially purified.

In some embodiments, the attenuated Listeria is attenuated in one or more of growth, cell to cell spread, binding to or entry into a host cell, replication, or DNA repair. In some embodiments, the Listeria is attenuated by one or more of an actA mutation, an inlB mutation, a uvrA mutation, a uvrB mutation, a uvrC mutation, a nucleic acid targeting compound, or a uvrAB mutation and a nucleic acid targeting compound. In some embodiments, the attenuated Listeria is attenuated in cell to cell spread and/or entry into nonphagocytic cells. In some embodiments, the Listeria is attenuated by one or more of an actA mutation or an actA mutation and an inlB mutation. In some embodiments, the Listeria is ΔactA or ΔactAΔinlB.

In some embodiments, the attenuated Listeria is attenuated for cell-to-cell spread. In some embodiments, the Listeria attenuated for cell-to-cell spread are defective with respect to ActA (e.g., relative to the non-modified or wild-type Listeria). In some embodiments, the Listeria comprises an attenuating mutation in the actA gene. In some embodiments, the Listeria comprises a full or partial deletion in the actA gene.

In some embodiments, the capacity of the attenuated Listeria bacterium for cell-to-cell spread is reduced by at least about 10%, at least about 25%, at least about 50%, at least about 75%, or at least about 90%, relative to Listeria without the attenuating mutation (e.g., wild type Listeria). In some embodiments, the capacity of the attenuated Listeria bacterium for cell-to-cell spread is reduced by at least about 25% relative to Listeria without the attenuating mutation. In some embodiments, the capacity of the attenuated Listeria bacterium attenuated for cell-to-cell spread is reduced by at least about 50% relative to the Listeria without the attenuating mutation.

In vitro assays for determining whether a Listeria bacterium is attenuated for cell-to-cell spread are known to those of ordinary skill in the art. For example, the diameter of plaques formed over a time course after infection of selected cultured cell monolayers can be measured. Plaque assays within L2 cell monolayers can be performed as described previously in Sun, A., A. Camilli, and D. A. Portnoy. 1990, Isolation of Listeria monocytogenes small-plaque mutants defective for intracellular growth and cell-to-cell spread. Infect. Immun. 58:3770-3778, with modifications to the methods of measurement, as described by in Skoble, J., D. A. Portnoy, and M. D. Welch. 2000, Three regions within ActA promote Arp2/3 complex-mediated actin nucleation and Listeria monocytogenes motility. J Cell Biol. 150:527-538. In brief, L2 cells are grown to confluency in six-well tissue culture dishes and then infected with bacteria for 1 h. Following infection, the cells are overlayed with media warmed to 40° C. that is comprised of DME containing 0.8% agarose, Fetal Bovine Serum (e.g., 2%), and a desired concentration of Gentamicin. The concentration of Gentamicin in the media dramatically affects plaque size, and is a measure of the ability of a selected Listeria strain to effect cell-to-cell spread (Glomski, I J., M. M. Gedde, A. W. Tsang, J. A. Swanson, and D. A. Portnoy. 2002. 1 Cell Biol. 156:1029-1038). For example, in some embodiments at 3 days following infection of the monolayer the plaque size of Listeria strains having a phenotype of defective cell-to-cell spread is reduced by at least 50% as compared to wild-type Listeria, when overlayed with media containing Gentamicin at a concentration of 50 μg/ml. On the other hand, the plaque size between Listeria strains having a phenotype of defective cell-to-cell spread and wild-type Listeria is similar when infected monolayers are overlayed with media+agarose containing only 5 μg/ml gentamicin. Thus, the relative ability of a selected strain to effect cell-to-cell spread in an infected cell monolayer relative to wild-type Listeria can be determined by varying the concentration of gentamicin in the media containing agarose. Optionally, visualization and measurement of plaque diameter can be facilitated by the addition of media containing Neutral Red (GIBCO BRL; 1:250 dilution in DME+agarose media) to the overlay at 48 h. post infection. Additionally, the plaque assay can be performed in monolayers derived from other primary cells or continuous cells. For example HepG2 cells, a hepatocyte-derived cell line, or primary human hepatocytes can be used to evaluate the ability of selected Listeria mutants to effect cell-to-cell spread, as compared to wild-type Listeria. In some embodiments, Listeria comprising mutations or other modifications that attenuate the Listeria for cell-to-cell spread produce “pinpoint” plaques at high concentrations of gentamicin (about 50 μg/ml).

In some embodiments, the Listeria is attenuated for entry into non-phagocytic cells (relative or the non-mutant or wildtype Listeria). In some embodiments, the Listeria is defective with respect to one or more internalins (or equivalents). In some embodiments, the Listeria is defective with respect to internalin A. In some embodiments, the Listeria is defective with respect to internalin B. In some embodiments, the Listeria comprise a mutation in inlA. In some embodiments, the Listeria comprise a mutation in inlB. In some embodiments, the Listeria comprise a mutation in both actA and inlB. In some embodiments, the Listeria is deleted in functional ActA and internalinB. In some embodiments, the attenuated Listeria bacterium is an ΔactAΔinlB double deletion mutant. In some embodiments, the Listeria bacterium is defective with respect to both ActA and internalin B.

In some embodiments, the capacity of the attenuated Listeria bacterium for entry into non-phagocytic cells is reduced by at least about 10%, at least about 25%, at least about 50%, at least about 75%, or at least about 90%, relative to Listeria without the attenuating mutation (e.g., the wild type bacterium). In some embodiments, the capacity of the attenuated Listeria bacterium for entry into non-phagocytic cells is reduced by at least about 25% relative to Listeria without the attenuating mutation. In some embodiments, the capacity of the attenuated bacterium for entry into non-phagocytic cells is reduced by at least about 50% relative to Listeria without the attenuating mutation. In some embodiments, the capacity of the attenuated Listeria bacterium for entry into non-phagocytic cells is reduced by at least about 75% relative to Listeria without the attenuating mutation.

In some embodiments, the attenuated Listeria is not attenuated for entry into more than one type of non-phagocytic cell. For instance, the attenuated strain may be attenuated for entry into hepatocytes, but not attenuated for entry into epithelial cells. As another example, the attenuated strain may be attenuated for entry into epithelial cells, but not hepatocytes. It is also understood that attenuation for entry into a non-phagocytic cell of a particular modified Listeria is a result of mutating a designated gene, for example a deletion mutation, encoding an invasin protein which interacts with a particular cellular receptor, and as a result facilitates infection of a non-phagocytic cell. For example, Listeria ΔinlB mutant strains are attenuated for entry into non-phagocytic cells expressing the hepatocyte growth factor receptor (c-met), including hepatocyte cell lines (e.g., HepG2), and primary human hepatocytes.

In some embodiments, even though the Listeria is attenuated for entry into non-phagocytic cells, the Listeria is still capable of uptake by phagocytic cells, such as at least dendritic cells and/or macrophages. In one embodiment the ability of the attenuated Listeria to enter phagocytic cells is not diminished by the modification made to the strain, such as the mutation of an invasin (i.e. approximately 95% or more of the measured ability of the strain to be taken up by phagocytic cells is maintained post-modification). In other embodiments, the ability of the attenuated Listeria to enter phagocytic cells is diminished by no more than about 10%, no more than about 25%, no more than about 50%, or no more than about 75%.

In some embodiments of the invention, the amount of attenuation in the ability of the Listeria to enter non-phagocytic cells ranges from a two-fold reduction to much greater levels of attenuation. In some embodiments, the attenuation in the ability of the Listeria to enter non-phagocytic cells is at least about 0.3 log, about 1 log, about 2 log, about 3 log, about 4 log, about 5 log, or at least about 6 log. In some embodiments, the attenuation is in the range of about 0.3 to >8 log, about 2 to >8 log, about 4 to >8 log, about 6 to >8 log, about 0.3-8 log, also about 0.3-7 log, also about 0.3-6 log, also about 0.3-5 log, also about 0.3-4 log, also about 0.3-3 log, also about 0.3-2 log, also about 0.3-1 log. In some embodiments, the attenuation is in the range of about 1 to >8 log, 1-7 log, 1-6 log, also about 2-6 log, also about 2-5 log, also about 3-5 log.

In vitro assays for determining whether or not a Listeria bacterium is attenuated for entry into non-phagocytic cells are known to those of ordinary skill in the art. For instance, both Dramsi et al., Molecular Microbiology 16:251-261 (1995) and Gaillard et al., Cell 65:1127-1141 (1991) describe assays for screening the ability of mutant L. monocytogenes strains to enter certain cell lines. For instance, to determine whether a Listeria bacterium with a particular modification is attenuated for entry into a particular type of non-phagocytic cells, the ability of the attenuated Listeria bacterium to enter a particular type of non-phagocytic cell is determined and compared to the ability of the identical Listeria bacterium without the modification to enter non-phagocytic cells. Likewise, to determine whether a Listeria strain with a particular mutation is attenuated for entry into a particular type of non-phagocytic cells, the ability of the mutant Listeria strain to enter a particular type of non-phagocytic cell is determined and compared to the ability of the Listeria strain without the mutation to enter non-phagocytic cells. For instance, the ability of a modified Listeria bacterium to infect non-phagocytic cells, such as hepatocytes, can be compared to the ability of non-modified Listeria or wild type Listeria to infect phagocytic cells. In such an assay, the modified and non-modified Listeria is typically added to the non-phagocytic cells in vitro for a limited period of time (for instance, an hour), the cells are then washed with a gentamicin-containing solution to kill any extracellular bacteria, the cells are lysed and then plated to assess titer. Examples of such an assay are found in U.S. Patent Publication No. 2004/0228877. In addition, confirmation that the strain is defective with respect to internalin B may also be obtained through comparison of the phenotype of the strain with the previously reported phenotypes for internalin B mutants.

A Listeria monocytogenes ΔactAΔinlB strain was deposited with the American Type Culture Collection (ATCC), 10801 University Blvd., Manassas, Va. 20110-2209, United States of America (P.O. Box 1549, Manassas, Va., 20108, United States of America), on Oct. 3, 2003, under the provisions of the Budapest Treaty on the International Recognition of the Deposit of Microorganisms for the Purposes of Patent Procedure, and designated with accession number PTA-5562. Another Listeria monocytogenes strain, an ΔactA ΔuvrAB strain, was also deposited with the ATCC on Oct. 3, 2003, under the provisions of the Budapest Treaty on the International Recognition of the Deposit of Microorganisms for the Purposes of Patent Procedure, and designated with accession number PTA-5563.

In some embodiments, Listeria is attenuated for nucleic acid repair (e.g., relative to wildtype). For instance, in some embodiments, the Listeria is defective with respect to at least one DNA repair enzyme (e.g., Listeria monocytogenes uvrAB mutants). In some embodiments, the Listeria is defective with respect to PhrB, UvrA, UvrB, UvrC, UvrD, and/or RecA. In some embodiments, the bacteria are defective with respect to UvrA, UvrB, and/or UvrC. In some embodiments, the bacteria comprise attenuating mutations in phrB, uvrA, uvrB, uvrC, uvrD, and/or recA genes. In some embodiments, the bacteria comprise one or more mutations in the uvrA, uvrB, and/or uvrC genes. In some embodiments, the bacteria are functionally deleted in UvrA, UvrB, and/or UvrC. In some embodiments, the bacteria are deleted in functional UvrA and UvrB. In some embodiments, the bacteria are uvrAB deletion mutants. In some embodiments, the bacteria are ΔuvrABΔactA mutants. In some embodiments, the nucleic acid of the bacteia which are attenuated for nucleic acid repair and/or are defective with respect to at least one DNA repair enzyme are modified by reaction with a nucleic acid targeting compound. Nucleic acid repair mutants, such as ΔuvrAB Listeria monocytogenes mutants, and methods of making the mutants, are described in detail in U.S. Patent Publication No. 2004/0197343, which is incorporated by reference herein in its entirety (see, e.g., Example 7 of U.S. 2004/0197343).

In some embodiments, the capacity of the attenuated Listeria bacterium for nucleic acid repair is reduced by at least about 10%, at least about 25%, at least about 50%, at least about 75%, or at least about 90%, relative to a Listeria bacterium without the attenuating mutation (e.g., the wild type bacterium). In some embodiments, the capacity of the attenuated Listeria bacterium for nucleic acid repair is reduced by at least about 25% relative to a Listeria bacterium without the attenuating mutation. In some embodiments, the capacity of the attenuated Listeria bacterium attenuated for nucleic acid repair is reduced by at least about 50% relative a Listeria bacterium without the attenuating mutation.

Confirmation that a particular mutation is present in a bacterial strain can be obtained through a variety of methods known to those of ordinary skill in the art. For instance, the relevant portion of the strain's genome can be cloned and sequenced. Alternatively, specific mutations can be identified via PCR using paired primers that code for regions adjacent to a deletion or other mutation. Southern blots can also be used to detect changes in the bacterial genome. Also, one can analyze whether a particular protein is expressed by the strain using techniques standard to the art such as Western blotting. Confirmation that the strain contains a mutation in the desired gene may also be obtained through comparison of the phenotype of the strain with a previously reported phenotype. For example, the presence of a nucleotide excision repair mutation such as deletion of uvrAB can be assessed using an assay which tests the ability of the bacteria to repair its nucleic acid using the nucleotide excision repair (NER) machinery and comparing that ability against wild-type bacteria. Such functional assays are known in the art. For instance, cyclobutane dimer excision or the excision of UV-induced (6-4) products can be measured to determine a deficiency in an NER enzyme in the mutant (see, e.g., Franklin et al., Proc. Natl. Acad. Sci. USA, 81: 3821-3824 (1984)). Alternatively, survival measurements can be made to assess a deficiency in nucleic acid repair. For instance, the Listeria can be subjected to psoralen/UVA treatment and then assessed for their ability to proliferate and/or survive in comparison to wild-type.

The invention supplies a number of Listeria strains for making or engineering an attenuated Listeria of the present invention (Table 8). The Listeria of the present invention are not to be limited by the strains disclosed in this table.

TABLE 8
Exemplary strains of Listeria for use as parental
strains in the present invention.

| | |
|---|---|
| L. monocytogenes 10403S wild type. | Bishop and Hinrichs (1987) J. Immunol. |
| | 139: 2005-2009; Lauer, et al. (2002) J. |
| | Bact. 184: 4177-4186. |
| L. monocytogenes DP-L4056 (phage cured). | Lauer, et al. (2002) J. Bact. 184: 4177-4186. |
| The prophage-cured 10403S strain is | |
| designated DP-L4056. | |
| L. monocytogenes DP-L4027, which is | Lauer, et al. (2002) J. Bact. 184: 4177-4186; |
| DP-L2161, phage cured, deleted in hly gene. | Jones and Portnoy (1994) Infect. |
| | Immunity 65: 5608-5613. |
| L. monocytogenes DP-L4029, which is DP- | Lauer, et al. (2002) J. Bact. 184: 4177-4186; |
| L3078, phage cured, deleted in actA. | Skoble, et al. (2000) J. Cell Biol. |
| | 150: 527-538. |
| L. monocytogenes DP-L4042 (delta PEST) | Brockstedt, et al. (2004) Proc. Natl. Acad. |
| | Sci. USA 101: 13832-13837; supporting |
| | information. |
| L. monocytogenes DP-L4097 (LLO-S44A). | Brockstedt, et al. (2004) Proc. Natl. Acad. |
| | Sci. USA 101: 13832-13837; supporting |
| | information. |
| L. monocytogenes DP-L4364 (delta lplA; | Brockstedt, et al. (2004) Proc. Natl. Acad. |
| lipoate protein ligase). | Sci. USA 101: 13832-13837; supporting |
| | information. |
| L. monocytogenes DP-L4405 (delta inlA). | Brockstedt, et al. (2004) Proc. Natl. Acad. |
| | Sci. USA 101: 13832-13837; supporting |
| | information. |
| L. monocytogenes DP-L4406 (delta inlB). | Brockstedt, et al. (2004) Proc. Natl. Acad. |
| | Sci. USA 101: 13832-13837; supporting |
| | information. |
| L. monocytogenes CS-L0001 (delta actA- | Brockstedt, et al. (2004) Proc. Natl. Acad. |
| delta inlB). | Sci. USA 101: 13832-13837; supporting |
| | information. |
| L. monocytogenes CS-L0002 (delta actA- | Brockstedt, et al. (2004) Proc. Natl. Acad. |
| delta lplA). | Sci. USA 101: 13832-13837; supporting |
| | information. |
| L. monocytogenes CS-L0003 (L461T-delta | Brockstedt, et al. (2004) Proc. Natl. Acad. |
| lplA). | Sci. USA 101: 13832-13837; supporting |
| | information. |
| L. monocytogenes DP-L4038 (delta actA- | Brockstedt, et al. (2004) Proc. Natl. Acad. |
| LLO L461T). | Sci. USA 101: 13832-13837; supporting |
| | information. |
| L. monocytogenes DP-L4384 (S44A-LLO | Brockstedt, et al. (2004) Proc. Natl. Acad. |
| L461T). | Sci. USA 101: 13832-13837; supporting |
| | information. |
| L. monocytogenes. Mutation in lipoate | O'Riordan, et al. (2003) Science 302: 462-464. |
| protein ligase (LplA1). | |
| L. monocytogenes DP-L4017 (10403S with | U.S. Provisional Pat. Appl. Ser. No. |
| LLO L461T point mutation in hemolysin | 60/490,089 filed Jul. 24, 2003. |
| gene). | |
| L. monocytogenes EGD. | GenBank Acc. No. AL591824. |
| L. monocytogenes EGD-e. | GenBank Acc. No. NC_003210. ATCC |
| | Acc. No. BAA-679. |
| L. monocytogenes strain EGD, complete | GenBank Acc. No. AL591975 |
| genome, segment 3/12 | |
| L. monocytogenes. | ATCC Nos. 13932; 15313; 19111-19120; |
| | 43248-43251; 51772-51782. |
| L. monocytogenes DP-L4029 deleted | U.S. Provisional Pat. Appl. Ser. No. |
| in uvrAB. | 60/541,515 filed Feb. 2, 2004; U.S. |
| | Provisional Pat. Appl. Ser. No. 60/490,080 |
| | filed Jul. 24, 2003. |
| L. monocytogenes DP-L4029 deleted | U.S. Provisional Pat. Appl. Ser. No. |
| in uvrAB treated with a psoralen. | 60/541,515 filed Feb. 2, 2004. |
| L. monocytogenes actA⁻/inlB⁻ double mutant. | Deposited with ATCC on Oct. 3, 2003. |
| | Acc. No. PTA-5562. |
| L. monocytogenes lplA mutant or hly | U.S. Pat. Applic. No. 20040013690 of |
| mutant. | Portnoy, et al. |
| L. monocytogenes DAL/DAT double | U.S. Pat. Applic. No. 20050048081 of |
| mutant. | Frankel and Portnoy. |
| L. monocytogenes str. 4b F2365. | GenBank Acc. No. NC_002973. |
| Listeria ivanovii | ATCC No. 49954 |
| Listeria innocua Clip11262. | GenBank Acc. No. NC_003212; |
| | AL592022. |
| Listeria innocua, a naturally occurring | Johnson, et al. (2004) Appl. Environ. |
| hemolytic strain containing the | Microbiol. 70: 4256-4266. |
| PrfA-regulated virulence gene cluster. | |
| Listeria seeligeri. | Howard, et al. (1992) Appl. Eviron. |
| | Microbiol. 58: 709-712. |
| Listeria innocua with L. monocytogenes | Johnson, et al. (2004) Appl. Environ. |
| pathogenicity island genes. | Microbiol. 70: 4256-4266. |
| Listeria innocua with L. monocytogenes | See, e.g., Lingnau, et al. (1995) Infection |
| internalin A gene, e.g., as a plasmid or as a | Immunity 63: 3896-3903; Gaillard, et al. |
| genomic nucleic acid. | (1991) Cell 65: 1127-1141). |

The present invention encompasses reagents and methods that comprise the above listerial strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); daaA (dat; D-amino acid aminotransferase); plcA; plcB; actA; or any nucleic acid that mediates growth, spread, breakdown of a single walled vesicle, breakdown of a double walled vesicle, binding to a host cell, uptake by a host cell. The present invention is not to be limited by the particular strains disclosed above.

In some embodiments, the attenuation of Listeria can be measured in terms of biological effects of the Listeria on a host. The pathogenicity of a strain can be assessed by measurement of the LD₅₀ in mice or other vertebrates. The LD₅₀ is the amount, or dosage, of Listeria injected into vertebrates necessary to cause death in 50% of the vertebrates. The LD₅₀ values can be compared for bacteria having a particular modification (e.g., mutation) versus the bacteria without the particular modification as a measure of the level of attenuation. For example, if the bacterial strain without a particular mutation has an LD₅₀ of 10³ bacteria and the bacterial strain having the particular mutation has an LD₅₀ of 10⁵ bacteria, the strain has been attenuated so that is LD₅₀ is increased 100-fold or by 2 log.

In some embodiments, the attenuated Listeria has an LD₅₀ that is at least about 5 times higher, at least about 10 times higher, at least about 100 times higher, at least about 1000 times higher, or at least about 1×10⁴ higher than the LD₅₀ of parental or wildtype Listeria.

As a further example, the degree of attenuation may also be measured qualitatively by other biological effects, such as the extent of tissue pathology or serum liver enzyme levels. Alanine aminotransferase (ALT), aspartate aminotransferase (AST), albumin and bilirubin levels in the serum are determined at a clinical laboratory for mice injected with Listeria (or other bacteria). Comparisons of these effects in mice or other vertebrates can be made for Listeria with and without particular modifications/mutations as a way to assess the attenuation of the Listeria. Attenuation of the Listeria may also be measured by tissue pathology. The amount of Listeria that can be recovered from various tissues of an infected vertebrate, such as the liver, spleen and nervous system, can also be used as a measure of the level of attenuation by comparing these values in vertebrates injected with mutant versus non-mutant Listeria. For instance, the amount of Listeria that can be recovered from infected tissues such as liver or spleen as a function of time can be used as a measure of attenuation by comparing these values in mice injected with mutant vs. non-mutant Listeria.

Accordingly, the attenuation of the Listeria can be measured in terms of bacterial load in particular selected organs in mice known to be targets by wild-type Listeria. For example, the attenuation of the Listeria can be measured by enumerating the colonies (Colony Forming Units; CFU or cfu) arising from plating dilutions of liver or spleen homogenates (homogenized in H₂0+0.2% NP40) on BHI agar media. The liver or spleen cfu can be measured, for example, over a time course following administration of the modified Listeria via any number of routes, including intravenous, intraperitoneal, intramuscular, and subcutaneous. Additionally, the Listeria can be measured and compared to a drug-resistant, wild type Listeria (or any other selected Listeria strain) in the liver and spleen (or any other selected organ) over a time course following administration by the competitive index assay, as described.

Methods of producing mutant Listeria are well known in the art. Bacterial mutations can be achieved through traditional mutagenic methods, such as mutagenic chemicals or radiation followed by selection of mutants. Bacterial mutations can also be achieved by one of skill in the art through recombinant DNA technology. For instance, the method of allelic exchange using the pKSV7 vector described in Camilli et al., Molecular Micro. 8:143-157 (1993) is suitable for use in generating mutants including deletion mutants. (Camilli et al. (1993) is incorporated by reference herein in its entirety.) Alternatively, the gene replacement protocol described in Biswas et al., J. Bacteriol. 175:3628-3635 (1993), can be used. Other similar methods are known to those of ordinary skill in the art.

A variety of bacterial mutants, and their construction, are described in U.S. patent application Ser. No. 10/883,599, U.S. Patent Publication No. 2004/0197343, U.S. Patent Publication No. 2005/0249748, and U.S. Patent Publication No. 2004/0228877, each of which is incorporated by reference herein in its entirety.

The degree of attenuation in uptake of the attenuated bacteria by non-phagocytic cells need not be an absolute attenuation in order to provide a safe and effective vaccine. In some embodiments, the degree of attenuation is one that provides for a reduction in toxicity sufficient to prevent or reduce the symptoms of toxicity to levels that are not life threatening.

In some embodiments, the Listeria cannot form colonies, replicate, and/or divide. In some embodiments of the invention, the Listeria is attenuated for proliferation relative to parental or wildtype Listeria.

In some embodiments, the attenuated Listeria is killed, but metabolically active (US Patent Pub. No. 2004/0197343 and Brockstedt, et al., Nat. Med., 11:853-60 (2005), incorporated by reference herein in its entirety).

The Listeria, may, in some embodiments, be attenuated by a nucleic acid targeting compound. In some embodiments, the nucleic-acid targeting compound is a nucleic acid alkylator, such as β-alanine, N-(acridin-9-yl), 2-[bis(2-chloroethyl)amino]ethyl ester. In some embodiments, the nucleic acid targeting compound is activated by irradiation, such as UVA irradiation. In some embodiments, the Listeria is treated with a psoralen compound. For instance, in some embodiments, the bacterium are modified by treatment with a psoralen, such as 4′-(4-amino-2-oxa)butyl-4,5′,8-trimethylpsoralen (“S-59”), and UVA light. In some embodiments, the nucleic acid of the bacterium has been modified by treatment with a psoralen compound and UVA irradiation. Descriptions of methods of modifying bacteria to attenuate them for proliferation using nucleic acid targeting compounds are described in U.S. Patent Pub. No. 2004/0197343 and Brockstedt, et al., Nat. Med., 11:853-60 (2005). In some embodiments, the Listeria is attenuated for DNA repair.

For example, for treatment of Listeria such as ΔactAΔuvrAB L. monocytogenes, in some embodiments, S-59 psoralen can be added to 200 nM in a log-phase culture of (approximately) OD₆₀₀=0.5, followed by inactivation with 6 J/m² of UVA light when the culture reaches an optical density of one. Inactivation conditions are optimized by varying concentrations of S-59, UVA dose, the time of S-59 exposure prior to UVA treatment as well as varying the time of treatment during bacterial growth of the Listeria actA/uvrAB strain. The parental Listeria strain is used as a control. Inactivation of Listeria (log-kill) is determined by the inability of the bacteria to form colonies on BHI (Brain heart infusion) agar plates. In addition, one can confirm the continued metabolic activity and expression of proteins such as LLO in the bacteria in the S-59/UVA inactivated Listeria using ³⁵5-pulse-chase experiments to determine the synthesis and secretion of newly expressed proteins post S-59/UVA inactivation. Expression of LLO using ³⁵S-metabolic labeling can be routinely determined. S-59/UVA inactivated Listeria actA/uvrAB can be incubated for 1 hour in the presence of ³⁵S-Methionine. Expression and/or secretion of proteins such as LLO can be determined of both whole cell lysates, and TCA precipitation of bacterial culture fluids. LLO-specific monoclonal antibodies can be used for immunoprecipitation to verify the continued expression and secretion from recombinant Listeria post inactivation.

In some embodiments, the Listeria attenuated for proliferation are also attenuated for nucleic acid repair and/or are defective with respect to at least one DNA repair enzyme. For instance, in some embodiments, the bacterium in which nucleic acid has been modified by a nucleic acid targeting compound such as a psoralen (combined with UVA treatment) is a uvrAB deletion mutant.

In some embodiments, the proliferation of the Listeria is attenuated by at least about 0.3 log, also at least about 1 log, about 2 log, about 3 log, about 4 log, about 6 log, or at least about 8 log. In another embodiment, the proliferation of the Listeria is attenuated by about 0.3 to >10 log, about 2 to >10 log, about 4 to >10 log, about 6 to >10 log, about 0.3-8 log, about 0.3-6 log, about 0.3-5 log, about 1-5 log, or about 2-5 log. In some embodiments, the expression of LLO by the Listeria is at least about 10%, about 25%, about 50%, about 75%, or at least about 90% of the expression of LLO in non-modified Listeria.

V. Pharmaceutical Compositions, Immunogenic Compositions, and/or Vaccines

A variety of different compositions such as pharmaceutical compositions, immunogenic compositions, and vaccines comprising the Listeria described herein are also provided by the invention. In some embodiments, the compositions are isolated.

As used herein, “carrier” includes any and all solvents, dispersion media, vehicles, coatings, diluents, antifungal agents, isotonic and absorption delaying agents, buffers, carrier solutions, suspensions, colloids, and the like. Pharmaceutically acceptable carriers are well known to those of ordinary skill in the art, and include any material which, when combined with an active ingredient, allows the ingredient to retain biological activity and is non-reactive with the subject's immune system. For instance, pharmaceutically acceptable carriers include, but are not limited to, water, buffered saline solutions (e.g., 0.9% saline), emulsions such as oil/water emulsions, and various types of wetting agents. Possible carriers also include, but are not limited to, oils (e.g., mineral oil), dextrose solutions, glycerol solutions, chalk, starch, salts, glycerol, and gelatin.

While any suitable carrier known to those of ordinary skill in the art may be employed in the pharmaceutical compositions, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. In some embodiments, for parenteral administration, such as subcutaneous injection, the carrier comprises water, saline, alcohol, a fat, a wax or a buffer. In some embodiments, any of the above carriers or a solid carrier, such as mannitol, lactose, starch, magnesium stearate, sodium saccharine, talcum, cellulose, glucose, sucrose, and magnesium carbonate, are employed for oral administration.

Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

In addition to pharmaceutical compositions, immunogenic compositions are provided. For instance, the invention provides an immunogenic composition comprising a recombinant bacterium described herein.

In some embodiments, the recombinant bacterium in the immunogenic composition releases the polypeptide comprising the antigen at a level sufficient to induce an immune response to the antigen upon administration of the composition to a host (e.g., a mammal such as a human). In some embodiments, the immune response stimulated by the immunogenic composition is a cell-mediated immune response. In some embodiments, the immune response stimulated by the immunogenic composition is a humoral immune response. In some embodiments, the immune response stimulated by the immunogenic composition comprises both a humoral and cell-mediated immune response.

It can be determined if a particular form of recombinant bacteria (and/or a particular expression cassette) is useful in an immunogenic composition (or as a vaccine) by testing the ability of the recombinant bacteria to stimulate an immune response in vitro or in a model system.

These immune cell responses can be measured by both in vitro and in vivo methods to determine if the immune response of a particular recombinant bacterium (and/or a particular expression cassette) is effective. One possibility is to measure the presentation of the protein or antigen of interest by an antigen-presenting cell that has been mixed with a population of the recombinant bacteria. The recombinant bacteria may be mixed with a suitable antigen presenting cell or cell line, for example a dendritic cell, and the antigen presentation by the dendritic cell to a T cell that recognizes the protein or antigen can be measured. If the recombinant bacteria are expressing the protein or antigen at a sufficient level, it will be processed into peptide fragments by the dendritic cells and presented in the context of MHC class I or class II to T cells. For the purpose of detecting the presented protein or antigen, a T cell clone or T cell line responsive to the particular protein or antigen may be used. The T cell may also be a T cell hybridoma, where the T cell is immortalized by fusion with a cancer cell line. Such T cell hybridomas, T cell clones, or T cell lines can comprise either CD8+ or CD4+ T cells. The dendritic cell can present to either CD8+ or CD4+ T cells, depending on the pathway by which the antigens are processed. CD8+ T cells recognize antigens in the context of MHC class I while CD4+ recognize antigens in the context of MHC class II. The T cell will be stimulated by the presented antigen through specific recognition by its T cell receptor, resulting in the production of certain proteins, such as IL-2, tumor necrosis factor-α (TNF-α), or interferon-γ (IFN-γ), that can be quantitatively measured (for example, using an ELISA assay, ELISPOT assay, or Intracellular Cytokine Staining (ICS)). These are techniques that are well known in the art.

Alternatively, a hybridoma can be designed to include a reporter gene, such as β-galactosidase, that is activated upon stimulation of the T cell hybridoma by the presented antigens. The increase in the production of β-galactosidase can be readily measured by its activity on a substrate, such as chlorophenol red-B-galactoside, which results in a color change. The color change can be directly measured as an indicator of specific antigen presentation.

Additional in vitro and in vivo methods for assessing the antigen expression of recombinant bacteria vaccines of the present invention are known to those of ordinary skill in the art. It is also possible to directly measure the expression of a particular heterologous antigen by recombinant bacteria. For example, a radioactively labeled amino acid can be added to a cell population and the amount of radioactivity incorporated into a particular protein can be determined. The proteins synthesized by the cell population can be isolated, for example by gel electrophoresis or capillary electrophoresis, and the amount of radioactivity can be quantitatively measured to assess the expression level of the particular protein. Alternatively, the proteins can be expressed without radioactivity and visualized by various methods, such as an ELISA assay or by gel electrophoresis and Western blot with detection using an enzyme linked antibody or fluorescently labeled antibody.

Elispot assay, Intracellular Cytokine Staining Assay (ICS), measurement of cytokine expression of stimulated spleen cells, and assessment of cytotoxic T cell activity in vitro and in vivo are all techniques for assessing immunogenicity known to those in the art.

In addition, therapeutic efficacy of the vaccine composition can be assessed more directly by administration of the immunogenic composition or vaccine to an animal model such as a mouse model, followed by an assessment of survival or tumor growth. For instance, survival can be measured following administration of the Listeria and challenge.

Mouse models useful for testing the immunogenicity of an immunogenic composition or vaccine expressing a particular antigen can be produced by first modifying a tumor cell so that it expresses the antigen of interest or a model antigen and then implanting the tumor cells expressing the antigen of interest into mice. The mice can be vaccinated with the candidate immunogenic composition or vaccine comprising a recombinant bacterium expressing a polypeptide comprising the antigen of interest or a model antigen prior to implantation of the tumor cells (to test prophylactic efficacy of the candidate composition) or following implantation of the tumor cells in the mice (to test therapeutic efficacy of the candidate composition).

As an example, CT26 mouse murine colon carcinoma cells can be transfected with an appropriate vector comprising an expression cassette encoding the desired antigen or model antigen using techniques standard in the art. Standard techniques such as flow cytometry and Western blots can then be used to identify clones expressing the antigen or model antigen at sufficient levels for use in the immunogenicity and/or efficacy assays.

Alternatively, candidate compositions can be tested which comprise a recombinant bacterium expressing an antigen that corresponds to or is derived from an antigen endogenous to a tumor cell line (e.g., the retroviral gp70 tumor antigen AH1 endogenous to CT26 mouse murine colon carcinoma cells, or the heteroclitic epitope AH1-A5). In such assays, the tumor cells can be implanted in the animal model without further modification to express an additional antigen. Candidate vaccines comprising the antigen can then be tested.

As indicated, vaccine compositions comprising the bacteria described herein are also provided.

In some embodiments, the vaccine compositions comprise antigen-presenting cells (APC) which have been infected with any of the recombinant bacteria described herein. In some embodiments the vaccine (or immunogenic or pharmaceutical composition) does not comprise antigen-presenting cells (i.e., the vaccine or composition is a bacteria-based vaccine or composition, not an APC-based vaccine or composition).

Methods of administration suitable for administration of vaccine compositions (and pharmaceutical and immunogenic compositions) are known in the art, and include oral, intraveneous, intradermal, intraperitoneal, intramuscular, intralymphatic, intranasal and subcutaneous routes of administration.

Vaccine formulations are known in the art and in some embodiments may include numerous additives, such as preservatives (e.g., thimerosal, 2-phenyoyx ethanol), stabilizers, adjuvants (e.g. aluminum hydroxide, aluminum phosphate, cytokines), antibiotics (e.g., neomycin, streptomycin), and other substances. In some embodiments, stabilizers, such as lactose or monosodium glutamate (MSG), are added to stabilize the vaccine formulation against a variety of conditions, such as temperature variations or a freeze-drying process. In some embodiments, vaccine formulations may also include a suspending fluid or diluent such as sterile water, saline, or isotonic buffered saline (e.g., phosphate buffered to physiological pH). Vaccine may also contain small amount of residual materials from the manufacturing process.

For instance, in some embodiments, the vaccine compositions are lyophilized (i.e., freeze-dried). The lyophilized preparation can be combined with a sterile solution (e.g., citrate-bicarbonate buffer, buffered water, 0.4% saline, or the like) prior to administration.

In some embodiments, the vaccine compositions may further comprise additional components known in the art to improve the immune response to a vaccine, such as adjuvants or co-stimulatory molecules. In addition to those listed above, possible adjuvants include chemokines and bacterial nucleic acid sequences, like CpG. In some embodiments, the vaccines comprise antibodies that improve the immune response to a vaccine, such as CTLA4. In some embodiments, co-stimulatory molecules comprise one or more factors selected from the group consisting of GM-CSF, IL-2, IL-12, IL-14, IL-15, IL-18, B7.1, B7.2, and B7-DC are optionally included in the vaccine compositions of the present invention. Other co-stimulatory molecules are known to those of ordinary skill in the art.

In additional aspects, the invention provides methods of improving a vaccine or immunogenic composition comprising Listeria that express an antigen.

Methods of producing the vaccines of the present invention are also provided.

### VI. Uses

A variety of methods of using the Listeria or pharmaceutical, immunogenic, or vaccine compositions described herein for inducing immune responses, and/or preventing or treating conditions in a host (e.g., a mammal) are provided. In some embodiments, the condition that is treated or prevented is a disease. In some embodiments, the disease is cancer. In some embodiments, the disease is an infectious disease.

As used herein, “treatment” or “treating” (with respect to a condition or a disease) encompasses an approach for obtaining beneficial or desired results. In some embodiments, these results include clinical results. For purposes of this invention, beneficial or desired results with respect to a disease may include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, and/or prolonging survival. Likewise, for purposes of this invention, beneficial or desired results with respect to a condition may include, but are not limited to, one or more of the following: improving a condition, curing a condition, lessening severity of a condition, delaying progression of a condition, alleviating one or more symptoms associated with a condition, increasing the quality of life of one suffering from a condition, and/or prolonging survival. For instance, in those embodiments where the compositions described herein are used for treatment of cancer, the beneficial or desired results may include, but are not limited to, one or more of the following: reducing the proliferation of (or destroying) neoplastic or cancerous cells, reducing metastasis of neoplastic cells found in cancers, shrinking the size of a tumor, decreasing symptoms resulting from the cancer, increasing the quality of life of those suffering from the cancer, decreasing the dose of other medications required to treat the disease, delaying the progression of the cancer, and/or prolonging survival of patients having cancer.

As used herein, the terms “preventing” disease or “protecting a host” from disease (used interchangeably herein) encompass, but are not limited to, one or more of the following: stopping, deferring, hindering, slowing, retarding, and/or postponing the onset or progression of a disease, stabilizing the progression of a disease, and/or delaying development of a disease. The terms “preventing” a condition or “protecting a host” from a condition (used interchangeably herein) encompass, but are not limited to, one or more of the following: stopping, deferring, hindering, slowing, retarding, and/or postponing the onset or progression of a condition, stabilizing the progression of a condition, and/or delaying development of a condition. The period of this prevention can be of varying lengths of time, depending on the history of the disease or condition and/or individual being treated. By way of example, where the vaccine is designed to prevent or protect against an infectious disease caused by a pathogen, the terms “preventing” disease or “protecting a host” from disease encompass, but are not limited to, one or more of the following: stopping, deferring, hindering, slowing, retarding, and/or postponing the infection by a pathogen of a host, progression of an infection by a pathogen of a host, or the onset or progression of a disease associated with infection of a host by a pathogen, and/or stabilizing the progression of a disease associated with infection of a host by a pathogen. Also, by way of example, where the vaccine is an anti-cancer vaccine, the terms “preventing” disease or.“protecting the host” from disease encompass, but are not limited to, one or more of the following: stopping, deferring, hindering, slowing, retarding, and/or postponing the development of cancer or metastasis, progression of a cancer, or a reoccurrence of a cancer.

In one aspect, the invention provides a method of inducing an immune response in a host (e.g., mammal) to an antigen, comprising administering to the host an effective amount of a bacterium described herein or an effective amount of a composition (e.g., a pharmaceutical composition, immunogenic composition, or vaccine) comprising a bacterium described herein.

In some embodiments, the immune response is an MHC Class I immune response. In other embodiments, the immune response is an MHC Class II immune response. In still other embodiments, the immune response that is induced by administration of the bacteria or compositions is both an MHC Class I and an MHC Class II response. Accordingly, in some embodiments, the immune response comprises a CD4+ T-cell response. In some embodiments, the immune response comprises a CD8+ T-cell response. In some embodiments, the immune response comprises both a CD4+ T-cell response and a CD8+ T-cell response. In some embodiments, the immune response comprises a B-cell response and/or a T-cell response. B-cell responses may be measured by determining the titer of an antibody directed against the antigen, using methods known to those of ordinary skill in the art. In some embodiments, the immune response which is induced by the compositions described herein is a humoral response. In other embodiments, the immune response which is induced is a cellular immune response. In some embodiments, the immune response comprises both cellular and humoral immune responses. In some embodiments, the immune response is antigen-specific. In some embodiments, the immune response is an antigen-specific T-cell response.

In addition to providing methods of inducing immune responses, the present invention also provides methods of preventing or treating a condition or disease in a host (e.g., a mammalian subject such as human patient). The methods comprise administration to the host of an effective amount of a bacterium described herein, or a composition comprising a bacterium described herein. In some embodiments, the disease is cancer. In some embodiments, the disease is an infectious disease.

In some embodiments, the disease is cancer. In some embodiments, where the condition being treated or prevented is cancer, the disease is melanoma, breast cancer, pancreatic cancer, liver cancer, colon cancer, colorectal cancer, lung cancer, brain cancer, testicular cancer, ovarian cancer, squamous cell cancer, gastrointestinal cancer, cervical cancer, kidney cancer, thyroid cancer or prostate cancer. In some embodiments, the cancer is melanoma. In some embodiments, the cancer is pancreatic cancer. In some embodiments, the cancer is colon cancer. In some embodiments, the cancer is prostate cancer. In some embodiments, the cancer is metastatic.

In other embodiments, the disease is an infectious disease or another disease caused by a pathogen such as a virus, bacterium, fungus, or protozoa. In some embodiments, the disease is an infectious disease.

In some embodiments, the use of the Listeria in the prophylaxis or treatment of a cancer comprises the delivery of the Listeria to cells of the immune system of an individual to prevent or treat a cancer present or to which the individual has increased risk factors, such as environmental exposure and/or familial disposition. In other embodiments, the use of the bacteria in the prophylaxis or treatment of a cancer comprises delivery of the bacteria to an individual who has had a tumor removed or has had cancer in the past, but is currently in remission.

In some embodiments, administration of composition comprising a bacterium described herein to a host elicits a CD4+ T-cell response in the host. In some other embodiments, administration of a composition comprising a bacterium described herein to a host elicits a CD8+ T-cell response in the host. In some embodiments, administration of a composition comprising a bacterium described herein elicits both a CD4+ T-cell response and a CD8+ T-cell response in the host.

The efficacy of the vaccines or other compositions for the treatment of a condition can be evaluated in an individual, for example in mice. A mouse model is recognized as a model for efficacy in humans and is useful in assessing and defining the vaccines of the present invention. The mouse model is used to demonstrate the potential for the effectiveness of the vaccines in any individual. Vaccines can be evaluated for their ability to provide either a prophylactic or therapeutic effect against a particular disease. For example, in the case of infectious diseases, a population of mice can be vaccinated with a desired amount of the appropriate vaccine of the invention, where the bacterium expresses an infectious disease associated antigen. The mice can be subsequently infected with the infectious agent related to the vaccine antigen and assessed for protection against infection. The progression of the infectious disease can be observed relative to a control population (either non vaccinated or vaccinated with vehicle only or a bacterium that does not contain the appropriate antigen).

In the case of cancer vaccines, tumor cell models are available, where a tumor cell line expressing a desired tumor antigen can be injected into a population of mice either before (therapeutic model) or after (prophylactic model) vaccination with a composition comprising a bacterium of the invention containing the desired tumor antigen. Vaccination with a bacterium containing the tumor antigen can be compared to control populations that are either not vaccinated, vaccinated with vehicle, or with a bacterium that expresses an irrelevant antigen. The effectiveness of the vaccine in such models can be evaluated in terms of tumor volume as a function of time after tumor injection or in terms of survival populations as a function of time after tumor injection. In one embodiment, the tumor volume in mice vaccinated with a composition comprising the bacterium is about 5%, about 10%, about 25%, about 50%, about 75%, about 90% or about 100% less than the tumor volume in mice that are either not vaccinated or are vaccinated with vehicle or a bacterium that expresses an irrelevant antigen. In another embodiment, this differential in tumor volume is observed at least about 10, about 17, or about 24 days following the implant of the tumors into the mice. In one embodiment, the median survival time in the mice vaccinated with the composition comprising a bacterium is at least about 2, about 5, about 7 or at least about 10 days longer than in mice that are either not vaccinated or are vaccinated with vehicle or bacteria that express an irrelevant antigen.

The host (i.e., subject) in the methods described herein, is any vertebrate, preferably a mammal, including domestic animals, sport animals, and primates, including humans. In some embodiments, the host is a mammal. In some embodiments, the host is a human.

The delivery of the Listeria, or a composition comprising the strain, may be by any suitable method, such as intradermal, subcutaneous, intraperitoneal, intravenous, intramuscular, intralymphatic, oral or intranasal, as well as by any route that is relevant for any given malignant or infectious disease or other condition. In some embodiments, the method of administration is mucosal.

The compositions comprising the bacteria and an immunostimulatory agent may be administered to a host simultaneously, sequentially or separately. Examples of immunostimulatory agents include, but are not limited to IL-2, IL-12, GMCSF, IL-15, B7.1, B7.2, and B7-DC and IL-14. Additional examples of stimulatory agents are provided in Section V, above

As used herein, an “effective amount” of a bacterium or composition (such as a pharmaceutical composition or an immunogenic composition) is an amount sufficient to effect beneficial or desired results. For prophylactic use, beneficial or desired results includes results such as eliminating or reducing the risk, lessening the severity, or delaying the outset of the disease, including biochemical, histologic and/or behavioral symptoms of a disease, its complications and intermediate pathological phenotypes presenting during development of the disease. For therapeutic use, beneficial or desired results includes clinical results such as inhibiting or suppressing a disease, decreasing one or more symptoms resulting from a disease (biochemical, histologic and/or behavioral), including its complications and intermediate pathological phenotypes presenting during development of a disease, increasing the quality of life of those suffering from a disease, decreasing the dose of other medications required to treat the disease, enhancing effect of another medication, delaying the progression of the disease, and/or prolonging survival of patients. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an effective amount may be considered in the context of administering one or more therapeutic agents, and a single agent may be considered to be given in an effective amount if, in conjunction with one or more other agents, a desirable result may be or is achieved.

In some embodiments, for a therapeutic treatment of a cancer, an effective amount includes an amount that will result in the desired immune response, wherein the immune response either slows the growth of the targeted tumors, reduces the size of the tumors, or preferably eliminates the tumors completely. The administration of the vaccine may be repeated at appropriate intervals, and may be administered simultaneously at multiple distinct sites in the vaccinated individual. In some embodiments, for a prophylactic treatment of a cancer, an effective amount includes a dose that will result in a protective immune response such that the likelihood of an individual to develop the cancer is significantly reduced. The vaccination regimen may be comprised of a single dose, or may be repeated at suitable intervals until a protective immune response is established.

In some embodiments, the therapeutic treatment of an individual for cancer may be started on an individual who has been diagnosed with a cancer as an initial treatment, or may be used in combination with other treatments. For example, individuals who have had tumors surgically removed or who have been treated with radiation therapy or by chemotherapy may be treated with the vaccine in order to reduce or eliminate any residual tumors in the individual, or to reduce the risk of a recurrence of the cancer. In some embodiments, the prophylactic treatment of an individual for cancer, would be started on an individual who has an increased risk of contracting certain cancers, either due to environmental conditions or genetic predisposition.

The dosage of the pharmaceutical compositions or vaccines that are given to the host will vary depending on the species of the host, the size of the host, and the condition or disease of the host. The dosage of the compositions will also depend on the frequency of administration of the compositions and the route of administration. The exact dosage is chosen by the individual physician in view of the patient to be treated.

In some embodiments, a single dose of the pharmaceutical compositions, immunogenic compositions, or vaccines comprising the Listeria described herein comprises from about 10² to about 10¹² of the bacterial organisms. In another embodiment, a single dose comprises from about 10⁵ to about 10¹¹ of the bacterial organisms. In another embodiment, a single dose comprises from about 10⁶ to about 10¹¹ of the bacterial organisms. In still another embodiment, a single dose of the pharmaceutical composition or vaccine comprises from about 10⁷ to about 10¹⁰ of the bacterial organisms. In still another embodiment, a single dose of the pharmaceutical composition or vaccine comprises from about 10⁷ to about 10⁹ of the bacterial organisms.

The Listeria of the present invention, in some embodiments, is administered in a dose, or dosages, where each dose comprises at least about 1000 Listeria units/kg body weight, at least about 10,000 Listeria units/kg body weight, at least about 100,000 Listeria units/kg body weight, at least about 1 million Listeria units/kg body weight, or at least about 10 million.Listeria units/kg body weight. The present invention provides the above doses where the units of Listeria are colony forming units (CFU), the equivalent of CFU prior to psoralen-treatment, or where the units are number of Listeria cells. In some embodiments, the effective amount of attenuated Listeria that is measured comprises at least about 1 x 10³ CFU/kg or at least about 1×10³ Listeria cells/kg. In some embodiments, the effective amount of attenuated Listeria that is measured comprises at least about 1×10⁵ CFU/kg or at least about 1×10⁵ Listeria cells/kg. In certain embodiments, the effective amount of attenuated Listeria that is measured comprises at least about 1×10⁶ CFU/kg or at least about 1×10⁶ Listeria cells/kg. In some embodiments, the effective amount of attenuated Listeria that is measured comprises at least about 1×10⁷ CFU/kg or at least about 1×10⁷ Listeria cells/kg. In some further embodiments, the effective amount of attenuated Listeria that is measured comprises at least about 1×10⁸ CFU/kg or at least about 1>10⁸ Listeria cells/kg.

In some embodiments, a single dose of the pharmaceutical composition, immunogenic composition, or vaccine comprising the Listeria described herein comprises from about 1 CFU/kg to about 1×10¹⁰ CFU/kg (CFU=colony forming units). In some embodiments, a single dose of the composition comprises from about 10 CFU/kg to about 1×10⁹ CFU/kg. In another embodiment, a single dose of the composition or vaccine comprises from about 1×10² CFU/kg to about 1×10⁸ CFU/kg. In still another embodiment, a single dose of the composition or vaccine comprises from about 1×10³ CFU/kg to about 1×10⁸ CFU/kg. In still another embodiment, a single dose of the composition or vaccine comprises from about 1×10⁴ CFU/kg to about 1×10⁷ CFU/kg. In some embodiments, a single dose of the composition comprises at least about 1 CFU/kg. In some embodiments, a single dose of the composition comprises at least about 10 CFU/kg. In another embodiment, a single dose of the composition or vaccine comprises at least about 1×10² CFU/kg. In still another embodiment, a single dose of the composition or vaccine comprises at least about 1×10³ CFU/kg. In still another embodiment, a single dose of the composition or vaccine comprises from at least about 1×10⁴ CFU/kg.

In some embodiments, the proper (i.e., effective) dosage amount for one host, such as human, may be extrapolated from the LD₅₀ data for another host, such as a mouse, using methods known to those in the art.

In some embodiments, the pharmaceutical composition, immunogenic composition, or vaccine comprises antigen-presenting cells, such as dendritic cells, which have been infected with the Listeria described herein. In some embodiments, an individual dosage of an antigen-presenting cell based vaccine comprising bacteria such as those described herein comprises between about 1×10³ to about 1×10¹⁰ antigen-presenting cells. In some embodiments, an individual dosage of the vaccine comprises between about 1×10⁵ to about 1×10⁹ antigen-presenting cells. In some embodiments, an individual dosage of the vaccine comprises between about 1×10⁷ to about 1×10⁹ antigen-presenting cells.

In some embodiments, multiple administrations of the dosage unit are preferred, either in a single day or over the course of a week or month or year or years. In some embodiments, the dosage unit is administered every day for multiple days, or once a week for multiple weeks. In some embodiments, the Listeria are administered to the mammalian subject at least twice, at least three times, at least four times, at least five times, at least 10 times, or at least 20 times.

The invention also provides a method of inducing MHC class I antigen presentation or MHC class II antigen presentation on an antigen-presenting cell comprising contacting a bacterium described herein with an antigen-presenting cell.

The invention further provides a method of inducing an immune response in a host to an antigen comprising, the following steps: (a) contacting a Listeria bacterium described herein with an antigen-presenting cell from the host, under suitable conditions and for a time sufficient to load the antigen-presenting cells; and (b) administering the antigen-presenting cell to the host.

### VII. Kits

The invention further provides kits and articles of manufacture comprising the Listeria described herein, or compositions comprising the Listeria described herein.

### Examples

### General Methods.

Intracellular staining (ICS) assays involve permeabilizing the splenocytes, and treating with an antibody that binds cytokines that have accumulated inside the immune cell, where the antibody allows fluorescent tagging. Brefeldin blocks protein transport, and provokes the accumulation of cytokines within the immune cell.

Elispot (enzyme-linked immunospot) assays are sensitive to secreted proteins, where the proteins are secreted over a period of time from immune cells resting in a well. A capture antibody is bound to the well, which immobilized secreted cytokine. After the secretory period, the cells are removed, and a detection antibody is used to detect immobilized cytokine. The capture antibody and detection antibody bind to different regions of the cytokine. Methodological details of the ICS and elispot assays are disclosed (see, e.g., U.S. Pat. Appl. Pub. No. 2005/0249748, published Nov. 10, 2005, of Dubensky, et al.).

### Example One

### Schematic Diagrams Showing Transit of a Macromolecule from Listeria to Host Cell Cytoplasm, as Mediated by Holin or Holin Plus Lysin. Construction of Plasmids Containing Expression Cassettes Encoding Holin, Lysin, or Holin and Lysin

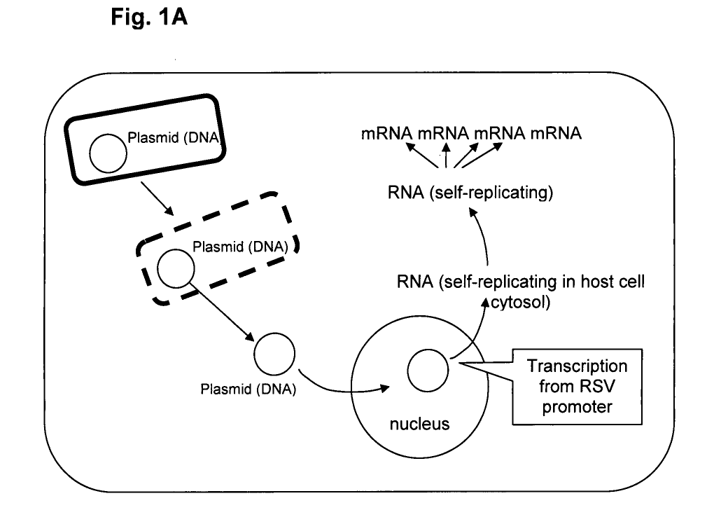

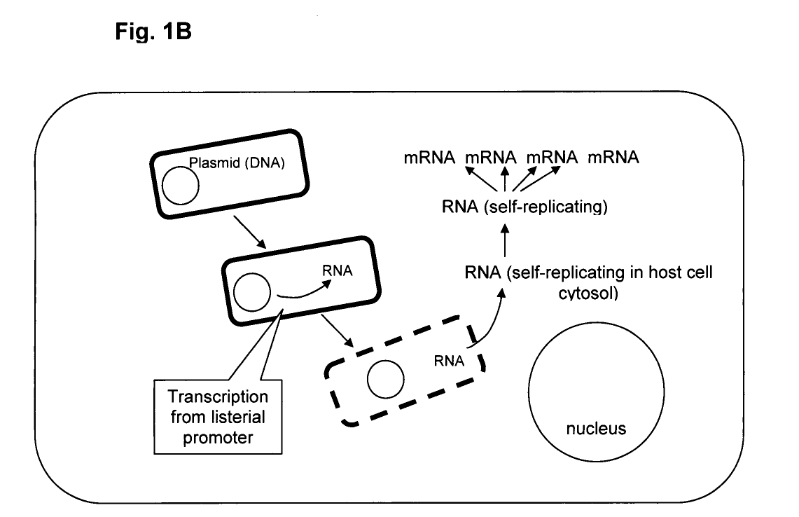

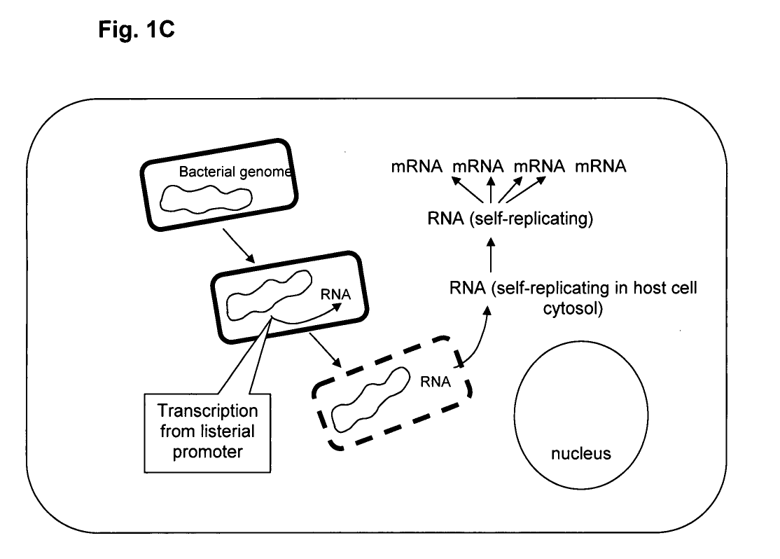

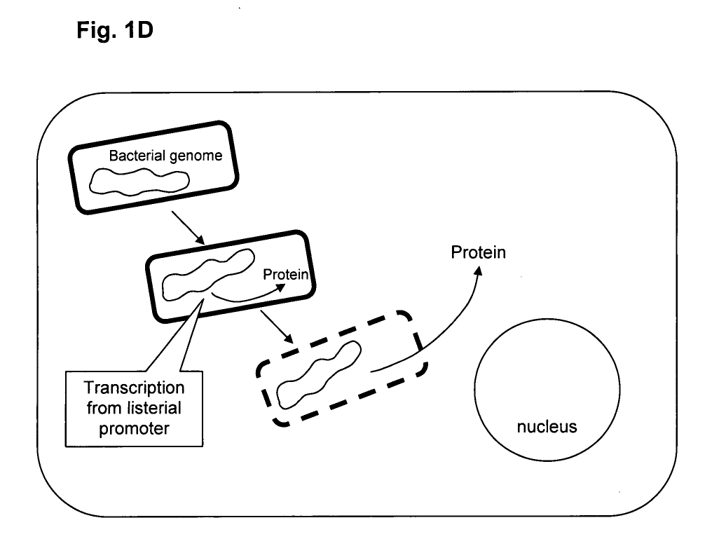

The schematic diagrams of FIGS. 1A to 1D disclose a number of non-limiting embodiments of the nucleic acids and Listeria bacteria of the invention.

FIG. 1A discloses a Listeria bacterium harboring a plasmid. The plasmid contains a virus-derived expression cassette. Release of the plasmid from the bacterium can be mediated by holin, holin without any recombinant lysin, the combination of holin and a recombinant lysin, and the like. Once in the nucleus, transcription machinery of the host cell can use a mammalian cell compatible promoter(s), and transcribe a self-amplifying (replicating) expression cassette, where replication is mediated by proteins encoded by the expression cassette. Host cell proteins may contribute to expression and/or post-translational modification of protein encoded by the virus-derived expression cassette. The amplified mRNA encodes the desired heterologous protein, e.g., a tumor antigen or infectious agent antigen.

In another aspect, the plasmid that is released from the bacterium can encode a conventional transcription unit, that is, a transcription unit that is not a self-amplifying virus-derived expression cassette.

The dashes represent any degree of permeabilization as mediated in whole or in part by the expressed holin and/or lysin. The dashes can also represent lysis, as mediated in whole or in part by the expressed holin and/or lysin.

FIG. 1B also discloses a Listeria bacterium harboring a plasmid. The plasmid contains a virus-derived expression cassette, where a Listeria-compatible promoter(s) is operably linked with the expression cassette. RNA transcribed from the expression cassette is shown in the figure to be released from the bacterium, where release can be mediated by holin, holin without any recombinant lysin, the combination of holin and a recombinant lysin, and the like.

Once in the cytoplasm, self-amplification (replication) is mediated by proteins encoded by the expression cassette. Host cell proteins may contribute to expression and/or post-translational modification of protein encoded by the virus-derived expression cassette. The amplified mRNA encodes the desired heterologous protein, e.g., a tumor antigen or infectious agent antigen.

FIG. 1C shows a Listeria bacterium harboring a genome-based virus-derived expression cassette, where a Listeria-compatible promoter(s) is operably linked with the expression cassette. RNA transcribed from the expression cassette is shown in the figure to be released from the bacterium, where release can be mediated by holin, holin without any recombinant lysin, the combination of holin and a recombinant lysin, and the like. As disclosed above, once in the cytoplasm of the host cell, self-amplification occurs, where the amplified RNA encodes the desired heterologous protein(s).

FIG. 1D discloses the release of polypeptides from the permeabilized or lysed Listeria bacterium. The polypeptide can be short peptide, a typical polypeptide of about 20-200 kD, or it can be a very large polypeptide or complex of polypeptides. In some embodiments, what is encompassed by the invention is a nucleic acid encoding the polypeptide, where there does not exist any Listeria-compatible secretory sequence. In some embodiments, what is also encompassed is a nucleic acid encoding the polypeptide, where the polypeptide contains a Listeria-compatible secretory sequence, and so on.

The figure discloses that the polypeptide is encoded by a genome-based nucleic acid, however plasmid-based nucleic acids are also contemplated. Release of the polypeptide from the bacterium can be mediated by a holin, a holin without any recombinant lysin, by the combination of holin and a recombinant lysin, and the like.

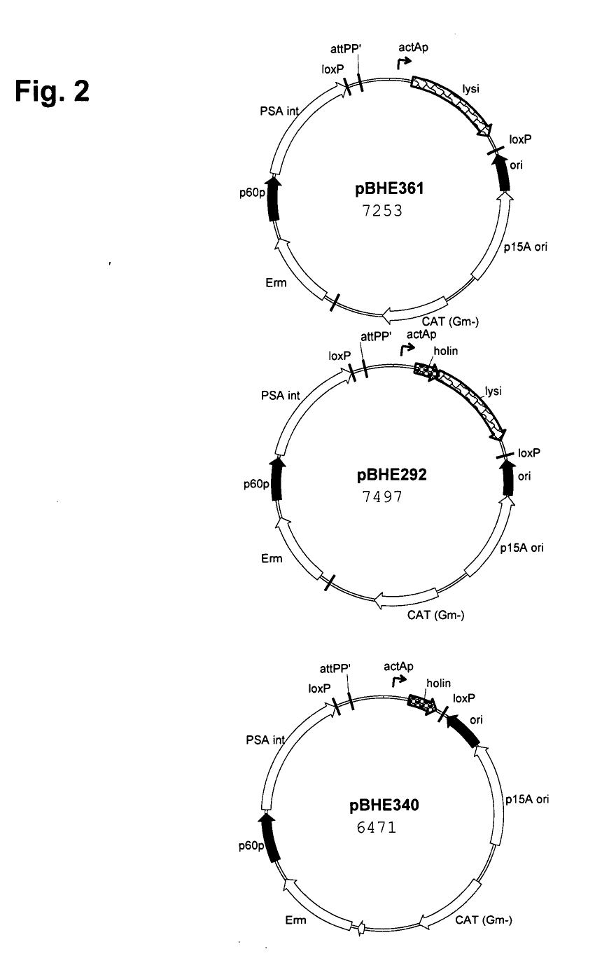

FIG. 2 discloses plasmids containing expression cassettes containing nucleic acids encoding holin, lysin, or holin and lysin. The nucleic acids are operably linked with actA promoter, a promoter that is specifically activated with the Listeria bacterium is in a host cell. The plasmids contain an attPP′ site, which is a short nucleic acid sequence used to mediate site-specific integration into a target nucleic acid, where the target nucleic acid contains a corresponding attBB′ site. Generally, the plasmid is transfected into a Listeria bacterium, and the target is an attBB′ site in the listerial genome. A number of attPP′ sites, corresponding attBB′ sites, and integrases that catalyze the integration, are available (see, e.g., Lauer, et al. (2002) J. Bacteriol. 183:4177-4186; ENGINEERED LISTERIA AND METHODS OF USE THEREOF, U.S. Ser. No. 11/395,197 (filed Mar. 30, 2006), assigned to Cerus Corporation).

The figure also discloses that the plasmids contain a pair of loxP sites. After site-specific integration has occurred, the loxP sites can be used to mediate elimination of the intervening nucleic acid, that is, the nucleic acid encoding an antibiotic resistance gene or other selection marker gene. Cre recombinase, introduced into the bacteria by way of a plasmid, is an enzyme that recognizes loxP sites and catalyzes elimination of the nucleic acid (e.g., antibiotic gene) that is flanked by the loxP sites.

Assembly of the plasmids and nucleic acids of FIG. 2 is disclosed below.

The polycistron containing the holin and lysin ORFs were amplified by PCR with bacteriophage PSA (Zimmer, et al. (2003) Mol. Microbiol. 50, 303-317) genomic DNA as template and using the following primers:

PL529 (forward):

(SEQ ID NO: 23)

5′ ttGGATTCPatgaaaattaactggaaagt 3′
PL530 (reverse):

(SEQ ID NO: 24)

5′ ttGAGCTCGGCCGCGGCCGCagtatgaggaagtggaacgt 3′

After amplification, the PCR product was purified over a Qiagen® column, eluted and digested with ClaI and EagI. After complete digestion, the fragment was cloned into pINT (ENGINEERED LISTERIA AND METHODS OF USE THEREOF, U.S. Ser. No. 11/395,197, filed Mar. 30, 2006, and assigned to Cerus Corporation) cut with the same set of restriction enzymes downstream of the actA promoter, resulting in pBHE292.

The plasmid expressing lysin only was engineered in a similar manner. The lysin ORF was PCR amplified with PSA genomic DNA as template using the following primers:

PL612 (forward):

(SEQ ID NO: 25)

5′ AAAATCGATATGATAGTAATGAGTAATTATAGTATGTCG 3′
PL613 (reverse):

(SEQ ID NO: 26)

5′ AAAGCGGCCGCAGTATGAGGAAGTGGAACGTATGTACTTAT 3′

After amplification, the PCR products was purified over a Qiagen® column, eluted and digested with ClaI and EagI. The fragment was cloned into pINT downstream of the actA promoter resulting in pBHE361.

The plasmid expressing holin only was derived from pBHE292 by deletion of the lysin sequence from the unique NruI site to the unique NotI. After restriction digest, the plasmid was blunted using T4 ploymerase, purified over a Qiagen® column and self ligated. This resulted in pBHE340.

After engineering the three plasmids and confirming their fidelity by sequence analysis, they were integrated at the tRNA^{Arg} locus in the genome of selected Listeria strains using previously described methods (Lauer, et al. (2002) J. Bacteriol. 184, 4177-4186). Integration was confirmed on erythromycin resistant Listeria colonies by PCR with NC16 (5′ gtcaaaacatacgctcttatc 3′) and PL95 (5′ acataatcagtccaaagtagatgc 3′).

### Example Two

### Derivation of Recombinant L. monocytogenes (Lm) Strains Containing Holin, Lysin or Holin and Lysin Expression Cassettes and Characterization of their Growth Properties in Broth and in Mammalian Host Cells

Listeria monocytogenes (Lm) was engineered to contain a nucleic acid encoding a listeriophage holin (PSA phage), listeriophage lysin (PSA phage), or both holin and lysin. Site-specific integration was at the attBB′ site naturally occurring at the tRNA^{Arg} locus of the listerial genome, were transcription was controlled by the listerial actA promoter, a promoter specifically activated by conditions inside a host cell.

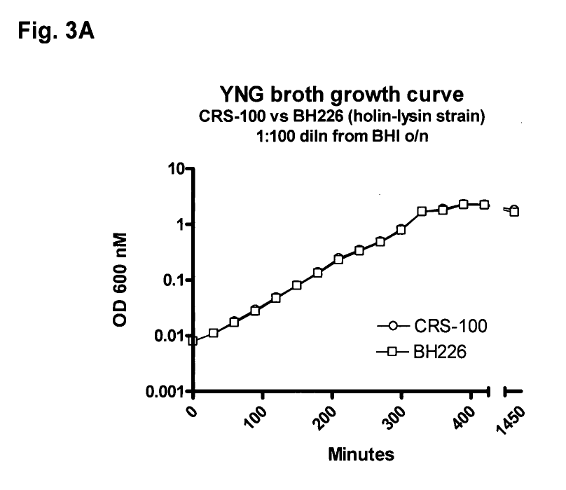

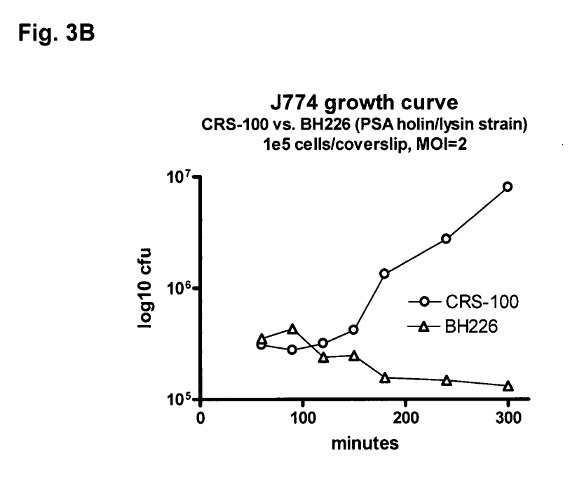

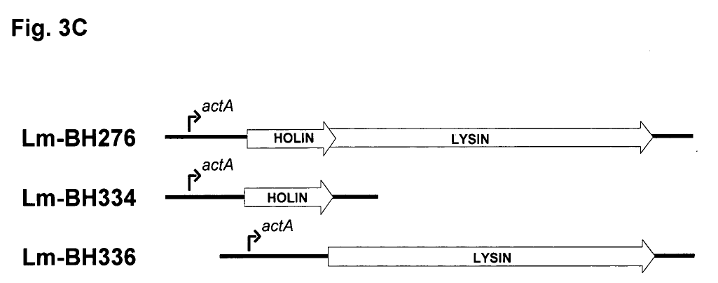

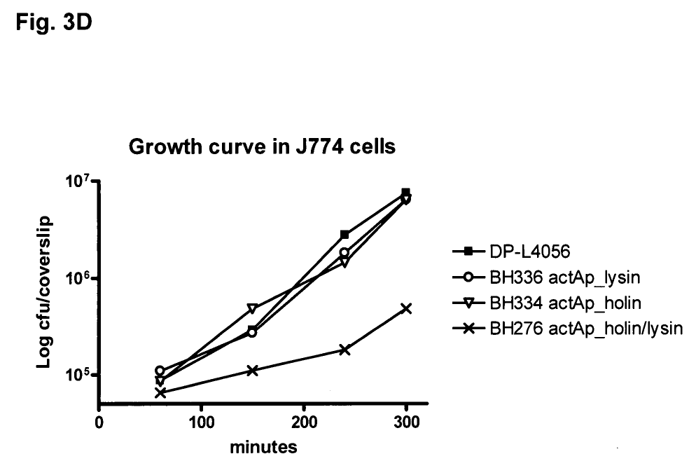

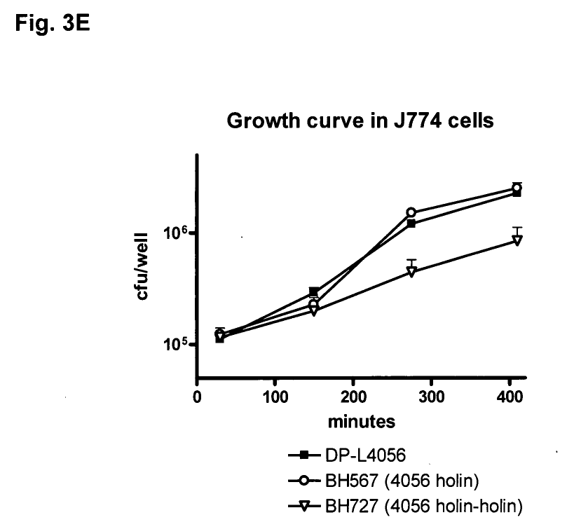

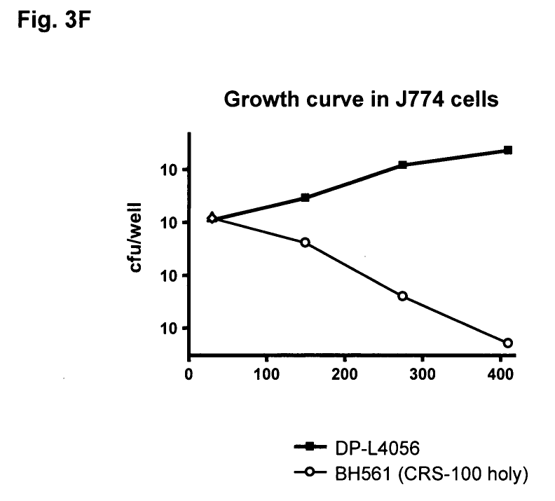

FIG. 3A demonstrates that growth of the parental Lm strain (“CRS-100”) and of Lm-holin-lysin were the same in broth. Bacterial viability of LmΔactAΔinlB (“CRS-100”) and BH226 was determined at 7 hrs and 24 hrs and was comparable at both time points for both Listeria strains (˜5×10⁹ cfu/ml). Therefore, the holin-lysin cassette is not detrimental to the growth of the bacteria in broth culture. The identical growth curves demonstrate lack of expression of holin and lysin with culture in broth, even though Lm-holin-lysin was engineered to contain nucleic acids encoding these two proteins. BH226 is attenuated for growth in mammalian host cells compared to the parental Listeria strain (FIG. 3B).

FIG. 3C is a schematic diagram of the listerial constructs containing a polypeptide comprising a nucleic acid encoding holin, a nucleic acid encoding lysin, or nucleic acids encoding holin and lysin. Expression was from the actA promoter. The parental Lm strain, “CRS-100,” is Lm ΔactAΔinlB, a strain of Lm that is attenuated by deletions in the actA gene and inlB gene. FIG. 3D demonstrates that expression of both holin and lysin (BH276) are required to inhibit intracellular growth of L. monocytogenes in mammalian cells (J774 cells). In contrast, L. monocytogenes strains that express only holin (BH334) or only lysin (BH336) are not inhibited in intracellular bacterial growth in J774 cells as compared to wild-type L. monocytogenes (DP14056).

FIG. 3E demonstrates that expression of only holin (and with no expression of lysin) does not necessarily impair growth of Lm when Lm is grown inside host mammalian cells (J774 cells). The figure discloses near-identical intracellular growth curves of Lm-holin and parental Lm, and somewhat lesser growth of a listerial construct expressing two copies of the holin gene (Lm-holin-holin). One copy of holin does not affect growth or bacterial viability in host cells (BH567), whereas two copies of the actA promoter-holin cassette does affect the growth and viability of the bacteria in host cells (BH727). FIG. 3F, repeat of growth attenuation of holin+lysin containing bacteria where growth attenuation is dramatic. Repeated experiments demonstrated that Lm-holin showed no consistent difference in intracellular growth, when compared with that of a control parental Lm not containing a nucleic acid encoding holin. In other words, the holin expressed by Lm-holin did not inhibit intracellular growth.

The invention contemplates a Lm-holin capable of sustained growth and/or metabolism, where the sustained growth and/or metabolism results in continued expression of nucleic acids encoding a heterologous antigen. While not limiting the invention to any particular property or advantage, a contemplated advantage is as follows. Lm-holin containing a nucleic acid encoding an antigen, or a nucleic acid encoding a viral-derived expression cassette, is contemplated to show sustained expression of the nucleic acid, while the expressed holin mediates transit of the expressed antigen (or the expressed viral-derived expression cassette) from the bacterium into the host cell's cytosol.

FIG. 3F discloses lesser growth properties of Lm expressing both holin and lysin (Lm-holin-lysin; Lm-holy). The figure discloses a rapid rate of disappearance of viable bacteria, in the case of Lm-holin-lysin and, in contrast, steady growth of the parental Listeria strain.

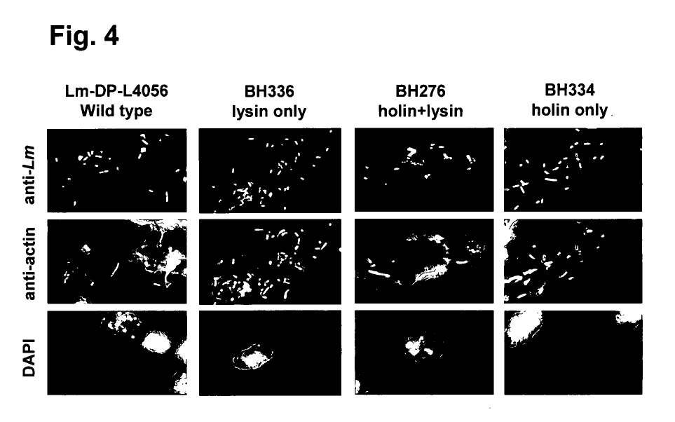

FIG. 4 provides photographs where bacteria, actin, and dsDNA were visualized by anti-Lm antibodies, anti-actin antibodies, and diamidinophenylindole (DAPI), respectively. DAPI staining results in visualization of bacteria by way of staining the bacterial genome, and it also visualizes the mammalian host cell's nucleus. DAPI and anti-0 antigen antibodies are described (see, e.g., (see, e.g., Hazeleger, et al. (2006) Int. J. Food Microbiol. June 23 epub; Aarnisalo, et al. (2003) J. Food Prot. 66:249-255). Anti-actin antibodies for staining are available (see, e.g., Amersham Pharmacia Biotech, Piscataway, NJ; Sigma Aldrich, St. Louis, Mo.).

The figure demonstrates that expression of both holin and lysin by Lm-holin-lysin results in fragmentation of the Listeria bacteria, failure to show the expected actin trails. Fragmentation was demonstrated with the anti-O antigen antibodies, which stain an extracellular marker on Listeria. Fragmentation and destruction of the bacteria was confirmed by DAPIs failure to stain bacteria. In contrast, parental Lm, Lm-lysin, and Lm-holin, showed equivalent staining by anti-O antigen antibodies.

These results, which show that holin alone did not fragment bacteria under the conditions of the experiment, are consistent with those shown above, demonstrating that parental Lm and Lm-holin can show similar or identical growth rates.

The failure of lysin alone, as expressed by Lm-lysin, to fragment the bacteria suggests that Lm-lysin cannot serve as a suitable vehicle for mediating transfer of nucleic acids, viral-based expression cassettes, polypeptides, from the inside of a Listeria bacterium to an external environment, e.g., a host cell cytoplasm.

In other words, the immunofluorescence images of J774 cells infected with various Listeria strains show the following. For the three strains DP-L4056, BH334, and BH336, the bacteria appear as wild type—that is, intact and associated with actin tails. In the case of BH276, while some bacteria are associated with actin tails, there are several instances of “exploded bacteria”, where the anti-Listeria antibody recognizes fragments of bacteria. This is visual confirmation of the growth curve data.

Further details for EXAMPLE TWO were as follows.

Strains were constructed by conjugation from E. coli to L. monocytogenes essentially as described (Lauer, et al. (2002) J. Bacteriol. 184:4177-4186). Holin and/or lysin cassettes were introduced to wild-type (DP-L4056), ΔactA, ΔuvrAB, or ΔactAΔinlB strain backgrounds.

Growth curves were done in broth culture starting from a stationary phase overnight 3 ml VPP (Veggie Peptone Phosphate, Oxoid). A 1:100 dilution was done in VPP or YNG (Yeast no glucose) media and OD 600 nm readings were taken at 30 to 60 minute time intervals. Viable bacteria in a culture were determined by plating appropriate serial dilutions on VPP plates and counting colonies.

Growth curves in J774 cells were done essentially as described (Portnoy, et al. (1988) J. Exp. Med. 167:1459-1471). Briefly, J774 cells were maintained in DMEM+10% FBS and antibiotic and seeded in either 6 well dishes with 12 mm coverslips or into 24 well plates without coverslips at a density of 1e6 cells per ml. The next day, cells were washed with PBS, resuspended in media without antibiotic, and infected with a fresh 30 ° C. overnight of the appropriate Listeria strain at a multiplicity of infection (moi) of 1, 2, 5, or 10. After 30 or 60 minutes, cells were washed with PBS and media was replaced with DMEM containing 50 μg/ml of Gentamycin. Growth was monitored by sampling at various time points in triplicate by lysing host cells in water and plating appropriate dilutions on VPP.

Immunofluorescence was done essentially as described (Skoble, et al. (2000) J. Cell Biol. 150:527-538). Infections were performed as above on 12 mm coverslips, and cells were fixed in 3.5% formaldehyde. Bacteria were stained with anti-listeria O antigen polycolonal antibody (Difco) and visualized with a secondary antibody conjugated to FITC. Actin was stained with Rhodamine phalloidin. DAPI stained the nuclei of host cells and DNA of the bacteria and was present in the mounting media (Molecular Probes, Eugene, Oreg.).

### Example Three

### Utility of Recombinant L. monocytogenes (Lm) Strains Expressing Holin, Lysin, or Holin and Lysin, to Deliver a Eukaryotic Expression Plasmid to the Cytoplasm of a Host Cell

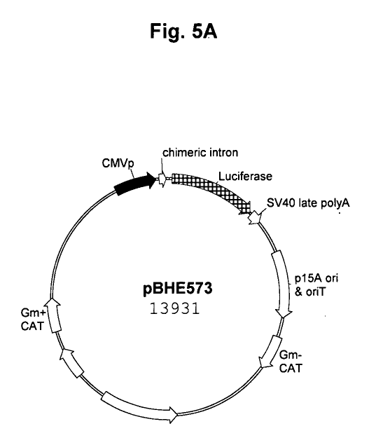

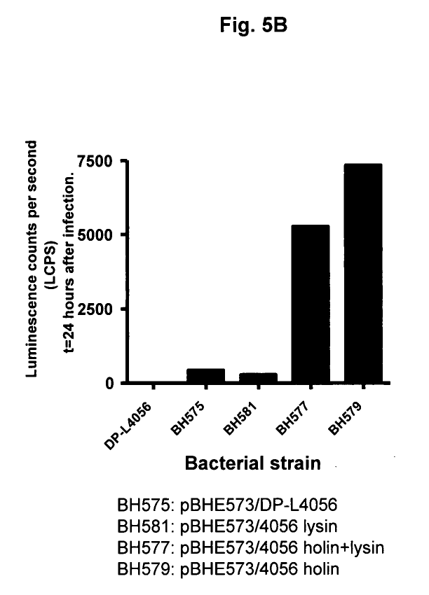

FIG. 5A is a schematic diagram showing a plasmid, suitable for placing in a Listeria bacterium, for subsequent release, and for monitoring release by way of activity of expressed luciferase. The plasmid encodes luciferase operably linked with a promoter compatible with mammalian host cell transcription machinery.

FIG. 5B compares release of the luciferase-encoding plasmid from Listeria to the cytoplasm of BHK cells. Luciferase-catalyzed luminescence generated by infecting host cells with Listeria monocytogenes (Lm) constructs containing DNA plasmid encoding luciferase. Lm constructs were parental Lm (no plasmid), parental Lm (+plasmid); Lm-lysin (+plasmid); Lm-holin-lysin (+plasmid); and Lm-holin (+plasmid). Luciferase-catalyzed luminescence, as shown by luminescence counts per second (LCPS), was determined under two conditions, that is, where the indicated Lm was added to 1×10⁵ BHK cells.

The figure demonstrates that lysin alone, as expressed by Lm-lysin, failed to stimulate release of plasmid from the bacterium, as compared to the control bacterium containing the plasmid but no holin and no lysin. In contrast, holin alone, as expressed by Lm-holin, produced significant luminescence, for example, 7,500 counts per second. The combination of holin and lysin, as expressed by Lm-holin-lysin, also produced significant luminescence.

To conclude, expression of holin alone, or holin with lysin, resulted in readily measurable release of the plasmid from the bacterium, while expression of lysin alone did not result in any detectable release.

These results are consistent with those noted above in the bacterial fragmentation experiments. Failure of lysin alone, as expressed by Lm-lysin, to release luciferase-plasmid from Listeria monocytogenes suggests that Lm-lysin cannot serve as a suitable vehicle for mediating transfer of nucleic acids, viral-based expression cassettes, polypeptides, and the like, from the inside of a Listeria bacterium to an external environment, e.g., to a mammalian host cell cytoplasm.

Construction of the relevant plasmids, nucleic acids, and Listeria strains is disclosed below.

A plasmid was constructed allowing maintenance in Listeria as well as expression of luciferase in host cells. The backbone of this plasmid was derived from pAM401 (Wirth, et al. (1986) J. Bacteriol. 165:831-836). In order to make this plasmid conducive to bacterial conjugation, the oriT from pPL2 (Lauer, et al. (2002) J. Bacteriol. 184:4177-4186) was amplified by PCR and cloned into the unique SacII site resulting in pAM401oriT. The initial luciferase plasmid was constructed by digesting pAM401oriT with EagI and BamHI, treating with CIP (NEB) and purifying over a Qiagen® column. The luciferase cassette was obtained by digesting pGL3 control vector (Promega, Madison, Wis.) with NotI and BamHI and gel purifying the 2609 by fragment. The two fragments were ligated together using T4 DNA ligase (NEB) and colonies confirmed by PCR and restriction enzyme digest. This resulted in the mammalian expression vector pBHE539. After initial experiments demonstrated lower than desired luminescence values, the SV40 promoter was replaced with a region of the pRL-CMV vector (Promega, Madison, Wis.) containing the CMV enhancer and immediate early promoter as well as a chimeric intron. The plasmid pBHE539 was digested with BglII and StuI, treated with CIP and cleaned up over a Qiagen® column. The CMV promoter region was obtained by digesting pRL-CMV with BglII and ScaI and gel purifying the 1035 by fragment. The fragments were ligated together using T4 DNA ligase and clones confirmed by PCR and restriction digest resulting in pBHE573. When compared in mammalian cell infection experiments, pBHE573 resulted in luminescence values 10-20 fold higher than pBHE539.

To test the utility of the various holin and/or lysin expressing Listeria strains for delivery of a eukaryotic expression plasmid to host cells, the vector pBHE573 was moved into the various holin/lysin strains by conjugation as described previously (Lauer, et al. (2002) J. Bacteriol. 184, 4177-4186). The day before cell infection, strains harboring the plasmid were grown in BHI medium+10 ug/ml chloramphenicol (for maintenance of plasmid) at 30° C. for 16 hrs. BHK cells (ref) were maintained in a T75 flask containing 15 mls DMEM+10% FBS+1xNEAA+50 mg/L penicillin-streptomycin at 37° C/5% CO₂. Cells at circa 90% confluence were treated with 3 mL trypsin solution for 15 minutes at 37° C. Cells were then diluted with 10 mls DMEM+10% FBS+1xNEAA, pelleted and resuspended in 3 mls DMEM+10% FBS+1xNEAA. After cell number was determined, culture was diluted to 3 E⁵ cells/ml and transferred to 12-well plates (1 ml culture/well) for overnight incubation at 37° C/5% CO₂ (results in one cell doubling). The day of infection, one ml of bacterial culture at a concentration of 2 E9 bacteria/ml was pelleted and resuspended in 1 ml DPBS (Mediatech) for each strain. This bacterial suspension was used to inoculate DMEM+10% FBS+1xNEAA to a final concentration of 6 E⁷ bacteria/ml (MOI of 100). Medium was removed from the BHK cells by aspiration and replaced with 1 ml of bacterial suspension. Cells were infected for 1 hour at 37° C./5% CO₂ After infection, the medium was removed by aspiration and replaced with DMEM+10% FBS+1xNEAA+50 mg/L gentamicin. Cells were then incubated overnight at 37° C./5% CO₂. After 24 hrs, cells were assayed for luciferase activity using the Steady-Glo Luciferase Assay System (Promega, Madison, Wis.). Medium was removed by aspiration and replaced with 220 ul cell lysis buffer containing luciferin. After incubation of cells for 10 mins at RT, two 100 ul aliquots/well were transferred to a 96-well plate for reading in a luminometer (Perkin-Elmer Trilux).

### Example Four

### Utility of Recombinant L. monocytogenes (Lm) Strains Expressing Holin, Lysin, or Holin and Lysin, for Delivering a Plasmid DNA Replicon to the Cytoplasm of a Mammalian Host Cell

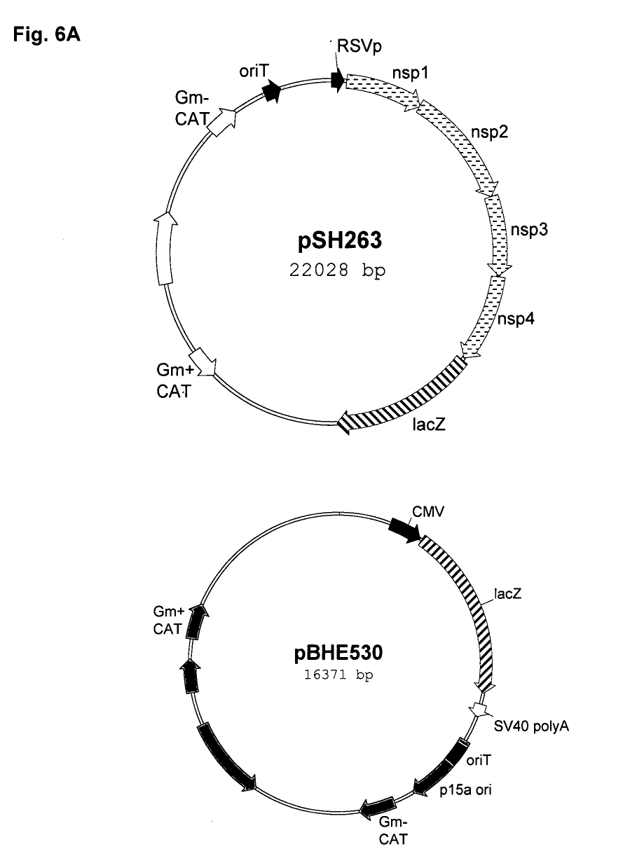

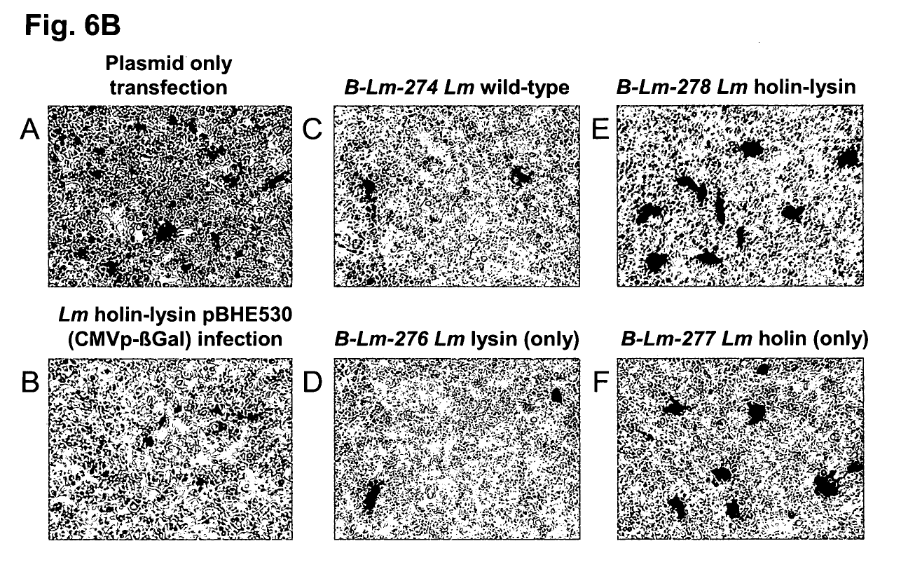

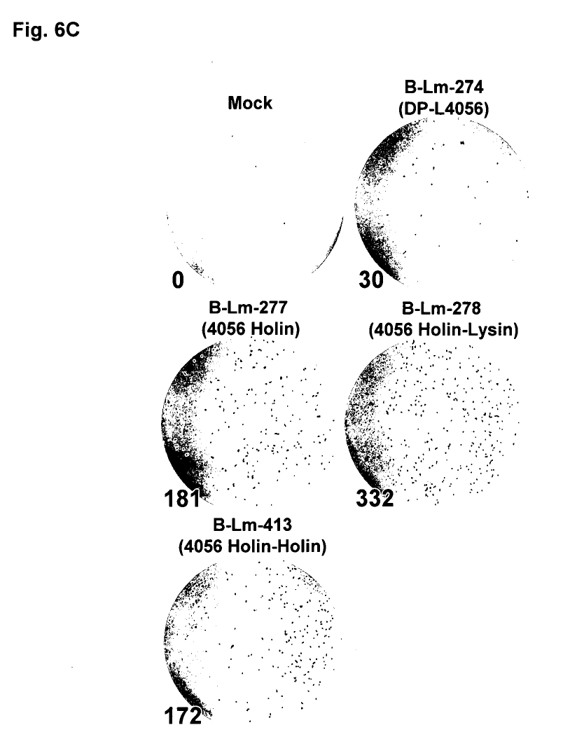

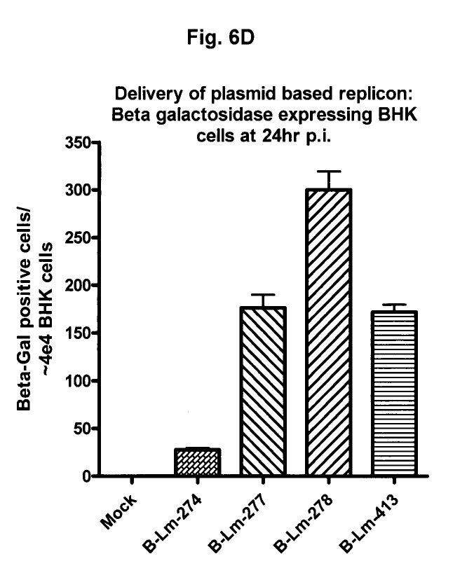

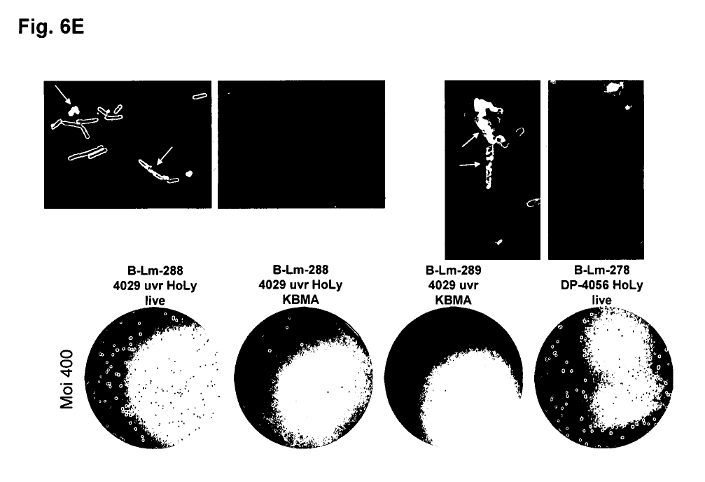

FIG. 6A discloses a plasmid, pSH263, containing a nucleic acid that is an alphavirus-based expression cassette, where the expression cassette contains a nucleic acid encoding β-galactosidase (lacZ gene). The non-structural genes encoded by the expression cassette mediate self-amplification (replication) of the plasmid, where replication of the expression cassette occurs after the plasmid leaves the Listeria bacterium for the mammalian host cell's cytosol.

FIGS. 6B-6E disclose biological results where the indicated Lm constructs were administered to mammalian cells. The mammalian cells were BHK cells or J774 cells, as indicated. As indicated, the Lm constructs were parental Lm (Lm wild-type); Lm holin-lysin (Lm-HoLy); Lm-lysin; and Lm-holin. “Lm-Ho-Ho” means Listeria monocytogenes containing two copies of a nucleic acid encoding holin.

Other listerial constructs were based on KBMA Lm ΔuvrAB, a preparation of Listeria monocytogenes mutated in a DNA repair gene (uvrAB) and treated with an agent (psoralen) that cross-links the listerial genome. KBMA Lm ΔuvrAB is metabolically active however its genome contains a small number of chemical cross-links, and the bacterium is metabolically active but is not able to form colonies (see, e.g., Brockstedt, et al. (2005) Nature Medicine 11:853-860; U.S. Pub. No. US 2004/0197343 of Dubensky, et al.). The KBMA-based constructs included KBMA Lm ΔuvrAB-holin-lysin (KBMA Lm ΔuvrAB-HoLy).

FIG. 6B shows light microscope pictures of BHK cells treated with the indicated vector. BHK cells were infected with Listeria containing a plasmid encoding an alphavirus-derived expression cassette. β-galactosidase expression was measured in BHK cells at 24 hr. following introduction of pSH263 plasmid alphavirus expression cassette (pAM402oriT pSin-lacZ). A positive signal (dark spots) indicates expression of β-galactosidase (Panels A-F).

Panel A discloses that transfecting BHK cells with the plasmid (virus-based replicon) resulted in a high degree of expression. (Here, the plasmid was not inside a Listeria bacterium.

Panel B shows that β-galactosidase expression from a different type of plasmid, pBHE530 (FIG. 6A), a plasmid where expression is controlled by a conventional promoter (CMV promoter). (This plasmid does not contain a viral-based replicon, and there is no self-amplification of message in the cytoplasm.) Expression was low.

Panel C shows expression using wild type Lm (no holin; no lysin) containing the plasmid encoding the virus-based replicon. β-galactosidase expression was low.

Panel D shows expression using Lm-lysin containing the virus-based replicon. Expression was also low. The low degree of expression using Lm-lysin, a degree similar to that of parental Lm (no holin; no lysin), demonstrates that lysin alone does not mediate release of a plasmid from Lm. In other words, β-galactosidase expression from Lm-lysin was similar to background.

Panel E demonstrates that expression using Lm-holin-lysin containing the virus-based replicon results in a relatively high degree of β-galactosidase expression. Panel F demonstrates that similar high degrees of β-galactosidase expression were found where Lm-holin was used. To summarize, various Lm constructs were administered to BHK cells, where the Lm contained a plasmid bearing a self-amplifying virus-based expression cassette. Where the Lm construct was Lm-holin or Lm-holin-lysin, the expressed holin mediated release of the plasmid out of the bacterium to the host cell's cytosol, where plasmid-encoded enzymes could amplify the expression cassette, and where the mammalian translational machinery could express large amounts of heterologous antigen (β-galactosidase). The results showed that lysin alone did not mediate plasmid release.

FIG. 6C discloses a similar experiment using BHK cells, as disclosed by photographs of BHK cells containing various Lm constructs, and FIG. 6D discloses histograms quantitating the raw data. As indicated, BHK cells were treated with buffer only (mock); parental Lm (DP-L4056); Lm-holin; Lm-holin-lysin (Lm-HoLy); and Lm-holin-holin (Lm-Ho-Ho).

FIG. 6E shows a similar experiment as above, but with J774 cells instead of BHK cells. The bacterial constructs were: live Lm ΔuvrAB-holin-lysin (Lm ΔuvrAB-HoLy); KBMA Lm ΔuvrAB-holin-lysin (Lm ΔuvrAB-HoLy); KBMA Lm ΔuvrAB; and live Lm-holin-lysin (Lm-HoLy). The term “live” means that psoralen had not been added to convert the “live” bacteria to killed but metabolically active (KBMA) bacteria. The fluorescent photomicrographs demonstrate the fragmentation of the Listeria bacteria, where the bacteria expressed both holin and lysin. The light microscope photographs reveals the signal generated by β-galactosidase biosynthesized in the cytoplasm of the host J774 cells, where the β-galactosidase is encoded by the plasmid DNA replicon. β-galactosidase activity, which is dependent on both release of the plasmid from the bacterium, amplification in the J774 cell's cytosol, and translation, was greatest with live Lm-holin-lysin and with Lm ΔuvrAB-holin-lysin. Significant β-galactosidase activity was also found with KBMA Lm ΔuvrAB-holin-lysin.

In other words, the top photographs show two examples of KBMA bacteria lysing within the host cell in a Holin-Lysin dependent manner. For each panel, the left is stained with the anti-Listeria polyclonal antibody and visualized with FITC. Bacteria that are lysin are noted with an arrow in each panel. The right is stained with rhodamine-phalloidin. The bottom photographs show the following. Holin-Lysin KBMA Listeria bacteria are able to deliver to Sindbis virus-lacZ replicon to BHK. The first and last panel are live Holin-Lysin control strains, panel two is KBMA holin-lysin with the replicon, panel three is KBMA without holin-lysin.

To conclude, the data demonstrates the utility of a virus-derived expression cassette, as provided by Lm-holin vector, in expressing a heterologous antigen.

Details for EXAMPLE FOUR were as follows. Construction of pSH263 was as described. The shuttle plasmid pSH263 was constructed from a Sindbis viral replicon and a lacZ reporter cloned downstream of an RSV promoter for intracellular DNA launch. The Sindbis virus replicon, lacZ and the RSV promoter components of pSH263 were derived from the plasmids Sinrep/lacZ and Sinrep21 from Dr. Sondra Schlesinger of Washington University School of Medicine, St. Louis, Mo. A BamHI-EagI fragment containing the RSV promoter and a portion of nonstructural proteins was cut from Sinrep21 and inserted into BamHI-EagI sites of pSH252 to create the plasmid pSH258. The remaining nonstructural proteins and a lacZ reporter were cut from Sinrep/lacZ as a BglII-NotI fragment and cloned into the BglII-EagI sites of pSH258. The resulting plasmid, pSH263 was used to transform E. coli strain SM10 resulting in strain B-Ec-266. The plasmid pSH263 was transferred from B-Ec-266 to various Listeria strains by conjugation. The construction of pSH263 was confirmed by EcoRI digestion and the integrity of the replicon confirmed by transient transfection of BHK cells followed by staining for β-galactosidase activity as described below.

The plasmid pSH252 was derived from pAM401 (Wirth, et al. (1986) J. Bacteriol. 165:831-836) by inserting the oriT from pINT into the SacII site of pAM401. The oriT from pINT was amplified with the following primers that include SacII sites (underlined) using Platinum Pfx polymerase according to product instructions:

WL214

(SEQ ID NO: 27)

5′ acatCCGCGGTTTCAGTGCAATTTATCTCTTCAAATG 3′
WL215

(SEQ ID NO: 28)

5′ atctCCGCGGATGTATGCTATACGAAGTTATGCG 3′

The resulting PCR product was digested with SacII and inserted into SacII-digested pAM401. The resulting plasmid, pSH252, replicates as an episome in both E. coli and Listeria hosts and is transferred from E. coli donors to Listeria hosts by conjugation as described previously.

As a positive control for DNA delivery to eukaryotic cells by Listeria, the strain BH276 (pBHE530) was derived. This strain expresses holin and lysin, and contains the plasmid pBHE530.

The plasmid pBHE530 was constructed as follows. As a positive control for DNA delivery to eukaryotic cells by Listeria, the strain BH276(pBHE530) was derived. This strain expresses holin and lysin and contains the plasmid pBHE530. The plasmid pBHE530 was constructed by subcloning the CMV immediate-early promoter and lacZ gene from the plasmid pShuttle-CMV-lacZ (Stratagene, San Diego, Calif.) into the plasmid pSH252. The CMV promoter-lacZ expression cassette was removed from pShuttle-CMV-lacZ by SapI/SacII digestion, gel purified and blunted with T4 DNA polymerase. The 3473 bp fragment was inserted into the EcoRV site of pSH252 to complete construction of pBHE530. The plasmid pBHE530 was used to transform E. coli SM10 and the Listeria strain BH276(pBHE530) completed by subsequent conjugation.

Transient transfections in BHK cells were performed to confirm that the Sindbis virus replicon could be launched from pSH263 in eukaryotic cells. BHK cells were seeded in 6-well plates at a density of 5×10⁵ cells/well in complete growth medium (DMEM contianing 10% fetal calf serum and non-essential amino acids) and cultured overnight at 37° C. Prior to transfection, monolayers were 90-100% confluent and washed twice in complete growth medium. Four micrograms of pSH263 DNA were diluted in 250 uL OptiMEM (Invitrogen Corp.) and mixed with 20 μL Lipofectamine 2000® (Invitrogen Corp.) diluted in 250 μL OptiMEM according to product instructions. Following a 20 minute incubation at room temperature, 500 μL DNA-lipofectamine mixture was added directly to each well of BHK cells in 2 ml complete grown medium. Transfected cells were cultured at 37° C. and stained for β-galactosidase activity at 24 hours post-transfection.

β-Galactosidase activity staining was performed using a procedure modified from Sanes, et al. (1986) EMBO J. 5:3133-3142, and published on the Invitrogen web site. Briefly, transfected or infected BHK cells were washed with PBS and fixed lightly in 2% (v/v) formaldehyde, 0.2% (v/v) glutaraldehyde in PBS for 5 minutes at room temperature, washed with PBS then stained overnight with 1 mg/mL X-gal in 5 mM potassium ferricyanide, 5 mM potassium ferrocyanide, 2 mM magnesium chloride and 1× PBS. After staining cells were rinsed with PBS and fixed for 10 minutes at room temperature in 10% (v/v) formaldehyde in PBS. Fixed cells were washed once with PBS then stored at 4° C. in PBS until analysis.

In order to investigate the ability of Listeria strains expressing holin, lysin, or holin and lysin to deliver Sindbis virus replicons to infected BHK cells, the plasmid pSH263 was introduced into the Listeria strains DP-L4056, BH334, BH336, BH276 and BH727 by conjugation with the E. coli donor strain B-Ec-266 resulting in the strains B-Lm-274, B-Lm-277, B-Lm-276, B-Lm-278 and B-Lm-414 respectively. As a positive control for DNA delivery to BHK cells, an SM10 strain containing pBHE530 was conjugated with the Listeria strain BH276 to make BH276(pBHE530). Six-well plates were seeded with 4×10⁵ BHK/well and 96-well plates seeded with 2×10⁴ cell/well in complete growth medium and cultured at 37° C. overnight. All Listeria strains were cultured in BHI containing 10 ug/mL chloramphenicol overnight at 30° C. without shaking. Overnight Listeria cultures were washed twice with PBS and used to infect BHK cells at a multiplicity of infection (MOI) of 200 in 500 uL serum-free DMEM. After 1 hour at 37° C., inocula were aspirated and cells washed three times in serum-free DMEM containing 50 ug/mL gentamycin. Infected cells were cultured in complete growth medium with 50 ug/mL gentamycin and stained for β-galactosidase activity at 24 hours post-transfection.

An experiment with Killed But Metabolically Active Listeria monocytogenes (KBMA Lm) was conducted as follows. To investigate the ability of photochemically inactivated Listeria to deliver pSH263 to eukaryotic cells, the following strains were made. The uvrAB locus was deleted from the Listeria strain DP-L4029 resulting in 4029uvr. Listeria strain 4029uvr was conjugated with E. coli SM10 harboring the plasmid pBHE292 resulting the Listeria strain B-Lm-284. B-Lm-284 contains both holin and lysin downstream of the actA promoter integrated at the tRNA-Arg locus. Next, pSH263 was transferred into B-Lm-284 and 4029uvr by conjugation with B-Ec-266 resulting in the strains B-Lm-288 and B-Lm-289, respectively. Photochemical inactivation of Listeria containing the uvrAB deletion has been described previously. Briefly, overnight cultures of B-Lm-288 and B-Lm-289 were grown at 37° C. in filter-sterilized BHI. Overnight cultures were diluted 1:50 in fresh filter-sterilized BHI and incubated at 37° C. at 300 ron to OD₆₀₀=0.5. A 50 ml aliquot of each culture was transferred to a fresh glass flask and S-59 was added to a concentration of 200 nM and grown at 37° C. at 300 rpm for one hour to reach an approximately OD₆₀₀=1.0. Cultures were transferred to a 100 mm polystyrene petri dish and UVA irradiated at 6 J/cm². S-59 treated Listeria were collected by centrifugation at 2300×g for 20 minutes at 4° C. and washed once with 50 mL PBS. The final pellet was suspended in PBS and used immediately to infect BHK cells. Inactivation was confirmed by plating serial dilutions of Listeria cultures on non-selective medium before and after UVA irradiation.

Six-well plates were seeded with 4×10⁵ BHK/well and cultured overnight at 37° C. Cells were infected at MOI of 200 and 400 with inactivated B-Lm-288 and B-Lm-289 as described earlier. Infected BHK were cultured 24 hours at 37° C. in complete medium containing 50 ug/mL gentamycin then stained for β-galactosidase activity as described above.

The sequence of pSH263 is shown below. The sequence contains an RSV promoter, non-structural proteins 1-4 (nsp104), and lacZ reporter sequence:

(SEQ ID NO: 29)
ggatccagtcttatgcaatactcttgtagtcttgcaacatggtaacgatgagttagcaacat
gccttacaaggagagaaaaagcaccgtgcatgccgattggtggaagtaaggtggtacgatcg
tgccttattaggaaggcaacagacgggtctgacatggattggacgaaccactgaattccgca
ttgcagagatattgtatttaagtgccctacctcgataccgtcgagattgacggcgtagtaca
cactattgaatcaaacagccgaccaattgcactaccatcacaatggagaagccagtagtaaa
cgtagacgtagacccccagagtccgtttgtcgtgcaactgcaaaaaagcttcccgcaatttg
aggtagtagcacagcaggtcactccaaatgaccatgctaatgccagagcattttcgcatctg
gccagtaaactaatcgagctggaggttcctaccacagcgacgatcttggacataggcagcgc
accggctcgtagaatgttttccgagcaccagtatcattgtgtctgccccatgcgtagtccag
aagacccggaccgcatgatgaaatacgccagtaaactggcggaaaaagcgtgcaagattaca
aacaagaacttgcatgagaagattaaggatctccggaccgtacttgatacgccggatgctga
aacaccatcgctctgctttcacaacgatgttacctgcaacatgcgtgccgaatattccgtca
tgcaggacgtgtatatcaacgctcccggaactatctatcatcaggctatgaaaggcgtgcgg
accctgtactggattggcttcgacaccacccagttcatgttctcggctatggcaggttcgta
ccctgcgtacaacaccaactgggccgacgagaaagtccttgaagcgcgtaacatcggacttt
gcagcacaaagctgagtgaaggtaggacaggaaaattgtcgataatgaggaagaaggagttg
aagcccgggtcgcgggtttatttctccgtaggatcgacactttatccagaacacagagccag
cttgcagagctggcatcttccatcggtgttccacttgaatggaaagcagtcgtacacttgcc
gctgtgatacagtggtgagttgcgaaggctacgtagtgaagaaaatcaccatcagtcccggg
atcacgggagaaaccgtgggatacgcggttacacacaatagcgagggcttcttgctatgcaa
agttactgacacagtaaaaggagaacgggtatcgttccctgtgtgcacgtacatcccggcca
ccatatgcgatcagatgactggtataatggccacggatatatcacctgacgatgcacaaaaa
cttctggttgggctcaaccagcgaattgtcattaacggtaggactaacaggaacaccaacac
catgcaaaattaccttctgccgatcatagcacaagggttcagcaaatgggctaaggagcgca
aggatgatcttgataacgagaaaatgctgggtactagagaacgcaagcttacgtatggctgc
ttgtgggcgtttcgcactaagaaagtacattcgttttatcgcccacctggaacgcagacctg
cgtaaaagtcccagcctcttttagcgcttttcccatgtcgtccgtatggacgacctctttgc
ccatgtcgctgaggcagaaattgaaactggcattgcaaccaaagaaggaggaaaaactgctg
caggtctcggaggaattagtcatggaggccaaggctgcttttgaggatgctcaggaggaagc
cagagcggagaagctccgagaagcacttccaccattagtggcagacaaaggcatcgaggcag
ccgcagaagttgtctgcgaagtggaggggctccaggcggacatcggagcagcattagttgaa
accccgcgcggtcacgtaaggataatacctcaagcaaatgaccgtatgatcggacagtatat
cgttgtctcgccaaactctgtgctgaagaatgccaaactcgcaccagcgcacccgctagcag
atcaggttaagatcataacacactccggaagatcaggaaggtacgcggtcgaaccatacgac
gctaaagtactgatgccagcaggaggtgccgtaccatggccagaattcctagcactgagtga
gagcgccacgttagtgtacaacgaaagagagtttgtgaaccgcaaactataccacattgcca
tgcatggccccgccaagaatacagaagaggagcagtacaaggttacaaaggcagagcttgca
gaaacagagtacgtgtttgacgtggacaagaagcgttgcgttaagaaggaagaagcctcagg
tctggtcctctcgggagaactgaccaaccctccctatcatgagctagctctggagggactga
agacccgacctgcggtcccgtacaaggtcgaaacaataggagtgataggcacaccggggtcg
ggcaagtcagctattatcaagtcaactgtcacggcacgagatcttgttaccagcggaaagaa
agaaaattgtcgcgaaattgaggccgacgtgctaagactgaggggtatgcagattacgtcga
agacagtagattcggttatgctcaacggatgccacaaagccgtagaagtgctgtacgttgac
gaagcgttcgcgtgccacgcaggagcactacttgccttgattgctatcgtcaggccccgcaa
gaaggtagtactatgcggagaccccatgcaatgcggattcttcaacatgatgcaactaaagg
tacatttcaatcaccctgaaaaagacatatgcaccaagacattctacaagtatatctcccgg
cgttgcacacagccagttacagctattgtatcgacactgcattacgatggaaagatgaaaac
cacgaacccgtgcaagaagaacattgaaatcgatattacaggggccacaaagccgaagccag
gggatatcatcctgacatgtttccgcgggtgggttaagcaattgcaaatcgactatcccgga
catgaagtaatgacagccgcggcctcacaagggctaaccagaaaaggagtgtatgccgtccg
gcaaaaagtcaatgaaaacccactgtacgcgatcacatcagagcatgtgaacgtgttgctca
cccgcactgaggacaggctagtgtggaaaaccttgcagggcgacccatggattaagcagccc
actaacatacctaaaggaaactttcaggctactatagaggactgggaagctgaacacaaggg
aataattgctgcaataaacagccccactccccgtgccaatccgttcagctgcaagaccaacg
tttgctgggcgaaagcattggaaccgatactagccacggccggtatcgtacttaccggttgc
cagtggagcgaactgttcccacagtttgcggatgacaaaccacattcggccatttacgcctt
agacgtaatttgcattaagtttttcggcatggacttgacaagcggactgttttctaaacaga
gcatcccactaacgtaccatcccgccgattcagcgaggccggtagctcattgggacaacagc
ccaggaacccgcaagtatgggtacgatcacgccattgccgccgaactctcccgtagatttcc
ggtgttccagctagctgggaagggcacacaacttgatttgcagacggggagaaccagagtta
tctctgcacagcataacctggtcccggtgaaccgcaatcttcctcacgccttagtccccgag
tacaaggagaagcaacccggcccggtcaaaaaattcttgaaccagttcaaacaccactcagt
acttgtggtatcagaggaaaaaattgaagctccccgtaagagaatcgaatggatcgccccga
ttggcatagccggtgcagataagaactacaacctggctttcgggtttccgccgcaggcacgg
tacgacctggtgttcatcaacattggaactaaatacagaaaccaccactttcagcagtgcga
agaccatgcggcgaccttaaaaaccctttcgcgttcggccctgaattgccttaacccaggag
gcaccctcgtggtgaagtcctatggctacgccgaccgcaacagtgaggacgtagtcaccgct
cttgccagaaagtttgtcagggtgtctgcagcgagaccagattgtgtctcaagcaatacaga
aatgtacctgattttccgacaactagacaacagccgtacacggcaattcaccccgcaccatc
tgaattgcgtgatttcgtccgtgtatgagggtacaagagatggagttggagccgcgccgtca
taccgcaccaaaagggagaatattgctgactgtcaagaggaagcagttgtcaacgcagccaa
tccgctgggtagaccaggcgaaggagtctgccgtgccatctataaacgttggccgaccagtt
ttaccgattcagccacggagacaggcaccgcaagaatgactgtgtgcctaggaaagaaagtg
atccacgcggtcggccctgatttccggaagcacccagaagcagaagccttgaaattgctaca
aaacgcctaccatgcagtggcagacttagtaaatgaacataacatcaagtctgtcgccattc
cactgctatctacaggcatttacgcagccggaaaagaccgccttgaagtatcacttaactgc
ttgacaaccgcgctagacagaactgacgcggacgtaaccatctattgcctggataagaagtg
gaaggaaagaatcgacgcggcactccaacttaaggagtctgtaacagagctgaaggatgaag
atatggagatcgacgatgagttagtatggattcatccagacagttgcttgaagggaagaaag
ggattcagtactacaaaaggaaaattgtattcgtacttcgaaggcaccaaattccatcaagc
agcaaaagacatggcggagataaaggtcctgttccctaatgaccaggaaagtaatgaacaac
tgtgtgcctacatattgggtgagaccatggaagcaatccgcgaaaagtgcccggtcgaccat
aacccgtcgtctagcccgcccaaaacgttgccgtgcctttgcatgtatgccatgacgccaga
aagggtccacagacttagaagcaataacgtcaaagaagttacagtatgctcctccacccccc
ttcctaagcacaaaattaagaatgttcagaaggttcagtgcacgaaagtagtcctgtttaat
ccgcacactcccgcattcgttcccgcccgtaagtacatagaagtgccagaacagcctaccgc
tcctcctgcacaggccgaggaggcccccgaagttgtagcgacaccgtcaccatctacagctg
ataacacctcgcttgatgtcacagacatctcactggatatggatgacagtagcgaaggctca
cttttttcgagctttagcggatcggacaactctattactagtatggacagttggtcgtcagg
acctagttcactagagatagtagaccgaaggcaggtggtggtggctgacgttcatgccgtcc
aagagcctgcccctattccaccgccaaggctaaagaagatggcccgcctggcagcggcaaga
aaagagcccactccaccggcaagcaatagctctgagtccctccacctctcttttggtggggt
atccatgtccctcggatcaattttcgacggagagacggcccgccaggcagcggtacaacccc
tggcaacaggccccacggatgtgcctatgtctttcggatcgttttccgacggagagattgat
gagctgagccgcagagtaactgagtccgaacccgtcctgtttggatcatttgaaccgggcga
agtgaactcaattatatcgtcccgatcagccgtatcttttccactacgcaagcagagacgta
gacgcaggagcaggaggactgaatactgactaaccggggtaggtgggtacatattttcgacg
gacacaggccctgggcacttgcaaaagaagtccgttctgcagaaccagcttacagaaccgac
cttggagcgcaatgtcctggaaagaattcatgccccggtgctcgacacgtcgaaagaggaac
aactcaaactcaggtaccagatgatgcccaccgaagccaacaaaagtaggtaccagtctcgt
aaagtagaaaatcagaaagccataaccactgagcgactactgtcaggactacgactgtataa
ctctgccacagatcagccagaatgctataagatcacctatccgaaaccattgtactccagta
gcgtaccggcgaactactccgatccacagttcgctgtagctgtctgtaacaactatctgcat
gagaactatccgacagtagcatcttatcagattactgacgagtacgatgcttacttggatat
ggtagacgggacagtcgcctgcctggatactgcaaccttctgccccgctaagcttagaagtt
acccgaaaaaacatgagtatagagccccgaatatccgcagtgcggttccatcagcgatgcag
aacacgctacaaaatgtgctcattgccgcaactaaaagaaattgcaacgtcacgcagatgcg
tgaactgccaacactggactcagcgacattcaatgtcgaatgctttcgaaaatatgcatgta
atgacgagtattgggaggagttcgctcggaagccaattaggattaccactgagtttgtcacc
gcatatgtagctagactgaaaggccctaaggccgccgcactatttgcaaagacgtataattt
ggtcccattgcaagaagtgcctatggatagattcgtcatggacatgaaaagagacgtgaaag
ttacaccaggcacgaaacacacagaagaaagaccgaaagtacaagtgatacaagccgcagaa
cccctggcgactgcttacttatgcgggattcaccgggaattagtgcgtaggcttacggccgt
cttgcttccaaacattcacacgctttttgacatgtcggcggaggattttgatgcaatcatag
cagaacacttcaagcaaggcgacccggtactggagacggatatcgcatcattcgacaaaagc
caagacgacgctatggcgttaaccggtctgatgatcttggaggacctgggtgtggatcaacc
actactcgacttgatcgagtgcgcctttggagaaatatcatccacccatctacctacgggta
ctcgttttaaattcggggcgatgatgaaatccggaatgttcctcacactttttgtcaacaca
gttttgaatgtcgttatcgccagcagagtactagaagagcggcttaaaacgtccagatgtgc
agcgttcattggcgacgacaacatcatacatggagtagtatctgacaaagaaatggctgaga
ggtgcgccacctggctcaacatggaggttaagatcatcgacgcagtcatcggtgagagacca
ccttacttctgcggcggatttatcttgcaagattcggttacttccacagcgtgccgcgtggc
ggatcccctgaaaaggctgtttaagttgggtaaaccgctcccagccgacgacgagcaagacg
aagacagaagacgcgctctgctagatgaaacaaaggcgtggtttagagtaggtataacaggc
actttagcagtggccgtgacgacccggtatgaggtagacaatattacacctgtcctactggc
attgagaacttttgcccagagcaaaagagcattccaagccatcagaggggaaataaagcatc
tctacggtggtcctaaatagtcagcatagtacatttcatctgactaatactacaacaccacc
acctctagaccatggatcccgtcgttttacaacgtcgtgactgggaaaaccctggcgttacc
caacttaatcgccttgcagcacatccccctttcgccagctggcgtaatagcgaagaggcccg
caccgatcgcccttcccaacagttgcgcagcctgaatggcgaatggcgctttgcctggtttc
cggcaccagaagcggtgccggaaagctggctggagtgcgatcttcctgaggccgatactgtc
gtcgtcccctcaaactggcagatgcacggttacgatgcgcccatctacaccaacgtaaccta
tcccattacggtcaatccgccgtttgttcccacggagaatccgacgggttgttactcgctca
catttaatgttgatgaaagctggctacaggaaggccagacgcgaattatttttgatggcgtt
aactcggcgtttcatctgtggtgcaacgggcgctgggtcggttacggccaggacagtcgttt
gccgtctgaatttgacctgagcgcatttttacgcgccggagaaaaccgcctcgcggtgatgg
tgctgcgttggagtgacggcagttatctggaagatcaggatatgtggcggatgagcggcatt
ttccgtgacgtctcgttgctgcataaaccgactacacaaatcagcgatttccatgttgccac
tcgctttaatgatgatttcagccgcgctgtactggaggctgaagttcagatgtgcggcgagt
tgcgtgactacctacgggtaacagtttctttatggcagggtgaaacgcaggtcgccagcggc
accgcgcctttcggcggtgaaattatcgatgagcgtggtggttatgccgatcgcgtcacact
acgtctcaaggtcgaaaacccgaaactgtggagcgccgaaatcccgaatctctatcgtgcgg
tggttgaactgcacaccgccgacggcacgctgattgaagcagaagcctgcgatgtcggtttc
cgcgaggtgcggattgaaaatggtctgctgctgctgaacggcaagccgttgctgattcgagg
cgttaaccgtcacgagcatcatcctctgcatggtcaggtcatggatgagcagacgatggtgc
aggatatcctgctgatgaagcagaacaactttaacgccgtgcgctgttcgcattatccgaac
catccgctgtggtacacgctgtgcgaccgctacggcctgtatgtggtggatgaagccaatat
tgaaacccacggcatggtgccaatcaatcgtctgaccgatgatccgcgctggctaccggcga
tgagcgaacgcgtaacgcgaatggtgcagcgcgatcgtaatcacccgagtgtgatcatctgg
tcgctggggaatgaatcaggccacggcgctaatcacgacgcgctgtatcgctggatcaaatc
tgtcgatccttcccgcccggtgcagtatgaaggcggcggagccgacaccacggccaccgata
ttatttgcccgatgtacgcgcgcgtggatgaagaccagcccttcccggctgtgccgaaatgg
tccatcaaaaaatggctttcgctacctggagagacgcgcccgctgatcctttgcgaatacgc
ccacgcgatgggtaacagtcttggcggtttcgctaaatactggcaggcgtttcgtcagtatc
cccgtttacagggcggcttcgtctgggactgggtggatcagtcgctgattaaatatgatgaa
aacggcaacccgtggtcggcttacggcggtgattttggcgatacgccgaacgatcgccagtt
ctgtatgaacggtctggtctttgccgaccgcacgccgcatccagcgctgacggaagcaaaac
accagcagcagtttttccagttccgtttatccgggcaaaccatcgaagtgaccagcgaatac
ctgttccgtcatagcgataacgagctcctgcactggatggtggcgctggatggtaagccgct
ggcaagcggtgaagtgcctctggatgtcgctccacaaggtaaacagttgattgaactgcctg
aactaccgcagccggagagcgccgggcaactctggctcacagtacgcgtagtgcaaccgaac
gcgaccgcatggtcagaagccgggcacatcagcgcctggcagcagtggcgtctggcggaaaa
cctcagtgtgacgctccccgccgcgtcccacgccatcccgcatctgaccaccagcgaaatgg
atttttgcatcgagctgggtaataagcgttggcaatttaaccgccagtcaggctttctttca
cagatgtggattggcgataaaaaacaactgctgacgccgctgcgcgatcagttcacccgtgc
accgctggataacgacattggcgtaagtgaagcgacccgcattgaccctaacgcctgggtcg
aacgctggaaggcggcgggccattaccaggccgaagcagcgttgttgcagtgcacggcagat
acacttgctgatgcggtgctgattacgaccggtcacgcgtggcagcatcaggggaaaacctt
atttatcagccggaaaacctaccggattgatggtagtggtcaaatggcgattaccgttgatg
ttgaagtggcgagcgatacaccgcatccggcgcggattggcctgaactgccagctggcgcag
gtagcagagcgggtaaactgqctcggattagggccgcaagaaaactatcccgaccgccttac
tgccgcctgttttgaccgctgggatctgccattgtcagacatgtataccccgtacgtcttcc
cgagcgaaaacggtctgcgctgcgggacgcgcgaattgaattatggcccacaccagtggcgc
ggcgacttccagttcaacatcagccgctacagtcaacagcaactgatggaaaccagccatcg
ccatctgctgcacgcggaagaaggcacatggctgaatatcgacggtttccatatggggattg
gtggcgacgactcctggagcccgtcagtatcggcggaattcagctgagcgccgttcgctacc
attaccagttggtctggtgtcaaaaataataataaccgggcaggggggatcctagacgctac
gccccaatgatccgaccagcaaaactcgatgtacttccgaggaactgatgtgcataatgcag
gaattcgatatcaagctagcatgcaggccttgggcccaatgatccgaccagcaaaactcgat
gtacttccgaggaactgatgtgcataatgcatcaggctggtacattagatccccgcttaccg
cgggcaatatagcaacactaaaaactcgatgtacttccgaggaagcgcagtgcataatgctg
cgcagtgttgccacataaccactatattaaccatttatctagcggacgccaaaaactcaatg
tatttctgaggaagcgtggtgcataatgccacgcagcgtctgcataacttttattatttctt
ttattaatcaacaaaattttgtttttaacatttcaaaaaaaaaaaaaaaaaaaaaaaaaaaa
aaaaaaaaagggaattcctcgattaattaagcggccg

### Example Five

### Utility of Recombinant L. monocytogenes (Lm) Strains Expressing Holin, Lysin, or Holin and Lysin, for Delivering Messenger RNA (mRNA) to the Cytoplasm of a Mammalian Host Cell

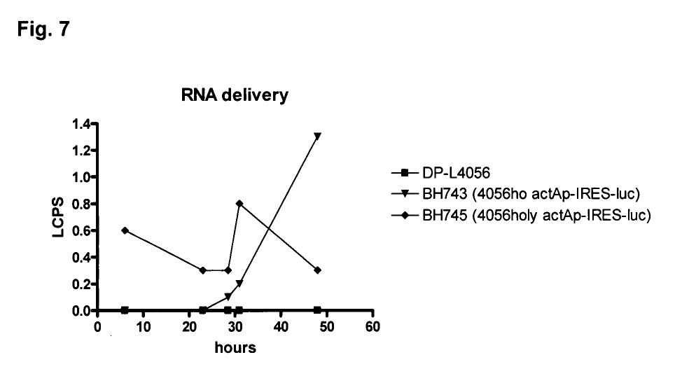

Plasmids encoding an IRES sequence operably linked with a nucleic acid encoding luciferase were transfected into Listeria monocytogenes (Lm), as described below. The Lm, in turn, were used to infect mammalian cells. In the first study (FIG. 7), the plasmid encoded luciferase.

Details of the above work are as follows. For FIG. 7, luminiscence from luciferase activity was a measure of transcription of the RNA in the Lm, release of the RNA to the cytoplasm of the mammalian host cell, and translation of the message. The IRES sequence of the mRNA mediates translation and can substitute, for example, in whole or in part, for a cap sequence on the mRNA. FIG. 7 demonstrates that Lm-holin (BH743) mediated significant release of the mRNA from the bacterium to the mammalian host cell's cytoplasm. Here, luminescence reached about 1.2 LCPS units. Lm-holin-lysin (4056holy) also mediated significant release of the mRNA, where luminescence ranged from 0.3-0.8 LCPS units.

In order to determine the utility of delivering RNA from Listeria to infected host cells, a plasmid was constructed with the luciferase ORF under the control of the actA promoter and containing an intervening IRES sequence. This plasmid allows the transcription of the luciferase message in Listeria, the holin-mediated secretion of the message and subsequent translation in the host cell cytoplasm.

The construction of the plasmid was accomplished by PCR amplifying both a synthetic IRES fragment (ClaI/BamHI ends) and the luciferase ORF from pGL3 control vector (Promega, BamHI/NotI ends). Both PCR products were purified over a Qiagen® column and digested with the appropriate restriction enzymes. The vector pBHE135 was digested with ClaI and NotI, treated with CIP and purified over a Qiagen® column. The vector and two fragments were ligated together using T4 DNA ligase (NEB) in a three-way ligation. Chloramphenicol resistant colonies were screened by colony PCR and confirmed by restriction digest. This plasmid was introduced into Listeria by conjugation (Lauer, et al. (2002) J. Bacteriol. 184:4177-4186), selected on erythromycin and integration confirmed by PCR, resulting in BH721. This strain was cured of the vector backbone sequences (ENGINEERED LISTERIA AND METHODS OF USE THEREOF, U.S. Ser. No. 11/395,197, filed Mar. 30, 2006, and assigned to Cerus Corporation) resulting in erythromycin sensitive colonies (BH741). This strain was subsequently conjugated with SM10 cells harboring either pBHE633 (actAp_holin directed to comK) or pBHE636 (actAp_holin-lysin directed to comK) resulting in the Listeria monocytogenes strains, BH743 and BH745, respectively.

Listeria monocytogenes was introduced into BHK cells as follows. These strains along with DP-L4056 were grown overnight in BHI medium at 30° C. BHK cells were plated at an initial density of 2 E⁵ cells/ml in 12-well plates. The following day 1 ml of bacterial culture was pelleted and resuspended in 1 ml DPBS. This suspension was used to inoculate DMEM+10% FBS+1xNEAA to a final density of 8×10⁷ bacteria/ml.

Medium was removed from BHK cells by aspiration and the cells were inoculated with the bacteria-containing medium (MOI 200). After a 1 hour infection period at 37° C., medium was replaced with DMEM+10% FBS+1xNEAA+50 gentamicin. Cells were assayed for luciferase activity several times over the course of 48 hours as described in Example 3.

### Example Six

### Utility of Recombinant L. monocytogenes (Lm) Strains Expressing Holin, Lysin, or Holin and Lysin for Delivering a Cap-Independent, Viral-Based Replicon to the Cytoplasm of a Mammalian Host Cell

The present invention, in certain aspects, provides a Listeria bacterium encompassing a nucleic acid containing a virus-derived expression cassette, where the expression cassette encodes the following RNA embodiments. In one embodiment, the RNA does not contain an IRES. Alternatively, the expression cassette is derived from a viral genome that does not contain an IRES (e.g., Yellow fever virus; alphavirus), and an IRES is engineered into the RNA.

In another embodiment, the expression cassette is derived from a viral genome that has a naturally occurring IRES (e.g., Picornaviruses), where this IRES is maintained in the expression cassette. In still another aspect, the expression cassette is derived from a viral genome having a naturally occurring IRES, where this IRES is deleted or inactivated. Moreover, what is also supplied is an expression cassette derived from a viral genome having an IRES, but where the IRES is deleted or inactivated and replaced with a different IRES.

The invention includes Listeria, nucleic acids, and methods, where the RNA initially made in the Listeria bacterium is functional in the host cell's cytoplasm, even when the RNA is uncapped. What is also included are Listeria, nucleic acids, and methods, where the RNA is functional in the host cell's cytoplasm, even when the RNA is uncapped, and where subsequent capping increases function, and also where subsequent capping does not increase function.

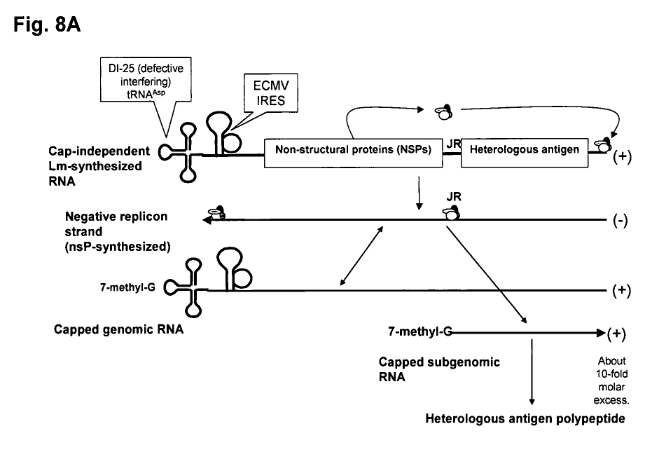

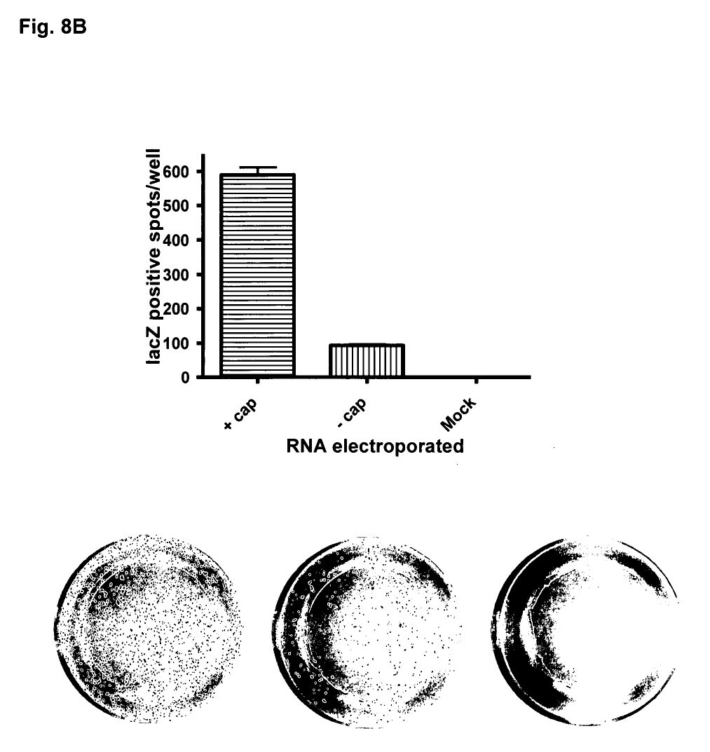

FIG. 8A is a schematic diagram showing replication of the viral-based expression cassette, as it can occur in a mammalian host cell's cytoplasm, as well as biosynthesis of capped subgenomic RNA, where the capped subgenomic RNA is used for expression of a heterologous polypeptide, for example, a tumor antigen or infectious agent antigen.

In one aspect of the present invention, recombinant L. monocytogenes strains expressing holin, lysin, or holin and lysin that in addition encode a viral-based self-amplifying RNA, also known as a replicon, are provided. In such embodiments, the viral-based replicon can be encoded by a plasmid DNA or, alternatively, can be integrated into the bacterial chromosome at any preferred site by non-limiting alternative methods including homologous recombination or by site-specific integration vectors. Replicons derived from viruses having single-stranded RNA genomes of positive polarity, that are encoded by the recombinant L. monocytogenes strains expressing holin, lysin, or holin and lysin are provided. In one embodiment, the replicon is a component of a eukaryotic expression cassette that is functionally linked to a DNA polymerase II (pol II) promoter, including as non-limiting examples the cytomegalovirus (CMV) immediate early or Rous sarcoma virus (RSV) promoters, and encoded by a plasmid DNA. As a non-limiting example, the replicon can be derived from any member of the Togaviridae family, including Sindbis virus (SIN), or Venezuelan equine encephalitis virus (VEE). In this embodiment, the SIN or VEE replicons are deleted of most of their genomes encoding the viral structural proteins (sPs), rendering these expresssed RNA molecules incapable of producing productively infectious virus. While replicons derived from SIN are described, in can be appreciated by those skilled in the art that such embodiments can be derived from any member of the Togaviridae family. As a non-limiting example, replicon compositions containing a heterologous gene of interest, such as a gene encoding a desired tumor antigen, in place of the structural proteins (sPs) are provided. In the described invention, the plasmid encoding the replicon is released into the cytoplasm of infected mammalian host cells in culture or in an intact vaccinated animal following infection with a recombinant L. monocytogenes strain expressing holin, lysin, or holin and lysin. Within this context, following migration to the nucleus and synthesis of the replicon RNA initiated from the pol II promoter, the replicon is transported to the cytoplasm, where the nonstructural proteins (nsPs), or replicase, are first translated by the host cell machinery, which in turn program the amplification of the RNA replicon, by ordered steps including first a synthesis of a full-length replicon-complementary strand of negative polarity, which in turn serves as template for the synthesis of additional replicon full-length RNA molecules and also for the synthesis of subgenomic RNA molecules, through initiation from an internal promoter that is functional only when the RNA is of negative polarity. The subgenomic RNA is the translational template for the encoded gene of interest, and is synthesized in molar excess as compared to the level of full-length RNA replicon molecules synthesized.

In some embodiments of the invention, replicons that are derived from cap-independent viruses having single-stranded RNA genomes of positive polarity, or from viruses having single-stranded RNA genomes of positive polarity that are further modified such that they are cap-independent, are described. Such native or modified virus derived replicons are cap-independent by virtue of containing a functional internal ribosomal entry site (IRES). Such cap-independent replicons can be encoded by a plasmid DNA or, alternatively, can be integrated into the bacterial chromosome of recombinant L. monocytogenes strains that in addition express holin, lysin, or holin and lysin. In this aspect of the invention, the cap-independent replicon RNA is functionally linked to a bacterial promoter, as a non-limiting example a PrfA-inducible promoter such as actA. Synthesis of the cap-independent replicon RNA by the bacterium is induced in the cytoplasm of infected mammalian host cells in culture or in an intact vaccinated animal following infection with a recombinant L. monocytogenes strain, and is released into the mammalian host cell by expression of holin, lysin, or holin and lysin. Subsequently, the nsPs are translated from the cap-independent replicon RNA by the host cell machinery, resulting in self-amplification of the replicon and protein synthesis of the encoded heterologous gene, as described above.

### A. Construction of Cap-Independent Alphavirus Replicons.

Construction of Cap-Independent Sindbis Virus Replicon (pCO390):

A DNA fragment containing the following ordered elements was synthesized by DNA2.0 (Menlo Park, Calif.): a sp6 promoter, sindbis virus tRNA^{Asp} defective-interferring (DI) 5′terminus, UTR and codons 1-40 of nsp1 followed by the internal ribosomal entry site (IRES) from ECMV and codons 1-40 of nsp1 with alternate codon useage and received on the plasmid pJ10:4934. The insert from pJ10:4394 was fused with the Sindbis virus replicon from the plasmid Sinrep/lacZ using SOE-PCR. Briefly, the insert from pJ10:4394 was amplified with the primers:

PL860

(SEQ ID NO: 30)

5′ ATGGAAAAACGCCAGCAACGCGAGCTCGTATGGACATATTGTCGT
TAGAACG 3′
PL861

(SEQ ID NO: 31)

5′ CCTCCAGCTCGATTAGTTTACTGGCCAGGTGGCTGAAGGCTCT
TG 3′

Using the plasmid Sinrep/LacZ as template, a portion of the Sinbis virus replicon was amplified with the following primers:

PL862

(SEQ ID NO: 32)

5′ GAGCCTTCAGCCACCTGGCCAGTAAACTAATCGAGCTGGAGGTT
CC 3′
WL224

(SEQ ID NO: 33)

5′ ATACCGGCCGTGGCTAGTATC 3′

Products from these two primary reactions were pooled and used as template for a secondary amplification with the primer set PL860/WL224. Phusion polymerase® (New England Biolabs, Beverly, Mass.) was used in all amplifications. The secondary PCR product was digested with Sac I and Eag I and inserted into the Sac I and Eag I sites of pBluescript KS+® (Stratagene) and designated pCO330. The fidelity of the insert was verified by sequence analysis. The remainder of the Sindbis virus replicon and lacZ reporter was isolated from a partial Eag I digestion of the plasmid Sinrep/LacZ. Following gel purification, the 8055 bp Eag I fragment was inserted into the Eag I site of pCO330 resulting in the plasmid pCO390.

Shown below is the DNA sequence of the Sindbis virus replicon from plasmid pCO390. The plasmid contains an Sp6 promoter, tRNA end, nsp1 codons 1-40, IRES, nsp1 (1-40) wobble codons, the rest of nsp1-4, lacZ, through EagI restriction site.

(SEQ ID NO: 34)
atttaggggacactatagggatatagtggtgagtatccccgcctgtcacgcgggagaccggg
gttcggttccccgacggggagccaaacagccgaccaattgcactaccatcacaatggagaag
ccagtagtaaacgtagacgtagacccccagagtccgtttgtcgtgcaactgcaaaaaagctt
cccgcaatttgaggtagtagcacagcaggtcactccaaatgaccatgctaatgccagagcat
tttcgcatctggcgcatgcatctagggcggccaattccgcccctctccctccccccccccta
acgttactggccgaagccgcttggaataaggccggtgtgcgtttgtctatatgtgattttcc
accatattgccgtcttttggcaatgtgagggcccggaaacctggccctgtcttcttgacgag
cattcctaggggtctttcccctctcgccaaaggaatgcaaggtctgttgaatgtcgtgaagg
aagcagttcctctggaagcttcttgaagacaaacaacgtctgtagcgaccctttgcaggcag
cggaaccccccacctggcgacaggtgcctctgcggccaaaagccacgtgtataagatacacc
tgcaaaggcggcacaaccccagtgccacgttgtgagttggatagttgtggaaagagtcaaat
ggctctcctcaagcgtattcaacaaggggctgaaggatgcccagaaggtaccccattgtatg
ggatctgatctggggcctcggtgcacatgctttacatgtgtttagtcgaggttaaaaaaacg
tctaggccccccgaaccacggggacgtggttttcctttgaaaaacacgatgataagcttatg
gaaaaaccggtggtcaatgtggatgtcgatccacaaagcccattcgtagtacagcttcagaa
gtcatttccacagttcgaagtggtcgcccagcaagtaaccccgaacgaccacgccaacgcaa
gagccttcagccacctggccagtaaactaatcgagctggaggttcctaccacagcgacgatc
ttggacataggcagcgcaccggctcgtagaatgttttccgagcaccagtatcattgtgtctg
ccccatgcgtagtccagaagacccggaccgcatgatgaaatacgccagtaaactggcggaaa
aagcgtgcaagattacaaacaagaacttgcatgagaagattaaggatctccggaccgtactt
gatacgccggatgctgaaacaccatcgctctgctttcacaacgatgttacctgcaacatgcg
tgccgaatattccgtcatgcaggacgtgtatatcaacgctcccggaactatctatcatcagg
ctatgaaaggcgtgcggaccctgtactggattggcttcgacaccacccagttcatgttctcg
gctatggcaggttcgtaccctgcgtacaacaccaactgggccgacgagaaagtccttgaagc
gcgtaacatcggactttgcagcacaaagctgagtgaaggtaggacaggaaaattgtcgataa
tgaggaagaaggagttgaagcccgggtcgcgggtttatttctccgtaggatcgacactttat
ccagaacacagagccagcttgcagagctggcatcttccatcggtgttccacttgaatggaaa
gcagtcgtacacttgccgctgtgatacagtggtgagttgcgaaggctacgtagtgaagaaaa
tcaccatcagtcccgggatcacgggagaaaccgtgggatacgcggttacacacaatagcgag
ggcttcttgctatgcaaagttactgacacagtaaaaggagaacgggtatcgttccctgtgtg
cacgtacatcccggccaccatatgcgatcagatgactggtataatggccacggatatatcac
ctgacgatgcacaaaaacttctggttgggctcaaccagcgaattgtcattaacggtaggact
aacaggaacaccaacaccatgcaaaattaccttctgccgatcatagcacaagggttcagcaa
atgggctaaggagcgcaaggatgatcttgataacgagaaaatgctgggtactagagaacgca
agcttacgtatggctgcttgtgggcgtttcgcactaagaaagtacattcgttttatcgccca
cctggaacgcagacctgcgtaaaagtcccagcctcttttagcgcttttcccatgtcgtccgt
atggacgacctctttgcccatgtcgctgaggcagaaattgaaactggcattgcaaccaaaga
aggaggaaaaactgctgcaggtctcggaggaattagtcatggaggccaaggctgcttttgag
gatgctcaggaggaagccagagcggagaagctccgagaagcacttccaccattagtggcaga
caaaggcatcgaggcagccgcagaagttgtctgcgaagtggaggggctccaggcggacatcg
gagcagcattagttgaaaccccgcgcggtcacgtaaggataatacctcaagcaaatgaccgt
atgatcggacagtatatcgttgtctcgccaaactctgtgctgaagaatgccaaactcgcacc
agcgcacccgctagcagatcaggttaagatcataacacactccggaagatcaggaaggtacg
cggtcgaaccatacgacgctaaagtactgatgccagcaggaggtgccgtaccatggccagaa
ttcctagcactgagtgagagcgccacgttagtgtacaacgaaagagagtttgtgaaccgcaa
actataccacattgccatgcatggccccgccaagaatacagaagaggagcagtacaaggtta
caaaggcagagcttgcagaaacagagtacgtgtttgacgtggacaagaagcgttgcgttaag
aaggaagaagcctcaggtctggtcctctcgggagaactgaccaaccctccctatcatgagct
agctctggagggactgaagacccgacctgcggtcccgtacaaggtcgaaacaataggagtga
taggcacaccggggtcgggcaagtcagctattatcaagtcaactgtcacggcacgagatctt
gttaccagcggaaagaaagaaaattgtcgcgaaattgaggccgacgtgctaagactgagggg
tatgcagattacgtcgaagacagtagattcggttatgctcaacggatgccacaaagccgtag
aagtgctgtacgttgacgaagcgttcgcgtgccacgcaggagcactacttgccttgattgct
atcgtcaggccccgcaagaaggtagtactatgcggagaccccatgcaatgcggattcttcaa
catgatgcaactaaaggtacatttcaatcaccctgaaaaagacatatgcaccaagacattct
acaagtatatctcccggcgttgcacacagccagttacagctattgtatcgacactgcattac
gatggaaagatgaaaaccacgaacccgtgcaagaagaacattgaaatcgatattacaggggc
cacaaagccgaagccaggggatatcatcctgacatgtttccgcgggtgggttaagcaattgc
aaatcgactatcccggacatgaagtaatgacagccgcggcctcacaagggctaaccagaaaa
ggagtgtatgccgtccggcaaaaagtcaatgaaaacccactgtacgcgatcacatcagagca
tgtgaacgtgttgctcacccgcactgaggacaggctagtgtggaaaaccttgcagggcgacc
catggattaagcagcccactaacatacctaaaggaaactttcaggctactatagaggactgg
gaagctgaacacaagggaataattgctgcaataaacagccccactccccgtgccaatccgtt
cagctgcaagaccaacgtttgctgggcgaaagcattggaaccgatactagccacggccggta
tcgtacttaccggttgccagtggagcgaactgttcccacagtttgcggatgacaaaccacat
tcggccatttacgccttagacgtaatttgcattaagtttttcggcatggacttgacaagcgg
actgttttctaaacagagcatcccactaacgtaccatcccgccgattcagcgaggccggtag
ctcattgggacaacagcccaggaacccgcaagtatgggtacgatcacgccattgccgccgaa
ctctcccgtagatttccggtgttccagctagctgggaagggcacacaacttgatttgcagac
ggggagaaccagagttatctctgcacagcataacctggtcccggtgaaccgcaatcttcctc
acgccttagtccccgagtacaaggagaagcaacccggcccggtcaaaaaattcttgaaccag
ttcaaacaccactcagtacttgtggtatcagaggaaaaaattgaagctccccgtaagagaat
cgaatggatcgccccgattggcatagccggtgcagataagaactacaacctggctttcgggt
ttccgccgcaggcacggtacgacctggtgttcatcaacattggaactaaatacagaaaccac
cactttcagcagtgcgaagaccatgcggcgaccttaaaaaccctttcgcgttcggccctgaa
ttgccttaacccaggaggcaccctcgtggtgaagtcctatggctacgccgaccgcaacagtg
aggacgtagtcaccgctcttgccagaaagtttgtcagggtgtctgcagcgagaccagattgt
gtctcaagcaatacagaaatgtacctgattttccgacaactagacaacagccgtacacggca
attcaccccgcaccatctgaattgcgtgatttcgtccgtgtatgagggtacaagagatggag
ttggagccgcgccgtcataccgcaccaaaagggagaatattgctgactgtcaagaggaagca
gttgtcaacgcagccaatccgctgggtagaccaggcgaaggagtctgccgtgccatctataa
acgttggccgaccagttttaccgattcagccacggagacaggcaccgcaagaatgactgtgt
gcctaggaaagaaagtgatccacgcggtcggccctgatttccggaagcacccagaagcagaa
gccttgaaattgctacaaaacgcctaccatgcagtggcagacttagtaaatgaacataacat
caagtctgtcgccattccactgctatctacaggcatttacgcagccggaaaagaccgccttg
aagtatcacttaactgcttgacaaccgcgctagacagaactgacgcggacgtaaccatctat
tgcctggataagaagtggaaggaaagaatcgacgcggcactccaacttaaggagtctgtaac
agagctgaaggatgaagatatggagatcgacgatgagttagtatggattcatccagacagtt
gcttgaagggaagaaagggattcagtactacaaaaggaaaattgtattcgtacttcgaaggc
accaaattccatcaagcagcaaaagacatggcggagataaaggtcctgttccctaatgacca
ggaaagtaatgaacaactgtgtgcctacatattgggtgagaccatggaagcaatccgcgaaa
agtgcccggtcgaccataacccgtcgtctagcccgcccaaaacgttgccgtgcctttgcatg
tatgccatgacgccagaaagggtccacagacttagaagcaataacgtcaaagaagttacagt
atgctcctccaccccccttcctaagcacaaaattaagaatgttcagaaggttcagtgcacga
aagtagtcctgtttaatccgcacactcccgcattcgttcccgcccgtaagtacatagaagtg
ccagaacagcctaccgctcctcctgcacaggccgaggaggcccccgaagttgtagcgacacc
gtcaccatctacagctgataacacctcgcttgatgtcacagacatctcactggatatggatg
acagtagcgaaggctcacttttttcgagctttagcggatcggacaactctattactagtatg
gacagttggtcgtcaggacctagttcactagagatagtagaccgaaggcaggtggtggtggc
tgacgttcatgccgtccaagagcctgcccctattccaccgccaaggctaaagaagatggccc
gcctggcagcggcaagaaaagagcccactccaccggcaagcaatagctctgagtccctccac
ctctcttttggtggggtatccatgtccctcggatcaattttcgacggagagacggcccgcca
ggcagcggtacaacccctggcaacaggccccacggatgtgcctatgtctttcggatcgtttt
ccgacggagagattgatgagctgagccgcagagtaactgagtccgaacccgtcctgtttgga
tcatttgaaccgggcgaagtgaactcaattatatcgtcccgatcagccgtatcttttccact
acgcaagcagagacgtagacgcaggagcaggaggactgaatactgactaaccggggtaggtg
ggtacatattttcgacggacacaggccctgggcacttgcaaaagaagtccgttctgcagaac
cagcttacagaaccgaccttggagcgcaatgtcctggaaagaattcatgccccggtgctcga
cacgtcgaaagaggaacaactcaaactcaggtaccagatgatgcccaccgaagccaacaaaa
gtaggtaccagtctcgtaaagtagaaaatcagaaagccataaccactgagcgactactgtca
ggactacgactgtataactctgccacagatcagccagaatgctataagatcacctatccgaa
accattgtactccagtagcgtaccggcgaactactccgatccacagttcgctgtagctgtct
gtaacaactatctgcatgagaactatccgacagtagcatcttatcagattactgacgagtac
gatgcttacttggatatggtagacgggacagtcgcctgcctggatactgcaaccttctgccc
cgctaagcttagaagttacccgaaaaaacatgagtatagagccccgaatatccgcagtgcgg
ttccatcagcgatgcagaacacgctacaaaatgtgctcattgccgcaactaaaagaaattgc
aacgtcacgcagatgcgtgaactgccaacactggactcagcgacattcaatgtcgaatgctt
tcgaaaatatgcatgtaatgacgagtattgggaggagttcgctcggaagccaattaggatta
ccactgagtttgtcaccgcatatgtagctagactgaaaggccctaaggccgccgcactattt
gcaaagacgtataatttggtcccattgcaagaagtgcctatggatagattcgtcatggacat
gaaaagagacgtgaaagttacaccaggcacgaaacacacagaagaaagaccgaaagtacaag
tgatacaagccgcagaacccctggcgactgcttacttatgcgggattcaccgggaattagtg
cgtaggcttacggccgtcttgcttccaaacattcacacgctttttgacatgtcggcggagga
ttttgatgcaatcatagcagaacacttcaagcaaggcgacccggtactggagacggatatcg
catcattcgacaaaagccaagacgacgctatggcgttaaccggtctgatgatcttggaggac
ctgggtgtggatcaaccactactcgacttgatcgagtgcgcctttggagaaatatcatccac
ccatctacctacgggtactcgttttaaattcggggcgatgatgaaatccggaatgttcctca
cactttttgtcaacacagttttgaatgtcgttatcgccagcagagtactagaagagcggctt
aaaacgtccagatgtgcagcgttcattggcgacgacaacatcatacatggagtagtatctga
caaagaaatggctgagaggtgcgccacctggctcaacatggaggttaagatcatcgacgcag
tcatcggtgagagaccaccttacttctgcggcggatttatcttgcaagattcggttacttcc
acagcgtgccgcgtggcggatcccctgaaaaggctgtttaagttgggtaaaccgctcccagc
cgacgacgagcaagacgaagacagaagacgcgctctgctagatgaaacaaaggcgtggttta
gagtaggtataacaggcactttagcagtggccgtgacgacccggtatgaggtagacaatatt
acacctgtcctactggcattgagaacttttgcccagagcaaaagagcattccaagccatcag
aggggaaataaagcatctctacggtggtcctaaatagtcagcatagtacatttcatctgact
aatactacaacaccaccacctctagaccatggatcccgtcgttttacaacgtcgtgactggg
aaaaccctggcgttacccaacttaatcgccttgcagcacatccccctttcgccagctggcgt
aatagcgaagaggcccgcaccgatcgcccttcccaacagttgcgcagcctgaatggcgaatg
gcgctttgcctggtttccggcaccagaagcggtgccggaaagctggctggagtgcgatcttc
ctgaggccgatactgtcgtcgtcccctcaaactggcagatgcacggttacgatgcgcccatc
tacaccaacgtaacctatcccattacggtcaatccgccgtttgttcccacggagaatccgac
gggttgttactcgctcacatttaatgttgatgaaagctggctacaggaaggccagacgcgaa
ttatttttgatggcgttaactcggcgtttcatctgtggtgcaacgggcgctgggtcggttac
ggccaggacagtcgtttgccgtctgaatttgacctgagcgcatttttacgcgccggagaaaa
ccgcctcgcggtgatggtgctgcgttggagtgacggcagttatctggaagatcaggatatgt
ggcggatgagcggcattttccgtgacgtctcgttgctgcataaaccgactacacaaatcagc
gatttccatgttgccactcgctttaatgatgatttcagccgcgctgtactggaggctgaagt
tcagatgtgcggcgagttgcgtgactacctacgggtaacagtttctttatggcagggtgaaa
cgcaggtcgccagcggcaccgcgcctttcggcggtgaaattatcgatgagcgtggtggttat
gccgatcgcgtcacactacgtctcaaggtcgaaaacccgaaactgtggagcgccgaaatccc
gaatctctatcgtgcggtggttgaactgcacaccgccgacggcacgctgattgaagcagaag
cctgcgatgtcggtttccgcgaggtgcggattgaaaatggtctgctgctgctgaacggcaag
ccgttgctgattcgaggcgttaaccgtcacgagcatcatcctctgcatggtcaggtcatgga
tgagcagacgatggtgcaggatatcctgctgatgaagcagaacaactttaacgccgtgcgct
gttcgcattatccgaaccatccgctgtggtacacgctgtgcgaccgctacggcctgtatgtg
gtggatgaagccaatattgaaacccacggcatggtgccaatcaatcgtctgaccgatgatcc
gcgctggctaccggcgatgagcgaacgcgtaacgcgaatggtgcagcgcgatcgtaatcacc
cgagtgtgatcatctggtcgctggggaatgaatcaggccacggcgctaatcacgacgcgctg
tatcgctggatcaaatctgtcgatccttcccgcccggtgcagtatgaaggcggcggagccga
caccacggccaccgatattatttgcccgatgtacgcgcgcgtggatgaagaccagcccttcc
cggctgtgccgaaatggtccatcaaaaaatggctttcgctacctggagagacgcgcccgctg
atcctttgcgaatacgcccacgcgatgggtaacagtcttggcggtttcgctaaatactggca
ggcgtttcgtcagtatccccgtttacagggcggcttcgtctgggactgggtggatcagtcgc
tgattaaatatgatgaaaacggcaacccgtggtcggcttacggcggtgattttggcgatacg
ccgaacgatcgccagttctgtatgaacggtctggtctttgccgaccgcacgccgcatccagc
gctgacggaagcaaaacaccagcagcagtttttccagttccgtttatccgggcaaaccatcg
aagtgaccagcgaatacctgttccgtcatagcgataacgagctcctgcactggatggtggcg
ctggatggtaagccgctggcaagcggtgaagtgcctctggatgtcgctccacaaggtaaaca
gttgattgaactgcctgaactaccgcagccggagagcgccgggcaactctggctcacagtac
gcgtagtgcaaccgaacgcgaccgcatggtcagaagccgggcacatcagcgcctggcagcag
tggcgtctggcggaaaacctcagtgtgacgctccccgccgcgtcccacgccatcccgcatct
gaccaccagcgaaatggatttttgcatcgagctgggtaataagcgttggcaatttaaccgcc
agtcaggctttctttcacagatgtggattggcgataaaaaacaactgctgacgccgctgcgc
gatcagttcacccgtgcaccgctggataacgacattggcgtaagtgaagcgacccgcattga
ccctaacgcctgggtcgaacgctggaaggcggcgggccattaccaggccgaagcagcgttgt
tgcagtgcacggcagatacacttgctgatgcggtgctgattacgaccggtcacgcgtggcag
catcaggggaaaaccttatttatcagccggaaaacctaccggattgatggtagtggtcaaat
ggcgattaccgttgatgttgaagtggcgagcgatacaccgcatccggcgcggattggcctga
actgccagctggcgcaggtagcagagcgggtaaactggctcggattagggccgcaagaaaac
tatcccgaccgccttactgccgcctgttttgaccgctgggatctgccattgtcagacatgta
taccccgtacgtcttcccgagcgaaaacggtctgcgctgcgggacgcgcgaattgaattatg
gcccacaccagtggcgcggcgacttccagttcaacatcagccgctacagtcaacagcaactg
atggaaaccagccatcgccatctgctgcacgcggaagaaggcacatggctgaatatcgacgg
tttccatatggggattggtggcgacgactcctggagcccgtcagtatcggcggaattcagct
gagcgccgttcgctaccattaccagttggtctggtgtcaaaaataataataaccgggcaggg
gggatcctagacgctacgccccaatgatccgaccagcaaaactcgatgtacttccgaggaac
tgatgtgcataatgcaggaattcgatatcaagctagcatgcaggccttgggcccaatgatcc
gaccagcaaaactcgatgtacttccgaggaactgatgtgcataatgcatcaggctggtacat
tagatccccgcttaccgcgggcaatatagcaacactaaaaactcgatgtacttccgaggaag
cgcagtgcataatgctgcgcagtgttgccacataaccactatattaaccatttatctagcgg
acgccaaaaactcaatgtatttctgaggaagcgtggtgcataatgccacgcagcgtctgcat
aacttttattatttcttttattaatcaacaaaattttgtttttaacatttcaaaaaaaaaaa
aaaaaaaaaaaaaaaaaaaaaaaaaagggaattcctcgattaattaagcggccgc

To test cap-independent launch of Sindbis virus replicons, the plasmids pCO390 and Sinrep/lacZ were linearized with Not I and Pac I, respectively, and transcribed in vitro using the SP6 Message Machine kit® (Ambion, Inc., Austin, Tex.). Transcription reactions were performed according to the supplied manual except 10 mM NTP solution (Invitrogen Corp., Carlsbad, Calif.) was substituted for the 2× NTP mix provided in the kit. Following transcription, template DNA was removed by DNase digestion and uncapped RNA was purified using the MEGAClear purification kit® (Ambion, Inc., Austin, Tex.) according to supplied instructions. The resulting uncapped message RNA was introduced into BHK cells by electroporation.

For electroporation, late log-phase BHK cells cultured in complete growth medium were trypsinized, washed four times in RNase-free PBS (Ambion, Inc.) and resuspended in RNase-free PBS at 5×10⁷ cell/ml. Immediately prior to electroporation, 20 ug RNA in PBS was mixed with 0.5 mL cell suspension and transferred to a chilled electroporation cuvette (0.4 cm gap). The cells were pulsed twice with 1.65 kV, 25 uF capacitance and infinite resistance. Pulsed cells were incubated 10 minutes at room temperature then suspended in 10 mL complete growth medium and plated on a 96-well plate with 100 uL/well. Electroporated cells were incubated overnight at 37° C. then stained for β-galactosidase activity as described previously.

FIG. 8B discloses a study addressed only the issue of the influence of capping on expression from an mRNA encoding β-galactosidase, where the mRNA contained an IRES sequence operably linked with the nucleic acid encoding β-galactosidase (FIG. 8B). DI-IRES RNA was electroporated into BHK cells and assayed for lacZ positive cells after 24 hr.

The FIG. 8B study involved only mRNA electroporated into BHK cells. Listeria was not used in the second study, that is, L. monocytogens was not used as an intermediary vector. The second study demonstrates expression from the IRES-containing mRNA (capped), and lower expression from the IRES-containing mRNA that is not capped. The figure demonstrates that the viral-derived expression vector (replicon) is functional, even without a cap. The source of the IRES was ECMV (encephalomyocarditis virus). Capped and uncapped RNA was prepared using SP6 Message Machine kit® (Ambion, Inc., Austin, Tex.), where uncapped RNA was made by leaving out the cap analogue from the incubation mixture. Again, the results demonstrate that capping is not a requirement for expression of the DI-IRES RNA.

To test the ability of L. monocytogenes to deliver Cap-independent replicon RNA to infected eukaryotic cells, L. monocytogenes strains are derived that contain a Sindbis virus replicon downstream of an intracellular inducible bacterial promoter. As a non-limiting example, expression cassettes consisting of the actA promoter and Cap-independent Sindbis virus replicons are constructed in the integration vector pINT for stable integration adjacent to the tRNA^{Arg} locus of the Listeria chromosome. The actA promoter is fused to the Cap-independent Sindbis virus replicon from pCO390 by SOE-PCR. The actA promoter including the transcriptional start site are amplified from the plasmid p221 with the primers:

BamHI-PactA

(SEQ ID NO: 35)

5′ GGATCCGGGAAGCAGTTGGGGTTAACTG 3′
PactA DI/IRES REV

(SEQ ID NO: 36)

5′ CGGGGATACTCACCACTATATCCTTATACTCCCTCCTCGTGATAC
GC 3′

The 5′ end of the Sindbis virus replicon is amplified from pCO390 with the following primers:

PactA DI/IRES FOR

(SEQ ID NO: 37)

5′ GCGTATCACGAGGAGGGAGTATAAGGATATAGTGGTGAGTATCCC
CG 3′
WL224

(SEQ ID NO: 33)

5′ ATACCGGCCGTGGCTAGTATC 3′

The products of the above amplifications are pooled and used as template in a subsequent amplification with the primer set BamHI-actA/WL224. The resulting 4169 bp product is digested with BamHI and EagI (sites are underlined in respective primers) and cloned into the BamHI-EagI sites of pINT. The remainder of the sinrep replicon, including the lacZ reporter, is isolated from a partial EagI digest of pCO390 and inserted into the EagI site of the pINT intermediate described above. The pINT-derived plasmids are integrated into the chromosome of designated L. monocytogenes strains following conjugation with an E. coli donor strain and selection on erythromycin. Once integration is confirmed by PCR, pINT vector sequence including the erythromycin resistance marker is excised by transient expression of Cre recombinase. The resulting erythromycin-sensitive strain is then conjugated with an SM10 donor strain containing a second integration vector that inserts at the comK locus of the L. monocytogenes chromosome. This second integration vector includes an expression cassette with holin, lysin or holin and lysin downstream of an inducible promoter for intracellular expression of holin or holin and lysin.

Additional configurations of the Sindbis virus replicon integrated into the genome of L. monocytogenes encoding holin, or holing and lysin to facilitate launch of the Sindbis virus derived replicon RNA in the cytoplasm of infected cells include incorporation of the wild-type 5′end of the Sindbis virus replicon (without the DI tRNA-Asp or ECMV-IRES) or the DI tRNA-Asp alone (without ECMV-IRES) at the 5′end of the replicon.

### B. Construction of Cap-Independent Poliovirus Replicons.

In some embodiments, it is preferred to derive replicons from cap-independent viruses having single-stranded RNA genomes of positive polarity. As a non-limiting example, polioviruses, which belong to the Picornaviridae family, are cap-independent by virtue of an IRES element at the 5′ proximal end of the viral RNA genome, are provided. While replicons derived from polioviruses are described, in can be appreciated by those skilled in the art that such embodiments can be derived from any member of the Picornaviridae family. The poliovirus replicon cDNA was derived by a first RT-PCR step using viral RNA as template that is isolated from a stock of poliovirus Sabin Type 2 human vaccine strain (ATCC VR-301), using the Trizol reagent (Invitrogen, Carlsbad, Calif.). The poliovirus RNA was amplified in two fragments, comprised of a first fragment corresponding to the viral 5′ end to the carboxyl terminus of VP4 (nts. 1-954 according to NCBI accession X00595), and a second fragment corresponding to the amino terminus of the cysteine proteinase 2A viral protein to the viral 3′ end (nts. 3364-7439). Thus, the replicon includes the viral 5′ and 3′ sequences required in cis for replication, the nonstructural proteins which together comprise the viral replicase and proteinase activities for processing of the viral polyprotein, and VP4 as a leader sequence to facilitate efficient translation of a heterologous sequence fused in frame. However, the replicon is deleted of the poliovirus VP2, VP3, and VP1 viral genes and is unable to synthesize productively infectious virus. In some embodiments, the poliovirus replicon was further modified to include a 2A cysteine proteinase recognition amino acid sequence at the junction between the VP4-heterologous antigen fusion protein, as described previously (Porter, et al. (1995) J. Virol. 69:1548-1555). In some embodiments, the poliovirus replicon was still further modified to include two unique restriction endonuclease recognition sites in tandem between the 2A cysteine proteinase recognition amino acid sequence and the amino terminus of the cysteine proteinase 2A viral protein, to facilitate insertion of a sequence encoding a desired protein, as a non-limiting example a tumor antigen or an antigen related to a designated infectious disease, such as hepatitis C virus or influenza. In some embodiments, it is preferred that the sequences of the unique restriction endonuclease recognition sites are configured such that to the translational reading frame of the replicon is maintained following insertion of a desired protein.

As a non-limiting example, the poliovirus (PV) replicon cDNA was inserted into the p217 pINT integration vector (patent ref), for insertion into the listerial chromosome, adjacent to the tRNA^{Arg} gene (Lauer, et al., supra).

The first step of the construction was to insert a prokaryotic promoter sequence, as a non-limiting example, the L. monocytogenes PrfA-inducible actA core promoter, into the p217 vector. The actA core promoter was amplified with Pfx polymerase (Invitrogen, Carlsbad, Calif.) using the primer set WL289/WL290. The resulting PCR product was purified with the MERmaid kit (Bio 101), digested with Kpn I and Sal I and inserted into the multiple cloning sequence of the p217 pINT integration vector between the Kpn I and Xho I sites. This plasmid is known as pIN548. The +1 transcription initiation sequence of the actA core promoter is shown as a bolded A nucleotide base below in the WL290 reverse primer and corresponds to the authentic 5′ end of the polio virus RNA.

Primers for amplification of the actA core promoter sequence with flanking unique 5′ end Kpn I and 3′ Sal I sites (underlined) and “buffer” sequence (lower case) to facilitate restriction enzyme digestion efficiency:

WL289:

(SEQ ID NO: 38)

5′ tatatGGTACCGGGAAGCAGTTGGGGTTAACTG 3′
WL290:

(SEQ ID NO: 39)

5′ atataGTCGACATTTTAAGAATATCACTTGGAGAATTAATTTTTC
TC 3′

As a non-limiting example for the derivation of the PV-derived cap-independent replicon cDNA, the first fragment corresponding to the viral 5′ end to the carboxyl terminus of VP4 (nts. 1-954) was generated by RT-PCR using the primers described below:

First strand cDNA synthesis:

| WL294 (reverse; viral nts. 1020-999): | |
|---|---|
| 5′ CGTTGAATTGCCCAGAGTTAGC 3′ | (SEQ ID NO: 40) |

First strand cDNA synthesis was accomplished with the AffinityScript First-strand Synthesis System® (Stratagene, San Diego, Calif.) according to the manufacturer's specifications, using approximately 200 ng of purified PV genomic RNA. The cDNA product was used directly to amplify the 5′ end PV replicon cDNA fragment by a standard PCR protocol, using the primer set shown below:

PCR amplification primer set:
WL297 (forward; viral nts. 1-23, underlined):

(SEQ ID NO: 41)

5′ atat-GTCGAC-TTAAAACAGCTCTGGGGTTGTAC 3′

In addition to complementarity with PV nts. 1-23 (underlined), beginning at its 5′ end, primer WL297 contains “buffer” sequence (lowercase) to facilitate restriction endonuclease digestion and a Sal I site. While the ordered elements are shown separated by dashes to facilitate identification of each motif, the primer is synthesized as a single 33 base-long nucleic acid.

WL295 [reverse; viral nts. 954-927 (underlined)]:

(SEQ ID NO: 42)

5′ atta-CGGCCG-
TCCATATGTGTCGAGCAGTTTTTG-GTTTAGCATGGGAGCGGTCTTAATA
AGG 3′

In addition to complementarity with PV nts. 954-927 (underlined), beginning at its 5′ end, primer WL295 contains sequences corresponding to the following ordered elements: “buffer” sequence to facilitate restriction endonuclease digestion, Eag I site, and reverse complementary sequence corresponding to the authentic PV 3D polymerase 2A cysteine protease cleavage site (QKLLDTYG, SEQ ID NO:43). While the ordered elements are shown separated by dashes to facilitate identification of each motif, the primer is synthesized as a single 62 base-long nucleic acid.

The resulting amplicon product generated from the PCR reaction with the WL297/WL295 primer set was purified over a Qiagen® column, digested with Sal I and Eag I, and inserted into the multiple cloning sequence of the pIN548 integration vector plasmid between the unique Sal I and Eag I sites. This plasmid will be known as pIN586. The sequence of the WL297/WL295 amplicon is shown below.

WL297/WL295 amplicon sequence:

(SEQ ID NO: 44)

ATATGTCGACTTAAAACAGCTCTGGGGTTGTACCCACCCCAGAGGCCCAC
GTGGCGGCTAGCACTCCGGTATTACGGTACCCTTGTGCGCCTGTTTTATA
CTCCCCTCCCGCAACTTAGAAGCACGAAACCAAGTTCAATAGAAGGGGGT
ACAAACCAGTACCACTACGAACAAGCACTTCTGTTTCCCCGGTGACATTG
CATAGACTGCTCACGCGGTTGAAAGTGATCGATCCGTTACCCGCTTGTGT
ACTTCGAAAAGCCTAGTATCGCCTTGGAATCTTCGACGCGTTGCGCTCAG
CACCCGACCCCGGGGTGTAGCTTAGGCTGATGAGTCTGGACATTCCTCAC
CGGTGACGGTGGTCCAGGCTGCGTTGGCGGCCTACCTATGGCTAACGCCA
TAGGACGTTAGATGTGAACAAGGTGTGAAGAGCCTATTGAGCTACATAAG
AGTCCTCCGGCCCCTGAATGCGGCTAATCCTAACCACGGAACAGGCGGTC
GCGAACCAGTGACTGGCTTGTCGTAACGCGCAAGTCTGTGGCGGAACCGA
CTACTTTGGGTGTCCGTGTTTCCTGTTATTTTTATCATGGCTGCTTATGG
TGACAATCAGAGATTGTTATCATAAAGCGAATTGGATTGGCCATCCGGTG
AGTGTTGTGTCAGGTATACAACTGTTTGTTGGAACCACTGTGTTAGCTTT
ACTTCTCATTTAACCAATTAATCAAAAACAATACGAGGATAAAACAACAA
TACTACAATGGGCGCCCAAGTTTCATCACAGAAAGTTGGAGCCCACGAAA
ATTCAAACAGAGCCTATGGCGGGTCCACCATCAATTACACTACAATCAAT
TACTATAGGGACTCTGCAAGCAATGCAGCAAGCAAGCAAGATTTTGCACA
AGATCCGTCCAAGTTCACCGAACCCATTAAGGACGTCCTTATTAAGACCG
CTCCCATGCTAAACCAAAAACTGCTCGACACATATGGACGGCCGTAAT

As a non-limiting example for the derivation of the poliovirus-derived cap-independent replicon cDNA, the second fragment corresponding to the amino terminus of the cysteine proteinase 2A viral protein to the viral 3′ end (nts. 3386-7440) was generated by RT-PCR using the primers described below:

First strand cDNA synthesis:
WL282 (reverse; viral nts. 5067-5055, underlined):

(SEQ ID NO: 45)

5′ TTTTTTTTTTTTTTTTTTTTTTTTTTTTTTTTTTTTTTTT-CTCCGA
ATTAAAG 3′

In addition to complementarity with PV nts. 7439-7427 (underlined), beginning at its 5′ end, primer WL282 also preferably contains 40 consecutive T residues that are complementary with the PV 3′ end polyadenylation sequence, or poly(A) tail. While the ordered elements are shown separated by dashes to facilitate identification of each motif, the primer is synthesized as a single 53 base-long nucleic acid.

First strand cDNA synthesis was accomplished with the AffinityScript First-strand Synthesis System® (Stratagene, San Diego, Calif.) according to the manufacturer's specifications, using approximately 200 ng of purified PV genomic RNA. The cDNA product was used directly to amplify the 5′ end PV replicon cDNA fragment by a standard PCR protocol, using the primer set shown below:

PCR amplification primer set:
WL296 (forward; viral nts. 3364-3392, underlined):

(SEQ ID NO: 46)

5′ atta-CGGCCGTTTAAACCCTGCAGG-GAAAAGGGATTAACGACTTA
TGGATTTGG 3′

In addition to PV nts. 3364-3392 (underlined), beginning at its 5′ end, primer WL296 contains sequences corresponding to the following ordered elements: buffer sequence to facilitate endonuclease digestion, overlapping EagI and PmeI sites and an Sbf I site. While the ordered elements are shown separated by dashes to facilitate identification of each motif, the primer is synthesized as a single 54 base-long nucleic acid.

WL298 (reverse; viral nts. 5067-5055, underlined):

(SEQ ID NO: 47)

5′ gcgc-TTAATTAA-TTTTTTTTTTTTTTTTTTTTTTTTTTTTTTTTT
TTTTTTT-CTCCGAATTAAAG 3′

In addition to complementarity with PV nts. 7439-7427 (underlined), beginning at its 5′ end, primer WL298 also preferably contains buffer sequence to facilitate endonuclease digestion (lower case), a Pac I site, followed by 40 consecutive T residues that are complementary with the PV 3′ end polyadenylation sequence. While the ordered elements are shown separated by dashes to facilitate identification of each motif, the primer is synthesized as a single 65 base-long nucleic acid.

The resulting amplicon product generated from the PCR reaction with the WL296/WL298 primer set was purified over a Qiagen® column, digested with Eag I and Pac I, and then cloned into the the multiple cloning sequence of the pIN586 integration vector plasmid between the unique EagI and Pad sites. This plasmid is known as pIN599. The sequence of the WL296/WL298 amplicon is shown below.

WL296/WL298 amplicon sequence:

(SEQ ID NO: 48)
ATTACGGCCGTTTAAACCCTGCAGGGAAAAGGGATTAACGACTTATGGATTTGGACACCAAA
ACAAAGCTGTGTACACAGCTGGCTACAAAATTTGCAATTACCACCTAGCTACACAAGAAGAC
TTGCAAAATGCCGTGAGTGTCATGTGGAACAGAGACCTCTTAGTGGCTGAATCAAGGGCCCT
TGGCACCGACTCGATCGCAAGGTGCAGCTGTAACACGGGTGTGTACTACTGTGAATCCAGGA
GAAAATATTATCCAGTTTCTTTCATTGGGCCCACCTTCCAATACATGGAAGCCAATGAATAT
TACCCGGCTAGATATCAATCACACATGCTTATTGGTCATGGGTTTGCATCACCGGGTGATTG
TGGTGGCATACTTAGATGTCAACACGGGGTGATAGGAATAATCACTGCTGGTGGGGAAGGCT
TGGTTGCATTTTCAGACATTAGAGACCTGTATGCTTATGAGGAGGAAGCTATGGAGCAGGGC
ATTTCCAACTATATTGAGTCACTTGGTGCTGCATTTGGTAGTGGATTCACTCAACAAATTGG
TGATAAAGTTTCCGAGCTAACCAGCATGGTAACTAGCACCATTACAGAGAAGTTGCTTAAAA
ACTTAATCAAAATTATCTCATCACTTGTGATCATTACCAGGAATTATGAGGACACTACCACA
GTGCTTGCCACCCTCGCCCTCCTTGGGTGCGACATCTCACCGTGGCAGTGGCTAAAGAAGAA
GGCATGTGACATCCTGGAAATTCCATACGCCATCAAACAAGGAGATAGTTGGTTGAAGAAAT
TCACTGAGGCATGTAATGCTGCAAAGGGACTGGAGTGGGTGTCCAATAAGATATCCAAATTC
ATTAGTTGGTTGCAGGATAAAATCATCCCACAAGCGAGAGACAAATTAGAGTTTGTCACTAA
ACTAAAGCAATTAGAAATGCTTGAAAATCAGATTTCCACCATACACCAATCTTGTCCAAGTC
AAGAACATCAGGAGATCTTATTCAACAATGTGCGGTGGCTATCTATCCAGTCCAAGAGGTTT
GCACCACTATATGCACATGAAGCTAAAAGGATTCAAAAGCTGGAGCATACCATAAATAATTA
CGTACAGTTCAAGAGCAAGCACCGTATTGAGCCAGTATGTTTGTTAGTACATGGCAGTCCAG
GGACAGGAAAATCAGTTGCAACCAATCTAATTGCTAGAGCAATAGCCGAGAAAGAGAACACC
TCCACATACTCACTGCCACCTGATCCGTCTCACTTTGATGGCTACAAGCAACAGGGTGTGGT
TATTATGGATGACCTAAACCAAAATCCAGACGGAGCAGACATGAAACTTTTTTGTCAAATGG
TGTCCACTGTGGAGTTTATTCCACCGATGGCCTCGCTAGAAGAGAAAGGCATTTTGTTCACA
TCTAATTACGTTTTAGCCTCCACCAACTCCAGTCGGATCACACCACCCACGGTGGCTCACAG
TGATGCGCTGGCCAGGAGATTCGCATTTGACGTGGACATACAAGTCATGAGCGAGTACTCCA
GAGACGGAAAGCTCAACATGGCAATGGCTACTGAAATGTGCAAAAACTGTCATCAACCAGCA
AACTTCAAAAGATGTTGTCCTTTAGTGTGTGGCAAGGCAATTCAGTTAATGGATAAATCTTC
CAGGGTTAGATACAGCATTGATCAGATCACTACAATGATTGTTAATGAGAGAAACAGAAGAT
CAAACATTGGTAATTGCATGGAAGCTCTATTCCAGGGACCACTGCAGTATAAAGATCTAAAA
ATAGATGTTAAGACCAGTCCCCCTCCGGAGTGTATCAACGATTTGCTCCAGGCAGTTGATTC
CCAGGAAGTGAGAGATTACTGTGAAAAGAAAGGCTGGATTGTTAACATTACCAGTCAGGTTC
AAACAGAGAGGAACATCAACCGGGCGATGACTATCCTACAAGCAGTAACTACTTTCGCTGCA
GTAGCCGGTGTCGTGTACGTTATGTACAAGCTGTTCGCTGGGCACCAGGGTGCATACACTGG
TTTGCCAAATAAACGACCCAATGTACCCACTATCAGGACAGCAAAAGTGCAAGGCCCTGGGT
TTGATTACGCAGTGGCCATGGCTAAAAGAAACATTGTTACAGCAACCACCAGCAAAGGGGAG
TTTACGATGTTGGGAGTCTATGATAATGTGGCCATCTTGCCAACCCACGCCTCACCTGGTGA
AAGCATTGCGATCGACGGTAAAGAGGTGGAAATTCTTGACGCCAAAGCCCTTGAAGATCAGG
CAGGAACTAATCTTGAAATTACCATAATTACACTAAAGAGGAACGAGAAGTTCAGAGATATC
AGGCCACACATTCCCACCCAAATCACCGAAACAAATGATGGAGTTTTGATCGTGAACACTAG
TAAGTACCCCAACATGTATGTTCCCGTTGGTGCTGTGACCGAACAGGGGTATCTTAATCTCG
GTGGACGACAAACCGCTCGTACGCTAATGTACAACTTTCCAACTAGAGCAGGTCAGTGTGGT
GGTGTCATCACGTGCACTGGTAAAGTCATTGGGATGCATGTTGGTGGGAACGGTTCACATGG
GTTCGCGGCGGCCCTAAAGCGGTCATACTTCACTCAGATTCAAGGTGAGATTCAATGGATGA
AACCATCAAAAGAAGTGGGATACCCGATCATAAATGCTCCGTCCAAAACCAAACTTGAACCC
AGCGCTTTTCACTATGTGTTTGAAGGGGTGAAGGAACCAGCAGTCCTTACCAAAAATGATCC
CAGGCTCAGGACAGACTTTGAAGAAGCAATATTCTCTAAGTATGTAGGCAACAAGATCACTG
ATGTGGATGAGTACATGAAAGAGGCAGTGGATCATTACGCTGGCCAACTCATGTCTCTAGAC
ATCAACACAGAACAAATGTGCTTGGAGGACGCCATGTACGGCACCGATGGCCTGGAAGCACT
TGACTTGACCACTAGTGCTGGATACCCTTATGTAGCAATGGGAAAGAAAAAGAGAGACATCT
TGAATAAGCAGACTAGAGACACCAAGGAAATGCGGAGACTCTTAGATACTTATGGAATTAAC
TTACCGCTTGTAACATATGTTAAAGATGAACTAAGGTCAAAAACTAAGGTGGAGCAGGGAAA
ATCCAGATTGATTGAAGCCTCCAGTTTGAATGATTCAGTGGCCATGAGAATGGCATTTGGAA
ATCTCTATGCAGCATTTCACAAAAACCCAGGAGTTGTCACTGGCAGTGCAGTTGGTTGTGAT
CCAGATCTATTTTGGAGCAAGATCCCAGTGCTAATGGAAGAGAAGCTCTTTGCTTTTGACTA
CACAGGTTATGATGCATCACTCAGCCCGGCCTGGTTTGAGGCACTCAAAATGGTGCTAGAGA
AAATCGGATTTGGGGACAGGGTGGATTATATTGATTACCTCAACCATTCCCACCACCTGTAC
AAAAACAAAACTTATTGCGTAAAAGGCGGCATGCCATCTGGCTGCTCAGGCACATCAATTTT
TAACTCAATGATTAACAACTTAATCATTAGGACACTCCTACTGAAAACCTACAAGGGCATAG
ATTTAGATCACCTAAAGATGATTGCCTATGGTGATGATGTAATTGCTTCCTACCCCCATGAG
GTTGATGCTAGTCTCCTAGCCCAATCAGGAAAAGACTATGGACTAACCATGACTCCAGCAGA
CAAGTCAGCTACCTTTGAAACAGTCACATGGGAGAATGTAACATTCTTGAAAAGATTCTTTA
GAGCGGATGAGAAGTATCCCTTCCTCATACATCCAGTAATGCCAATGAAGGAGATTCATGAA
TCAATTAGATGGACAAAGGATCCCAGAAACACACAGGATCACGTGCGCTCATTGTGCCTATT
GGCCTGGCACAACGGCGAAGAAGAATACAACAAGTTCTTAGCTAAAATCAGGAGTGTGCCAA
TTGGGAGAGCTTTATTGCTCCCAGAGTACTCTACATTGTACCGCCGTTGGCTCGACTCTTTT
TAGTAACCCTACCTCAGTCGAATTGGATTGGGTCATGCTGTTGTAGGGGTAAATTTTTCTTT
AATTCGGAGAAAAAAAAAAAAAAAAAAAAAAAAAAAAAAAAAAAAAAAATTAATTAAGCGC

The final step in the construction of the PV replicon cDNA was to functionally link the actA promoter core sequence with the authentic 5′ end of the PV replicon cDNA by splice overlap extension (SOE) PCR. The SOE PCR step removed the Sal I GTCGAC recognition site intervening the actA promoter core and the PV 5′ end of the pIN599 plasmid. Upon deletion of the Sal I site, this plasmid was designated as pIN662. The DNA sequence corresponding to the actA core promoter and polio replicon from pIN662 is shown below.

actA-Pcore-PVrep sequence from pIN662:

(SEQ ID NO: 49)
CGGGAAGCAGTTGGGGTTAACTGATTAACAAATGTTAGAGAAAAATTAATTCTCCAAGTGAT
ATTCTTAAAATTTAAAACAGCTCTGGGGTTGTACCCACCCCAGAGGCCCACGTGGCGGCTAG
CACTCCGGTATTACGGTACCCTTGTGCGCCTGTTTTATACTCCCCTCCCGCAACTTAGAAGC
ACGAAACCAAGTTCAATAGAAGGGGGTACAAACCAGTACCACTACGAACAAGCACTTCTGTT
TCCCCGGTGACATTGCATAGACTGCTCACGCGGTTGAAAGTGATCGATCCGTTACCCGCTTG
TGTACTTCGAAAAGCCTAGTATCGCCTTGGAATCTTCGACGCGTTGCGCTCAGCACCCGACC
CCGGGGTGTAGCTTAGGCTGATGAGTCTGGACATTCCTCACCGGTGACGGTGGTCCAGGCTG
CGTTGGCGGCCTACCTATGGCTAACGCCATAGGACGTTAGATGTGAACAAGGTGTGAAGAGC
CTATTGAGCTACATAAGAGTCCTCCGGCCCCTGAATGCGGCTAATCCTAACCACGGAACAGG
CGGTCGCGAACCAGTGACTGGCTTGTCGTAACGCGCAAGTCTGTGGCGGAACCGACTACTTT
GGGTGTCCGTGTTTCCTGTTATTTTTATCATGGCTGCTTATGGTGACAATCAGAGATTGTTA
TCATAAAGCGAATTGGATTGGCCATCCGGTGAGTGTTGTGTCAGGTATACAACTGTTTGTTG
GAACCACTGTGTTAGCTTTACTTCTCATTTAACCAATTAATCAAAAACAATACGAGGATAAA
ACAACAATACTACAATGGGCGCCCAAGTTTCATCACAGAAAGTTGGAGCCCACGAAAATTCA
AACAGAGCCTATGGCGGGTCCACCATCAATTACACTACAATCAATTACTATAGGGACTCTGC
AAGCAATGCAGCAAGCAAGCAAGATTTTGCACAAGATCCGTCCAAGTTCACCGAACCCATTA
AGGACGTCCTTATTAAGACCGCTCCCATGCTAAACCAAAAACTGCTCGACACATATGGACGG
CCGTTTAAACCCTGCAGGGAAAAGGGATTAACGACTTATGGATTTGGACACCAAAACAAAGC
TGTGTACACAGCTGGCTACAAAATTTGCAATTACCACCTAGCTACACAAGAAGACTTGCAAA
ATGCCGTGAGTGTCATGTGGAACAGAGACCTCTTAGTGGCTGAATCAAGGGCCCTTGGCACC
GACTCGATCGCAAGGTGCAGCTGTAACACGGGTGTGTACTACTGTGAATCCAGGAGAAAATA
TTATCCAGTTTCTTTCATTGGGCCCACCTTCCAATACATGGAAGCCAATGAATATTACCCGG
CTAGATATCAATCACACATGCTTATTGGTCATGGGTTTGCATCACCGGGTGATTGTGGTGGC
ATACTTAGATGTCAACACGGGGTGATAGGAATAATCACTGCTGGTGGGGAAGGCTTGGTTGC
ATTTTCAGACATTAGAGACCTGTATGCTTATGAGGAGGAAGCTATGGAGCAGGGCATTTCCA
ACTATATTGAGTCACTTGGTGCTGCATTTGGTAGTGGATTCACTCAACAAATTGGTGATAAA
GTTTCCGAGCTAACCAGCATGGTAACTAGCACCATTACAGAGAAGTTGCTTAAAAACTTAAT
CAAAATTATCTCATCACTTGTGATCATTACCAGGAATTATGAGGACACTACCACAGTGCTTG
CCACCCTCGCCCTCCTTGGGTGCGACATCTCACCGTGGCAGTGGCTAAAGAAGAAGGCATGT
GACATCCTGGAAATTCCATACGCCATCAAACAAGGAGATAGTTGGTTGAAGAAATTCACTGA
GGCATGTAATGCTGCAAAGGGACTGGAGTGGGTGTCCAATAAGATATCCAAATTCATTAGTT
GGTTGCAGGATAAAATCATCCCACAAGCGAGAGACAAATTAGAGTTTGTCACTAAACTAAAG
CAATTAGAAATGCTTGAAAATCAGATTTCCACCATACACCAATCTTGTCCAAGTCAAGAACA
TCAGGAGATCTTATTCAACAATGTGCGGTGGCTATCTATCCAGTCCAAGAGGTTTGCACCAC
TATATGCACATGAAGCTAAAAGGATTCAAAAGCTGGAGCATACCATAAATAATTACGTACAG
TTCAAGAGCAAGCACCGTATTGAGCCAGTATGTTTGTTAGTACATGGCAGTCCAGGGACAGG
AAAATCAGTTGCAACCAATCTAATTGCTAGAGCAATAGCCGAGAAAGAGAACACCTCCACAT
ACTCACTGCCACCTGATCCGTCTCACTTTGATGGCTACAAGCAACAGGGTGTGGTTATTATG
GATGACCTAAACCAAAATCCAGACGGAGCAGACATGAAACTTTTTTGTCAAATGGTGTCCAC
TGTGGAGTTTATTCCACCGATGGCCTCGCTAGAAGAGAAAGGCATTTTGTTCACATCTAATT
ACGTTTTAGCCTCCACCAACTCCAGTCGGATCACACCACCCACGGTGGCTCACAGTGATGCG
CTGGCCAGGAGATTCGCATTTGACGTGGACATACAAGTCATGAGCGAGTACTCCAGAGACGG
AAAGCTCAACATGGCAATGGCTACTGAAATGTGCAAAAACTGTCATCAACCAGCAAACTTCA
AAAGATGTTGTCCTTTAGTGTGTGGCAAGGCAATTCAGTTAATGGATAAATCTTCCAGGGTT
AGATACAGCATTGATCAGATCACTACAATGATTGTTAATGAGAGAAACAGAAGATCAAACAT
TGGTAATTGCATGGAAGCTCTATTCCAGGGACCACTGCAGTATAAAGATCTAAAAATAGATG
TTAAGACCAGTCCCCCTCCGGAGTGTATCAACGATTTGCTCCAGGCAGTTGATTCCCAGGAA
GTGAGAGATTACTGTGAAAAGAAAGGCTGGATTGTTAACATTACCAGTCAGGTTCAAACAGA
GAGGAACATCAACCGGGCGATGACTATCCTACAAGCAGTAACTACTTTCGCTGCAGTAGCCG
GTGTCGTGTACGTTATGTACAAGCTGTTCGCTGGGCACCAGGGTGCATACACTGGTTTGCCA
AATAAACGACCCAATGTACCCACTATCAGGACAGCAAAAGTGCAAGGCCCTGGGTTTGATTA
CGCAGTGGCCATGGCTAAAAGAAACATTGTTACAGCAACCACCAGCAAAGGGGAGTTTACGA
TGTTGGGAGTCTATGATAATGTGGCCATCTTGCCAACCCACGCCTCACCTGGTGAAAGCATT
GCGATCGACGGTAAAGAGGTGGAAATTCTTGACGCCAAAGCCCTTGAAGATCAGGCAGGAAC
TAATCTTGAAATTACCATAATTACACTAAAGAGGAACGAGAAGTTCAGAGATATCAGGCCAC
ACATTCCCACCCAAATCACCGAAACAAATGATGGAGTTTTGATCGTGAACACTAGTAAGTAC
CCCAACATGTATGTTCCCGTTGGTGCTGTGACCGAACAGGGGTATCTTAATCTCGGTGGACG
ACAAACCGCTCGTACGCTAATGTACAACTTTCCAACTAGAGCAGGTCAGTGTGGTGGTGTCA
TCACGTGCACTGGTAAAGTCATTGGGATGCATGTTGGTGGGAACGGTTCACATGGGTTCGCG
GCGGCCCTAAAGCGGTCATACTTCACTCAGATTCAAGGTGAGATTCAATGGATGAAACCATC
AAAAGAAGTGGGATACCCGATCATAAATGCTCCGTCCAAAACCAAACTTGAACCCAGCGCTT
TTCACTATGTGTTTGAAGGGGTGAAGGAACCAGCAGTCCTTACCAAAAATGATCCCAGGCTC
AGGACAGACTTTGAAGAAGCAATATTCTCTAAGTATGTAGGCAACAAGATCACTGATGTGGA
TGAGTACATGAAAGAGGCAGTGGATCATTACGCTGGCCAACTCATGTCTCTAGACATCAACA
CAGAACAAATGTGCTTGGAGGACGCCATGTACGGCACCGATGGCCTGGAAGCACTTGACTTG
ACCACTAGTGCTGGATACCCTTATGTAGCAATGGGAAAGAAAAAGAGAGACATCTTGAATAA
GCAGACTAGAGACACCAAGGAAATGCGGAGACTCTTAGATACTTATGGAATTAACTTACCGC
TTGTAACATATGTTAAAGATGAACTAAGGTCAAAAACTAAGGTGGAGCAGGGAAAATCCAGA
TTGATTGAAGCCTCCAGTTTGAATGATTCAGTGGCCATGAGAATGGCATTTGGAAATCTCTA
TGCAGCATTTCACAAAAACCCAGGAGTTGTCACTGGCAGTGCAGTTGGTTGTGATCCAGATC
TATTTTGGAGCAAGATCCCAGTGCTAATGGAAGAGAAGCTCTTTGCTTTTGACTACACAGGT
TATGATGCATCACTCAGCCCGGCCTGGTTTGAGGCACTCAAAATGGTGCTAGAGAAAATCGG
ATTTGGGGACAGGGTGGATTATATTGATTACCTCAACCATTCCCACCACCTGTACAAAAACA
AAACTTATTGCGTAAAAGGCGGCATGCCATCTGGCTGCTCAGGCACATCAATTTTTAACTCA
ATGATTAACAACTTAATCATTAGGACACTCCTACTGAAAACCTACAAGGGCATAGATTTAGA
TCACCTAAAGATGATTGCCTATGGTGATGATGTAATTGCTTCCTACCCCCATGAGGTTGATG
CTAGTCTCCTAGCCCAATCAGGAAAAGACTATGGACTAACCATGACTCCAGCAGACAAGTCA
GCTACCTTTGAAACAGTCACATGGGAGAATGTAACATTCTTGAAAAGATTCTTTAGAGCGGA
TGAGAAGTATCCCTTCCTCATACATCCAGTAATGCCAATGAAGGAGATTCATGAATCAATTA
GATGGACAAAGGATCCCAGAAACACACAGGATCACGTGCGCTCATTGTGCCTATTGGCCTGG
CACAACGGCGAAGAAGAATACAACAAGTTCTTAGCTAAAATCAGGAGTGTGCCAATTGGGAG
AGCTTTATTGCTCCCAGAGTACTCTACATTGTACCGCCGTTGGCTCGACTCTTTTTAGTAAC
CCTACCTCAGTCGAATTGGATTGGGTCATGCTGTTGTAGGGGTAAATTTTTCTTTAATTCGG
AGAAAAAAAAAAAAAAAAAAAAAAAAAAAAAAAAAAAAAAAATTAATTAA

A heterologous sequence, encoding as non-limiting examples a desired tumor antigen(s) (e.g., Mesothelin), infectious disease antigens including as non-limiting examples hepatitis C virus (e.g., core, NS3, NS5b), human immunodeficiency virus (e.g., gag, env, pol, nef), influenza virus (e.g., hemagglutinin (HA), neuraminidase (NA)), or biochemical reporters (e.g. β-galactosidase), can be inserted between unique Eag I, Pme I and Sbf I sites present between the VP4 and 2A cysteine proteinase encoding sequences of the pIN662 plasmid. In this configuration, the antigen is cleaved from the PV replicon expressed polyprotein by the autocatalytic processing activity of the PV 2A cysteine proteinase. As a non-limiting example, a fusion protein consisting of an ovalbumin epitope SL8 and b-galactosidase was inserted into the EagI site of pIN662 to construct the plasmid pBHE893.

Following insertion of the heterologous sequences into the pIN662 plasmid and confirming the fidelity of the PV replicon cDNA by sequence analysis, the resultling plasmid was integrated at the tRNA^{Arg} locus in the genome of selected L. monocytogenes strains using previously described methods (Lauer, et al. (2002) J. Bacteriol. 184:4177-4186). Integration was confirmed on chloramphenicol resistant L. monocytogenes colonies by PCR with NC16 (5′ GTCAAAACATACGCTCTTATC 3′; SEQ ID NO:50) and PL95 (5′ ACATAATCAGTCCAAAGTAGATGC 3′; SEQ ID NO:51).

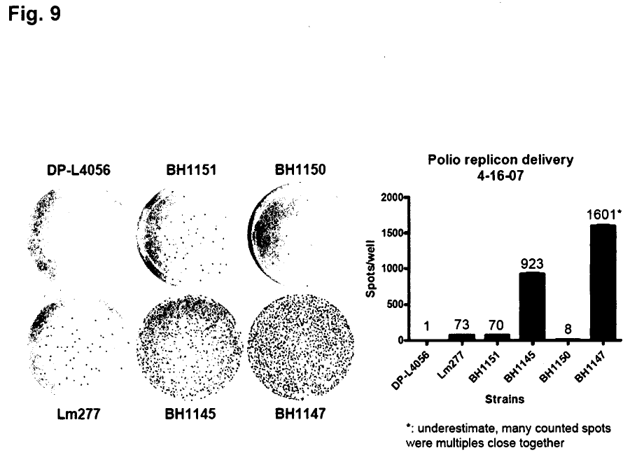

In order to test the ability of L. monocytogenes to deliver a functional polio virus derived (PV) replicon RNA to infected cells, the plasmid pBHE893 was integrated into selected strains of L. monocytogenes, including strains expressing holin and lysin, and the resulting strains were then used to infect BHK cells as described previously. Briefly, BHK cells were seeded at 2×10⁴ cells per well in a white 96-well tissue culture plate with a clear bottom (Optilux, BD Biosciences, Franklin Lake, N.J.). Cells were cultured overnight in complete growth medium without antibiotics. BHK cells were infected at a multiplicity of 200 cfu per cell with fresh overnight cultures of Listeria strains grown at 30° C. in BHI broth. Strains used in this study are listed in Table 9. Infected cells were cultured for 24 hours at 37° C. in complete growth medium containing 50 μg/mL gentamycin to inhibit extracellular growth of Listeria. At 24 hours post-infection, cells were fixed in formaldehyde and stained for β-galactosidase activity as described previously (FIG. 9). Infected cells were imaged and cells positive for b-galactosidase were enumerated using an ImmunoSpot plate reader (CTL, Cleveland, Ohio). FIG. 9 shows that β-galactosidase activity in BHK cells was dependent on infection with L. monocytogenes strains that carry nucleic acids encoding viral replicons. The number of cells positive for β-galactosidase activity was greatly enhanced upon infection with L. monocytogenes strains harboring a PV and also co-expressing both holin and lysin, indicating that L. monocytogenes can deliver nucleic acids to infected cells and that the lytic activity of holin and lysin provide a means by which functional replicon nucleic acids can be released into the cytosol of infected cells (FIG. 9).

TABLE 9
Lm strains for delivering cap-independent viral-based replicon
to the cytoplasm of a mammalian host cell.

| Strain | Background | Holin/lysin | Replicon |
|---|---|---|---|
| DP-L4056 | WT | − | none |
| Lm277 | WT | + (holin only) | Sindbis virus (DNA delivery) |
| BH1151 | WT | − | Polio (RNA delivery) |
| BH1145 | WT | + | Polio (RNA delivery) |
| BH1150 | ΔactAΔinlB | − | Polio (RNA delivery) |
| BH1147 | ΔactAΔinlB | + | Polio (RNA delivery) |

In another embodiment, the PV replicon decribed above was cloned downstream of the full-length actA promoter from L. monocytogenes. In addition to the core sequence sufficient for intracellular inducible expression, the full length actA promoter includes the 5′ untranslated region of the actA transcript, 150 nt in length, that results in enhanced actA expression in the cytoplasm of infected host cells. The PV replicon was cloned downstream of the full length actA promoter to test the transcriptional activity of the full-length actA promoter. The full-length actA promoter was amplified from L. monocytogenes genomic DNA and spliced to the 5′end of the polio replicon in by SOE-PCR. The DNA sequence corresponding to the full-length actA promoter is shown below.

(SEQ ID NO: 52)

gggaagcagttggggttaactgattaacaaatgttagagaaaaattaatt
ctccaagtgatattcttaaaaTaattcatgaatattttttcttatattag
actattaagaagataattaactgctaatccaatttttaacggaataaatt
agtgaaaatgaaggccgaattttccttgttctaaaaaggttgtattagcg
tatcacgaggagggagtataaT

The first bold T is the actA transcription start site, the last bold T is the first nt of the polio replicon

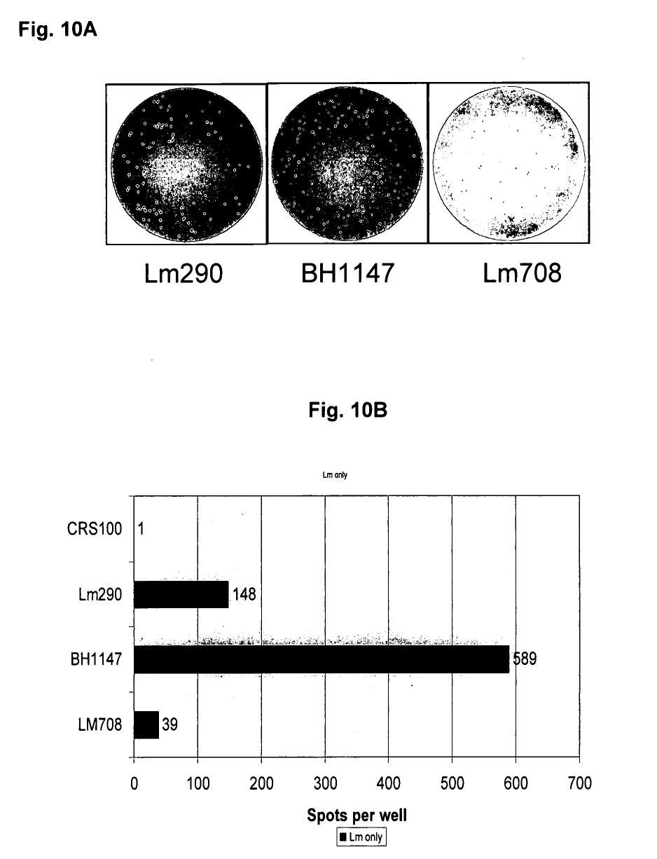

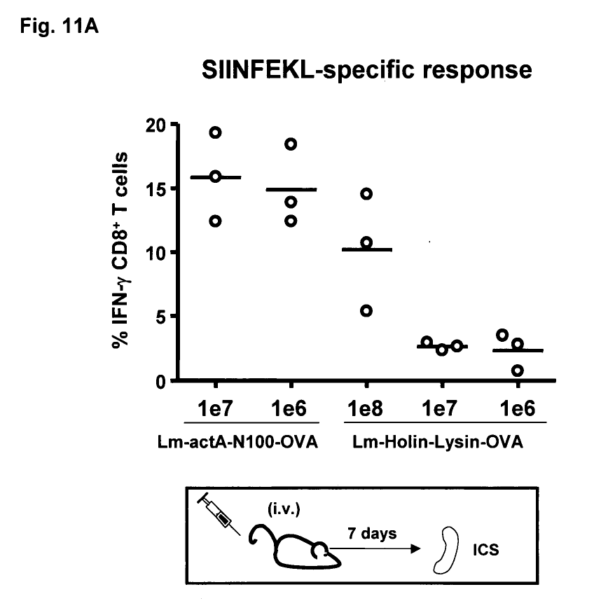

The SOE PCR product was cloned upstream of the poliovirus replicon in pBHE893, replacing the actA core promoter, to construct the plasmid pIN691. The plasmid was then integrated into the genomes of select L. monocytogenes strains expressing holin and lysin to assess how the full-length actA promoter, with the 150 nt untranslated region, impacts the efficiency gene expression from replicons delivered to infected cells. In this embodiment, the PV replicon RNA delivered to infected cells included the 150 nt 5′ untranslated region of the ActA protein that does not constitute the authentic 5′ end of the polio virus. See Table 10 for a description of Listeria strains used in this study. BHK cells cultured in a 96-well plate were infected with the indicated Lm strain at an MOI=300 and stained for β-galactosidase activity at 24 hrs. post-infection. All replicons contained a β-galactosidase reporter (FIG. 10A). Infected cells were photographed and cells stained positive for β-galactosidase were counted using an ImmunoSpot reader (FIG. 10B). As shown in FIG. 10A, when compared to the replicon in which the authentic 5′ end of the polio virus was conserved, the presence of the untranslated region of actA resulted in a greater than 10-fold decrease in the number of infected BHK cells positive for β-galactosidase expression. This data demonstrated that, in some embodiments, maintaining the authentic 5′ terminus of the PV replicon provides optimal expression of a designated antigen(s), including those related to malignant and/or infectious disease.

TABLE 10
Lm strains for delivering a cap-independent viral-based
replicon to the cytoplasm of a mammalian host cell.

| | | | | Replicon |
|---|---|---|---|---|
| Strain | Background | Holin/lysis | Replicon | promoter |
| CRS-100 | ΔactAΔinlB | na | na | na |
| Lm290 | ΔactAΔinlB | + | Sindbis virus | RSV |
| BH1147 | ΔactAΔinlB | + | Polio | actA core |
| Lm708 | ΔactAΔinlB | + | Polio | actA full-length |

L. monocytogenes induces a type I interferon (IFN) response in infected host cells upon phagosomal escape into the host cell cytoplasm. The IFN response arrests host cell protein synthesis, an effect that could diminish replicon-mediated gene expression and self-amplification when launched from L. monocytogene. The host cell interferon response can be inhibited by expression and secretion of one or more viral proteins known to suppress the IFN pathway. These proteins have been shown to reduce the IFN response by diverse mechanisms (see Table 11). As non-limiting examples, a selected protein(s) including those listed in the Table will be expressed from replicon-encoding L. monocytogenes. In some embodiments, such proteins will be expressed and secreted by L. monocytogenes either constituitively or induced upon infection of the host cell. In another embodiment, non-secreted variants of these proteins could be released into the infected host cell cytoplasm by co-expression of holin or holin and lysin.

TABLE 11
Exemplary viral proteins that may be expressed by Listeria to
mitigate effects of type I interferon.

| Protein | Source | Mechanism of Action | Reference |
|---|---|---|---|
| TRBP | human | inhibition of interferon induced protein | Proc Natl Acad Sci USA 1994 May |
| | | kinase PKR | 24; 91(11): 4713-7 |
| Matrix | VSV | Blocks the nuclear pore and prevents | Cancer Cell. 2003. 4: 263-75 |
| (M) | | transport of IFN-β to the cytoplasm | |
| protein | | | |
| L(pro) | FMDV | blocks expression of type-1 IFN protein, | J. Virol. 2006 Feb; 80(4): 1906-14 |
| | | reduces induction of immediate-early | |
| | | induction of IFN-beta mRNA | |
| V | paramyxo | interacts with mda-5 to block activity | Proc Natl Acad Sci USA 2004 Dec |
| protein | viruses | | 7; 101(49): 17264-9 |
| E3L | vaccinia | inhibits ds-RNA-dependent protein | Virology 1998 Apr 10; 243(2): 406-14 |
| | | kinase | |
| B8R | vaccinia | mimics IFN-gamma receptor | J Gen Virol. 2002 Aug; 83(Pt8): 1953-64 |
| NS3 | HCV | protoeolytically cleaves MAVS/IPS-1 | Proc Natl Acad Sci USA 2006 May |
| | | | 30; 103(22): 8499-504 |
| NS1 & | BRSV | inhibits IRF-3 activation | J. Virol. 2003 Aug; 77(16): 8661-8 |
| NS2 | | | |
| N^{pro} | BVDV | blocks type-1 IFN induction | J. Virol. 2006 Jan; 80(2): 900-11 |
| gamma | HSV-1 | insensitivity to alpha-IFN | J. Virol. 2004 Sep; 78(18): 10193-6 |
| (1)34.5/ | | | |
| US11 | | | |
| sigmaA | avian | blocks intracellular enzyme pathways | J. Virol. 2000 Feb; 74(3): 1124-31 |
| | reovirus | dependent on ds-RNA | |
| NS2 | HRSV | suppresses type-1 IFN | J. Virol. 2006 Jun; 80(12): 5958-67 |
| NS1 | influenza | inhibits innate immunity by preventing | J. Virol. 2006 Jul; 80(13): 6295-304 |
| | virus | type-1 IFN release, inhibits adaptive | |
| | | immunity by attenuating DC maturation | |
| UL- | HSV-2 | interferes with IFNa/b-mediated anti- | J. Virol. 2003 Sep; 77(17): 9337-45 |
| 41(vhs) | | viral response | |

An alternative means to address the IFN response entails launching a viral replicon derived from parent viruses with decreased sensitivity to the host cell interferon response. Well known to those who are skilled in the art, parent viruses with INF resistant phenotypes can be selected following serial passages in host cells capable of mounting an INF response and thus inhibiting virus replication and productive growth. Alternatively, INF resistance could be engineered in parent viruses using a reverse genetics approach in which precise mutations are introduced in the viral genome using standard molecular biology techniques. Also, portions of viral genomes from unrelated viruses to which INF-resistance has been mapped can be combined with cap-independent viral replicons to create novel cap-independent, INF-resistant chimeric replicons.

### Example Seven

### Utility of Recombinant L. monocytogenes Strains Expressing Holin, Lysin, or Holin and Lysin, for Eliciting a Specific Immune Response to an Encoded Induced Heterologous Antigen in a Vaccinated Mammal

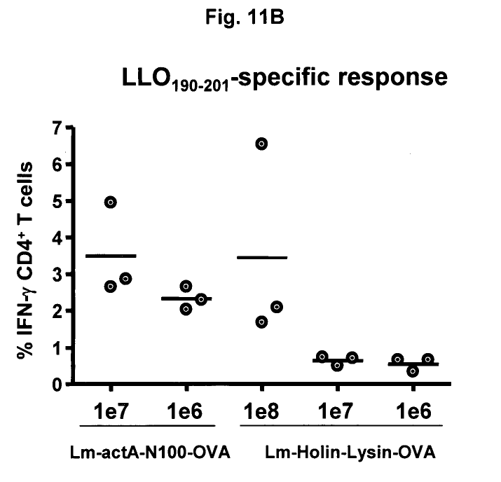

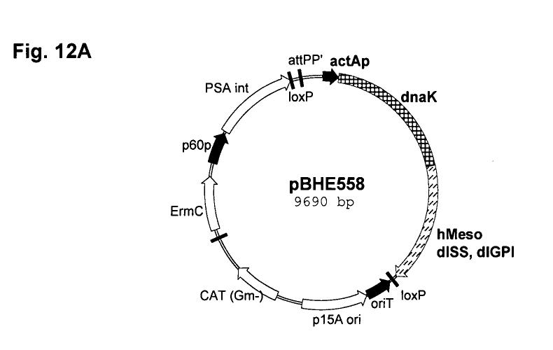

FIGS. 11A and 11B disclose Lm-induced immune response in mice. The Lm constructs were Lm-actA-OVA (no holin; no lysin) and Lm-holin-lysin-OVA (+holin; +lysin). The term “OVA” means that the Lm contained a nucleic acid encoding ovalbumin. The nucleic acid encoding ovalbumin was operably linked and in frame with actA-N 100. Transfer of ovalbumin from the Listeria bacterium to the environment of the host cell's cytosol was mediated by lysis of the bacterium, in the case of Lm-holin-lysin-OVA, and was mediated by a listerial secretory pathway, in the case of Lm-ActA-N100-OVA.

FIG. 11A discloses that administration of both preparations of Lm resulted in ovalbumin specific immune response, where equivalent immune reponses occurred at about 1×10⁶ cfu of Lm-actA-N100-OVA, and 1×10⁸ cfu of Lm-holin-lysin-OVA. SIINFEKL (SEQ ID NO:53), a peptide from ovalbumin, was added to splenocytes harvested from vaccinated mice, where this addition allowing detection of any ovalbumin-specific immune response that had been produced in response to the vaccination.

Concomittant control immune response studies monitored immune response to a secreted protein endogenous to Lm, namely, listeriolysin O (LLO) (FIG. 11B). Listeriolysin expression and secretion was not expected to be influenced by the engineered nucleic acid encoding actA-N100-OVA, or by the engineered nucleic acid encoding holin-lysin-OVA. The results demonstrate that immune response to listeriolysin was equivalent where mice were administered with 1×10⁶ Lm-actA-N100-OVA and 1×10⁸ Lm-holin-lysin-OVA. Lower immune response against listeriolysin was found with administration of the lesser doses of Lm-holin-lysin-OVA.

### Example Eight

### Recombinant Listeria monocytogenes Strains Expressing Holin, Lysin, or Holin and Lysin, where the Heterologous Antigen does not Contain a Bacterial Secretory Peptide

Non-secretory Lm embodiments are provided, where the Lm contains a nucleic acid encoding a heterologous antigen, and where release of the expressed antigen from the Lm is mediated by an expressed holin. In some cases, the antigen may be a macromolecule.

Holin-mediated permeabilization of the listerial membrane can allow transit of an expressed heterologous antigen out of the bacterium and into an external environment, e.g., an external environment that is the cytoplasm of a mammalian host cell.

What is included are embodiments where greater than 10%, greater than 25%, greater than 50%, greater than 75%, and greater than 99%, of the expressed antigen is released. Also, what is included are embodiments where release is greater than 10% dependent on the expressed holin, greater than 25%, greater than 50%, greater than 75%, or greater than 99% dependent on the expressed holin.

In some embodiments, what is provided is Lm containing a nucleic acid encoding a heterologous antigen, and where the nucleic acid does not encode any secretory sequence. Also provided is Lm containing a nucleic acid encoding a heterologous antigen, where the nucleic acid encodes a peptide derived from a secretory sequence, but the secretory sequence is mutated to prevent secretion, or to prevent essentially all secretion. Moreover, in some embodiments, what is encompassed is a Lm containing a polynucleotide comprising a first nucleic acid encoding a heterologous antigen, where the nucleic acid does not encode any secretory sequence, and a second nucleic acid encoding a non-secretory sequence, such as groEL.

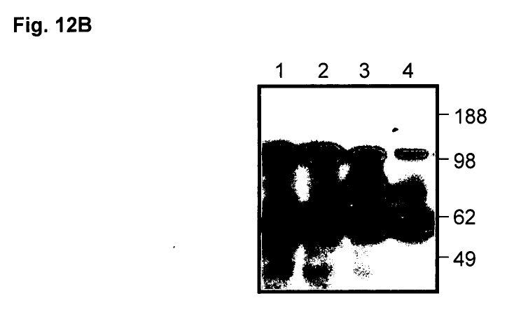

FIGS. 12A and 12B disclose holin-dependent release of a fusion protein from Lm. The fusion protein did not contain any secretory sequence. The bacterium, Lm-holin, had been engineered to express holin. A plasmid encoding the fusion protein, which contained the human Mesothelin polypeptide sequence (FIG. 12A), was transfected into Lm-holin, and the plasmid-containing Lm-holin was used to infect mammalian cells (J774 cells). Once Lm-holin was in the J774 cells, the intracellular environment activated the actA promoter of the plasmid, resulting in production of the fusion protein. The J774 cells were incubated for 7 hours, the cells were disrupted, and soluble protein was analyzed by SDS-PAGE (FIG. 12B).

In short, the activities occurring during incubation of the J774 cells included expression of holin, expression of the fusion protein, and holin-mediated release of the fusion protein from the bacterium to the host cell's cytosol.

As mentioned above, after the incubation, the J774 cells were disrupted, followed by centrifugation to remove insoluble matter (including all bacteria) and separation of soluble proteins by SDS-PAGE. Mesothelin was deterred by the Western blotting method using a polyclonal antibody against Mesothelin.

The experiment was also conducted with control strains of Lm, that is, parental Lm (“CRS-100”) (no plasmid); Lm-holin (no plasmid); and Lm-holin-lysin (plus plasmid).

The following strains of Listeria monocytogenes were used to infect J774 cells. The lanes that are identified below and indicate the lane of the SDS-PAGE gel used to separate the expressed/release fusion protein:
LANE ONE. Lm ΔactAΔinlB (“CRS-100”);
LANE TWO. BH543 (dnaK-hMesothelin in CRS-100);
LANE THREE. BH757 (pBHE588 in CRS-100 plus holin);
LANE FOUR. Bh759 (pBHE588 in CRS-100 plus holin and lysin).

The position of migration of the expressed fusion protein is shown by the arrow. The results of the gel demonstrate no expressed/released fusion protein where the bacteria were Lm ΔactAΔinlB (no plasmid), Lm ΔactAΔinlB (plus plasmid), and very slight expression/release where the bacteria were Lm ΔactAΔinlB-holin-lysin (plus plasmid). In striking contrast, dramatic expression/release occurred with Lm ΔactAΔinlB-holin (plus plasmid).

The results in the gel also show two non-specific bands of staining, residing above and below the Mesothelin band.

The results demonstrate that Lm-holin is an effective agent for expressing and releasing polypeptides into a host cell, even where the polypeptide is not a secretory protein. Without implying any limitation on the present invention, the results also demonstrate that in this instance, Lm-holin-lysin was a relatively poor agent for expressing and releasing the indicated polypeptide into the host cell.

What is provided is Lm-holin, vaccines comprising Lm-holin, and related methods. These methods include using Lm-holin for the intracellular expression and release of any substance, e.g., a water-soluble substance, a macromolecule, a polypeptide, a heterologous antigen, a tumor antigen, an infectious agent antigen, a complex including a polypeptide, a nucleic acid, a plasmid, a viral-based expression cassette, a ssRNA (positive strand) viral-based expression cassette, and the like.

In some embodiments, what is also provided is Lm-holin that does not contain any recombinant nucleic acid encoding a lysin, vaccines comprising Lm-holin that does not contain a recombinant nucleic acid encoding a lysin, and related methods. These methods include using the Lm-holin that does not contain any recombinant nucleic acid encoding a lysin, for the expression and release of any substance, e.g., a water-soluble substance, a macromolecule, a polypeptide, a heterologous antigen, a tumor antigen, an infectious agent antigen, a complex including a polypeptide, a nucleic acid, a plasmid, a viral-based expression cassette, a ssRNA (positive strand) viral-based expression cassette, and the like.

Moreover, in some embodiments, what is provided is a method for stimulating immune response against a heterologous antigen, comprising administering a Lm-holin-heterologous antigen where the stimulated immune response is greater than that obtainable with administering a suitable control Lm that lacks a nucleic acid encoding holin, where the greater is at least 20% greater; 50% greater; 100% greater (2-fold greater); 5-fold greater; 10-fold greater, or more. A suitable control is Lm-heterologous antigen (no holin).

Also, what is encompassed is a method for stimulating immune response against a heterologous antigen, comprising administering a Lm-holin-heterologous antigen (lacking a recombinant nucleic acid encoding a lysin) where the stimulated immune response is greater than that obtainable with administering a suitable control Lm that lacks a nucleic acid encoding holin, where the greater is at least 20% greater; 50% greater; 100% greater (2-fold greater); 5-fold greater; 10-fold greater, or more. A suitable control is Lm-heterologous antigen that lacks a recombinant nucleic acid encoding a lysin (no holin).

The Lm of the invention can contain a more than one nucleic acids, each encoding the same holin, each encoding a different holin, or any combination thereof. The nucleic acids can be operably linked with a promoter specifically activated by an environment found inside a host mammalian cell, e.g., prfA promoter, any prfA-activatable promoter, actA promoter, promoters sensitive to low iron concentrations, and so on. Constitutively active promoters are also available. The promoter can also be one specifically used by a particular RNA polymerase, where this RNA polymerase is expressed (or activated) in increased amounts by the Lm when the Lm is inside a mammalian host cell.

Experimental details were as follows. These details are not intended to limit the invention. In order to test the utility of holin-lysin strains for delivering antigens lacking a bacterial secretory peptide, the following plasmids were engineered. A human mesothelin ORF optmized for expression in Listeria was synthesized (CCN16543, Blue Heron Biotechnology) and used as template for PCR with the following primer set:

PL339 (forward):

(SEQ ID NO: 54)

5′ GGGCGGCCGCGAGCTCTTAGCCTTGTAAACCTAAACCTAATGTATC
TAA 3′
PL340 (reverse):

(SEQ ID NO: 55)

5′ GGGGATCCCGTACATTAGCAGGTGAAACAGGTCAAGAA 3′

The PCR product (huMesothelin Δsignal sequence Δgpi anchor) was purified over a Qiagen® column and digested with BamHI and EagI. The pINT vector containing the actA promoter, pBHE135, was digested with the same set of restriction enzymes, treated with CIP and purified over a Qiagen® column. The vector and insert were ligated together using T4 DNA ligase. Chloramphenicol resistant colonies were screened by PCR and confirmed by restriction digest, resulting in pBHE139.

The groEL ORF was PCR amplified from DP-L4056 genomic DNA using the following primer set:

PL714 (forward):

(SEQ ID NO: 56)

5′ AAAATCGATATGAGCAAAATTATCGGAATTGACTTA 3′
PL715 (reverse):

(SEQ ID NO: 57)

5′ AAAGGATCCTTTGTTTTCTTTGTCGTCGTCATTTAC 3′

The PCR product was purified over a Qiagen® column and digested with ClaI and BamHI. The plasmid pBHE139 was digested with the same set of restriction enzymes, treated with CIP (NEB) and purified over a Qiagen® column. Vector and insert were ligated together using T4 DNA ligase. Chloramphenicol resistant colonies were screened by PCR and positive clones confirmed by restriction digest. This resulted in the plasmid pBHE558. This plasmid was transferred to Listeria strain CRS 100 by conjugation (Lauer et al., supra) resulting in the erythromycin resistant strain BH543. This strain was cured of vector backbone sequences (ENGINEERED LISTERIA AND METHODS OF USE THEREOF, U.S. Ser. No. 11/395,197), resulting in the erythromycin sensitive strain BH755.

To engineer holin and holin-lysin expressing variants of the groEL-huMesothelin strain, BH755 was conjugated with SM10 cells containing either pBHE633 (actAp_holin directed to comK locus) or pBHE636 (actAp_holin-lysin directed to comK locus). The selection of erythromycin resistant colonies resulted in BH757 (holin only) and BH759 (holin-lysin) expressing variants. Levels of expression and secretion of the groEL-huMesothelin protein was analyzed by Western blot in both broth and in mammalian host cells.

### Example Nine

### Recombinant Listeria monocytogenes Strains Expressing Holin, or Holin and Lysin, where the Nucleic Acid Encoding Holin is not from a Listeriophage or from a Listerial Genus

What is provided is a Listeria monocytogenes bacterium containing a nucleic acid encoding a holin that is not listerial and not from a listeriophage. The nucleic acid can be from a bacteriophage that is not a listeriophage, from a bacterium that is not listerial, or from other organisms.

The invention encompasses non-listeriophage holins, including Serratia marcescens NucE (Berkmen, et al. (1997) 179:6522-6524); Staphylococcus aureus bacteriophage 187 holin (Loessner, et al. (1999) J. Bacteriol. 181:4452-4460; phage lambda holins and Lactobacillus gasseri phi-adh holin (Henrich, et al. (1995) J. Bacteriol. 177:723-732; phage phi-29 holin (Steiner, et al. (1993) J. Bacteriol. 175:1038-1042); Bacillus phage PZA holin (Loessner, et al. (1997) J. Bacteriol. 179:2845-2851); phage T4 gpt holin (Dressman and Drake (1999) J. Bacteriol. 181:4391-4396); phage PRD1 holin (Ziedaite, et al. (2005) J. Bacteriol. 187:5397-5405); Borrelia burgdorferi prophage BIyA (Damman, et al. (2000) J. Bacteriol. 182:6791-6797); Bacillus subtilis ywcE holin (Real, et al. (2005) J. Bacteriol. 187:6443-6453); Staphylococcus aureus lrgA holin and cidA holin (Brunskill and Bayles (1996) J. Bacteriol. 178:5810-5812; Rice, et al. (2004) J. Bacteriol. 186:3029-3037); Streptococcus pneumoniae cph1 holin, pneumococcal phage EJ-1 holin, phi-LC3 holin and Tuc2009 holin of Lactococcus lactis phage (Martin, et al. (1998) J. Bacteriol. 180:210-217); bacteriophage P2 gene Y holin (Ziermann, et al. (1994) J. Bacteriol. 176:4974-4984); and bacteriophage PRD1 holin P35 (Rydman and Bamford (2003) J. Bacteriol. 185:3795-3803).

Nucleic acids in bacterial genomes, encoding proteins identified as holins and holin-like proteins, are available (Table 12). Some of these holins can be characterized as bacterial holins, and not as holins of cryptic phages, i.e., phage genomes integrated in the bacterial genome. These holin genes include the CidA gene of S. aureus (see, e.g., Rice, et al. (2003) J. Bacteriol. 185:2635-2643; Rice and Bayles (2003) Mol. Microbiol. 50:729-738; Bayles (2000) Trends Microbiol. 8:274-278; GenBank Acc. No. AY581892).

In some embodiments, what is also contemplated are listerial strains that are not Lm, for example, L. innocua engineered to contain factors that mediate entry into antigen presenting cells, and that mediate exit from the phagolysosome to the host cell's cytoplasm.

TABLE 12
Bacterial genomic nucleic acids encoding holins.

| Bacterium | GenBank Acc. No. |
|---|---|
| Bacillus subtilis | Z99117, nt 51006-51428. |
| Bacillus subtilis | NC_000964, nt 2263876-2264088; 3932232-3932618. |
| Bacillus anthracis strain Sterne | NC_005945, five holins, e.g., 3432919-3433284. |
| Pseudomonas entomophila | NC_008027, compl. nt 4463886-4464239. |
| Escherichia coli | BA000007, ten holins, e.g., nt 901806-902021. |
| Listeria monocytogenes strain | NC_002973 nt 142006-142428. |
| 4b F2365 | |
| Listeria innocua | AL596169, nt compl. 165378-165638. |
| Staphylococcus epidermidis | CP000029, nt 2047024-2047482. |
| Erwinia carotovora | NC_004547, compl. nt 2950159-2950467. |
| Corynebacterium diphtheriae | NC_002935, six holins, e.g., nt 3637616-3637981. |
| Corynebacterium diphtheriae | BX248360, compl. nt 129273-129650. |
| Staphylococcus aureus | AJ938182, four holins, e.g., compl. 1846356-1846793. |
| Salmonella typhimurium | AE008823 nt 16310-16529. |
| Rhodopseudomonas palustris | BX572594, nt 258068-258451. |

### Example Ten

### Tables Identifying Exemplary strains of Listeria monocytogenes and Plasmid Constructs

TABLE 13A
Plasmid Constructions

| | |
|---|---|
| pSH263 | pSH252 with Sindbis virus/lacZ replicon |
| | downstream of RSV promoter |
| pSH252 | pAM401 with oriT for transfer from E. coli donor to |
| | Listeria by conjugation. |
| Sinrep/lacZ | Sindbis virus replicon with lacZ reporter |
| Sinrep21 | Sinbis virus replicon with RSV promoter for DNA |
| | launch |
| pINT (aka p217) | Integration vector and source of oriT |
| pAM401 | E. coli/L. monocytogenes shuttle plasmid |
| pSH258 | pSH252 with BamHI-EagI fragment from Sinrep21 |
| pBHE530 | pAM-CMV-lacZ |
| pJ10:4934 | Sindbis virus DI/IRES 5′end |
| pCO330 | Sinrep DI/IRES intermediate |
| pCO390 | Sinrep DI/IRES replicon |
| pIN548 | pINT containing actA core promoter |
| pIN586 | PIN548 with 5′ end of polio replicon including |
| | IRES, VP4, 2A cleavage site |
| pIN599 | pIN586 with 3′ end of polio replicon including |
| | nonstructural viral proteins and poly A tail |
| pIN662 | pIN599 with complete polio replicon functionally |
| | linked to actA core promoter. |
| pBHE893 | pIN662 with SL8-b galactosidase fusion |

Bacteria Strains

| | |
|---|---|
| SM10 | E. coli conjugation donor strain |
| B-Ec-266 | SM10 containing pSH263 |
| 4029uvr | |
| DP-L4056 | |
| BH334 | 4056 holin |
| BH336 | 4056 lysin |
| BH276 | 4056 holin-lysin |
| B-Lm-274 | 4056 containing pSH263 |
| B-Lm-276 | BH336 with pSH263 |
| B-Lm-277 | BH334 with pSH263 |
| B-Lm-278 | BH276 with pSH263 |
| BH276(pBHE530) | BH276 with pBHE530 |
| BH727 | 4056 with two copies of holin |
| B-Lm-284 | 4029uvr::pBHE292, 4029uvr holin-lysin |
| B-Lm-288 | B-Lm-284 with pSH263 |
| B-Lm-289 | 4029uvr with pSH263 |
| B-Lm-414 | BH727 with pSH263 |

TABLE 13B
Strains of Listeria monocytogenes.

| | | | Site of integration | |
|---|---|---|---|---|
| Strain | Derived from | Plasmid | or episomal | Features |
| ActAN100_OVA(AH1A5) | CRS-100 | none | n/a | Contains N- |
| | | | | terminal fusion |
| | | | | of first 100 aa of |
| | | | | ActA to |
| | | | | OVA(AH1A5) |
| | | | | under the control |
| | | | | of the actA |
| | | | | promoter |
| | | | | at ActA locus |
| CRS100 | DP-L4056 | none | n/a | deleted ActA, |
| | | | | deleted InlB |
| DP-L4027 | DP-L4056 | none | n/a | deleted hly, |
| | | | | cannot escape |
| | | | | from |
| | | | | phagolysosome |
| DP-L4056 | 10403S | none | n/a | Phage cured |
| BH225 | CRS-100 | pBHE292 | tRNA^{arg} | PSA holin-lysin |
| | | | | under the control |
| | | | | of actA |
| | | | | promoter |
| BH226 | CRS-100 | pBHE292 | tRNA^{arg} | PSA holin-lysin |
| | | | | under the control |
| | | | | of actA |
| | | | | promoter |
| BH276 | DP-L4056 | pBHE292 | tRNA^{arg} | PSA holin-lysin |
| | | | | under the control |
| | | | | of actA |
| | | | | promoter |
| BH279 | ActAN100_OVA | pBHE292 | tRNA^{arg} | PSA holin under |
| | (AH1A5) | | | the control of |
| | | | | actA promoter |
| BH334 | DP-L4056 | pBHE340 | tRNA^{arg} | PSA holin under |
| | | | | the control of |
| | | | | actA promoter |
| BH336 | DP-L4056 | pBHE361 | tRNA^{arg} | PSA lysin under |
| | | | | the control of |
| | | | | actA promoter |
| BH561 | BH225 | pBHE292(cured) | tRNA^{arg} | Erm^{S} derivative |
| | | | | of BH225 |
| BH567 | BH334 | pBHE340(cured) | tRNA^{arg} | Erm^{S} derivative |
| | | | | of BH334 |
| BH721 | DP-L4056 | pBHE631 | comK | contains |
| | | | | luciferase under |
| | | | | the control of the |
| | | | | actA promoter |
| | | | | with an |
| | | | | intervening |
| | | | | IRES |
| BH727 | BH567 | pBHE633 | comK | contains two |
| | | | | copies of holin |
| | | | | under the control |
| | | | | of actA |
| | | | | promoter, one at |
| | | | | tRNA^{arg} and the |
| | | | | other at comK |
| BH741 | BH721 | pBHE631(cured) | comK | Erm^{S} derivative |
| | | | | of BH721 |
| BH743 | BH741 | pBHE633 | comK | contains |
| | | | | luciferase under |
| | | | | the control of the |
| | | | | actA promoter |
| | | | | with an |
| | | | | intervening |
| | | | | IRES at tRNA^{arg}, |
| | | | | holin under |
| | | | | control of actA |
| | | | | promoter at |
| | | | | comK |
| BH745 | BH741 | pBHE636 | comK | contains |
| | | | | luciferase under |
| | | | | the control of the |
| | | | | actA promoter |
| | | | | with an |
| | | | | intervening |
| | | | | IRES at tRNA^{arg}, |
| | | | | holin-lysin |
| | | | | under control of |
| | | | | actA promoter at |
| | | | | comK |
| BH575 | DP-L4056 | pBHE573 | episomal | allows delivery |
| | | | | of luciferase |
| | | | | based eukaryotic |
| | | | | expression |
| | | | | cassette from |
| | | | | WT Listeria to |
| | | | | infected host |
| | | | | cells |
| BH577 | BH276 | pBHE573 | episomal | allows delivery |
| | | | | of luciferase |
| | | | | based eukaryotic |
| | | | | expression |
| | | | | cassette from |
| | | | | holin-lysin |
| | | | | expressing |
| | | | | Listeria to |
| | | | | infected host |
| | | | | cells |
| BH579 | BH334 | pBHE573 | episomal | allows delivery |
| | | | | of luciferase |
| | | | | based eukaryotic |
| | | | | expression |
| | | | | cassette from |
| | | | | holin expressing |
| | | | | Listeria to |
| | | | | infected host |
| | | | | cells |
| BH581 | BH336 | pBHE573 | episomal | allows delivery |
| | | | | of luciferase |
| | | | | based eukaryotic |
| | | | | expression |
| | | | | cassette from |
| | | | | lysin expressing |
| | | | | Listeria to |
| | | | | infected host |
| | | | | cells |

All publications, patents, patent applications, internet sites, and accession numbers/database sequences (including both polynucleotide and polypeptide sequences) cited herein are hereby incorporated by reference herein in their entirety for all purposes to the same extent as if each individual publication, patent, patent application, internet site, or accession number/database sequence were specifically and individually indicated to be so incorporated by reference.

1-112. (canceled)

113. A Listeria bacterium, comprising:
(a) a first polynucleotide comprising (i) a polynucleotide encoding a holin protein, and (ii) a first promoter, wherein the first promoter is operably linked to the polynucleotide encoding the holin protein; and
(b) a second polynucleotide comprising (i) a polynucleotide encoding a heterologous polypeptide, and (ii) a second promoter, wherein the second promoter is operably linked to the polynucleotide encoding the heterologous polypeptide,
wherein the Listeria bacterium displays one or more of the following characteristics:
(i) when the Listeria bacterium expresses the holin protein, the expression of the holin protein does not substantially impair the growth of the bacterium;
(ii) when the Listeria bacterium expresses the holin protein, the cell membrane of the bacterium is not lysed; and
(iii) the holin protein is derived from a non-Listerial bacterium or from a bacteriophage that is not a listeriophage.

114. The Listeria bacterium of claim 113, which further comprises RNA transcripts generated from the second polynucleotide, wherein the RNA transcripts encode the heterologous polypeptide, and wherein, when the holin protein is expressed by the bacterium, at least some of the RNA transcripts are released from the bacterium, wherein the release is holin-dependent.

115. The Listeria bacterium of claim 114, wherein the RNA transcripts comprise an expression cassette derived from an ssRNA positive-strand virus, wherein the expression cassette encodes the heterologous polypeptide.

116. The Listeria bacterium of claim 113, which further comprises the heterologous polypeptide, wherein, when the holin protein is expressed by the bacterium, at least some of the heterologous polypeptide is released from the bacterium, wherein the release is holin-dependent.

117. The Listeria bacterium of claim 116, wherein the heterologous polypeptide does not comprise a signal peptide sequence.

118. The Listeria bacterium of claim 113, wherein the second polynucleotide encodes a self-replicating RNA which encodes said heterologous polypeptide.

119. The Listeria bacterium of claim 113, wherein when the holin protein is expressed by the bacterium, the second polynucleotide is released from the bacterium, wherein the release is holin-dependent.

120. The Listeria bacterium of claim 118, wherein when the bacterium further comprises the self-replicating RNA and the holin protein is expressed by the bacterium, at least some of the self-replicating RNA is released from the bacterium, wherein the release is holin-dependent.

121. The bacterium of claim 113, wherein the Listeria bacterium expresses the holin protein when the bacterium is in the cytosol of an infected host cell.

122. The bacterium of claim 118, wherein the self-replicating RNA comprises an expression cassette derived from an ssRNA positive-strand virus.

123. The bacterium of claim 113, wherein the first polynucleotide is in the genomic DNA of the Listeria bacterium, and wherein the second polynucleotide is in the genomic DNA of the Listeria bacterium or on a plasmid.

124. The bacterium of claim 113, wherein the first promoter is a prfA-dependent promoter.

125. The bacterium of claim 124, wherein the prfA-dependent promoter is an actA promoter.

126. The bacterium of claim 113, wherein the Listeria bacterium is a Listeria monocytogenes bacterium.

127. The bacterium of claim 113, wherein the Listeria bacterium comprises an inactivating mutation in actA, inlB, or both actA and inlB.

128. The bacterium of claim 113, wherein the Listeria bacterium comprises an inactivating mutation in one or more genes selected from the group consisting of uvrA, uvrB, uvrC, and a recombinational repair gene.

129. The bacterium of claim 113, which further comprises: (c) a third polynucleotide comprising (i) a polynucleotide encoding a lysin protein, and (ii) a third promoter, wherein the third promoter is operably linked to the polynucleotide encoding the lysin protein.

130. The bacterium of claim 113, which further comprises a nucleic acid cross-linking agent.

131. A pharmaceutical composition comprising the bacterium of claim 113.

132. A method for inducing an immune response to an antigen in a mammal, comprising administering an effective amount of a composition comprising the bacterium of claim 113 to the mammal.
